(19) United States
(12) Patent Application Publication
(10) Pub. No.: US 2026/0174790 A1
(43) Pub. Date: Jun. 25, 2026

(54) HUMAN AND MURINE OPRM1 PROMOTERS AND USES THEREOF

(71) Applicants: The Trustees of the University of Pennsylvania, Philadelphia, PA (US); The Board of Trustees of the Leland Stanford Junior University, Stanford, CA (US)

(72) Inventors: Gregory Corder, Philadelphia, PA (US); Gregory Salimando, Philadelphia, PA (US); Karl Deisseroth, Stanford, CA (US); Charu Ramakrishnan, San Jose, CA (US)

(21) Appl. No.: 19/128,768

(22) PCT Filed: Nov. 10, 2023
(86) PCT No.: PCT/US2023/079350

§ 371 Date: May 9, 2025

Related U.S. Application Data
(60) Provisional application No. 63/383,462, filed on Nov. 11, 2022.

(51) Int. Cl. A61K 31/7088; A61K 31/5513; A61K 35/76; A61K 48/00; A61P 29/00; C07K 14/015; C07K 14/705; C12N 5/0793; C12N 15/11; C12N 15/864

(52) U.S. Cl. CPC A61K 31/7088; A61K 31/5513; A61K 35/76; A61K 48/0058; A61P 29/00; C07K 14/015; C07K 14/705; C12N 5/0619; C12N 15/11; C12N 15/8645; C12N 2750/14143; C12N 2830/34; C12N 2830/85

(57) ABSTRACT

The disclosure relates to isolated nucleic acids comprising opioid receptor promoter regions and methods and compositions comprising said isolated nucleic acids. In one aspect the invention provides an isolated nucleic acid comprising a first region that encodes a Opioid Receptor promoter (ORP) region operably linked to a second region encoding an exogenous protein; wherein the opioid receptor promoter is selected from the group consisting of a mu opioid receptor promoter (MORP), a delta opioid receptor promoter (DORP), and a kappa opioid receptor promoter (KORP).

### CROSS-REFERENCE TO RELATED APPLICATION

The present application is entitled to priority under 35 U.S.C. § 119(e) to U.S. Provisional Patent Application No. 63/383,462, filed Nov. 11, 2022, which is hereby incorporated by reference in its entirety herein.

### BACKGROUND

Mu-Opioid receptors (MORs) are inhibitory G-protein coupled receptors expressed across molecularly and functionally distinct cell-types in the peripheral (PNS) and central nervous systems (CNS), where they act as the primary effector of endogenous opioid peptides to fine-tune neural transmission, cellular excitability, and influence gene transcription. Exogenous opioid compounds, whether employed clinically or recreationally, leverage this evolutionarily conserved MOR system at non-physiological concentrations and timescales leading to intended benefits like analgesia, and unwanted detriments like reinforcement and respiratory depression. With concurrent global epidemics of chronic pain and opioid use disorders, there is a critical need to disentangle the underlying mechanisms and identify specific cells involved in opioidergic functions, as well as widely adoptable, robust, and reliable methods that can be employed across numerous in vivo and in vitro model systems for neuroscientific investigations. The invention of the present disclosure addresses these needs.

### SUMMARY OF THE INVENTION

As described herein, the present disclosure relates to isolated nucleic acids comprising opioid receptor promoter regions and methods and compositions comprising said isolated nucleic acids.

In one aspect, the present disclosure provides an isolated nucleic acid comprising a first region that encodes a Opioid Receptor promoter (ORP) region operably linked to a second region encoding an exogenous protein, wherein the opioid receptor promoter is selected from the group consisting of a mu opioid receptor promoter (MORP), a delta opioid receptor promoter (DORP), and a kappa opioid receptor promoter (KORP).

In certain embodiments, the exogenous protein is a reporter protein.

In certain embodiments, the reporter protein is selected from the group consisting of GFP, eYFP, mCherry, and GCaMP6f.

In certain embodiments, the first and second regions are separated by a linker sequence.

In certain embodiments, the linker sequence comprises a nucleic acid sequence selected set forth in SEQ ID NOs: 8 and 9.

In certain embodiments, the MORP region is derived from a mouse MORP.

In certain embodiments, the MORP regions is derived from a human MORP.

In certain embodiments, the MORP region comprises a nucleic acid sequence selected from the group consisting of SEQ ID NOs: 6 and 7.

In certain embodiments, the DORP region comprises a nucleic acid sequence selected from the group consisting of SEQ ID NOs: 13 and 14.

In certain embodiments, the KORP region comprises a nucleic acid sequence set forth in SEQ ID NO: 15.

In certain embodiments, the exogenous protein is a designer receptor exclusively activated by a designer drug (DREADD).

In certain embodiments, the DREADD is encoded by a nucleic acid sequence comprising the nucleic acid sequence set forth in SEQ ID NO: 11.

In certain embodiments, the designer drug agonist is selected from the group consisting of clozapine N-oxide (CNO) and deschloroclozapine (DCZ).

In certain embodiments, the reporter protein is encoded by a nucleic acid sequence selected from the group consisting of SEQ ID NOs: 10 and 12.

In certain embodiments, the nucleic acid of the above aspects or embodiments, or any aspect or embodiment disclosed herein further comprises a third region that encodes an additional exogenous protein.

In certain embodiments, the third region is operably linked to the first region.

In certain embodiments, the second region encodes a DREADD, and the third region encodes a reporter protein.

In certain embodiments, the second region encodes a reporter protein, and the third region encodes a DREADD.

In certain embodiments, the second region and third region are separated by a linker sequence.

In certain embodiments, the linker sequence is a nucleic acid sequence selected from the group consisting of SEQ ID NOs: 8 and 9.

In certain embodiments, the isolated nucleic acid comprises a nucleic acid sequence selected from the group consisting of SEQ ID NOs: 3, 4, and 5.

In another aspect, the current disclosure provides a recombinant Adeno-Associated Virus (AAV) vector comprising the isolated nucleic acid of any one of the above aspects or embodiments or any aspect or embodiment disclosed herein.

In certain embodiments, the vector has a tropism for central nervous system (CNS) tissue.

In certain embodiments, the vector comprises an AAV capsid protein of a serotype selected from the group consisting of AAV1, AAV2, AAV4, AAV5, AAV6, AAV7, AAV8, AAV9, AAV9P1, PHP.A, PHP.B, Rh8, Rh10, Rh43, Anc80L65, ShH10, ShH10Y, ShH19, Olig001, DJ, and TM6.

In certain embodiments, the vector comprises an AAV capsid protein of serotype AAV1 or a variant thereof.

In certain embodiments, the vector comprises an AAV capsid protein of serotype AAV5 or a variant thereof.

In another aspect, the current disclosure provides a pharmaceutical composition comprising the recombinant AAV vector of any one of the above aspects or embodiments or any aspect or embodiment disclosed herein and an acceptable carrier or excipient.

In another aspect, the current disclosure provides a method of assessing Opioid Receptor promoter (ORP) activity in a cell, comprising contacting the cell with a vector comprising the isolated nucleic acid of any one of the above aspects or embodiments or any aspect or embodiment disclosed herein, wherein activity of the endogenous opioid receptor promoter drives activity of the ORP region of the isolated nucleic acid and expression of the exogenous protein encoded by the second or third regions, and wherein the opioid receptor promoter is selected from the group consisting of a mu opioid receptor promoter, a delta opioid receptor promoter, and a kappa opioid receptor promoter.

In certain embodiments, the cell is a cell of the central nervous system (CNS).

In certain embodiments, the cell is a neuron.

In another aspect, the current disclosure provides a method of providing analgesia to a subject in need thereof, comprising:
a. administering to the subject an effective amount of a composition comprising a recombinant AAV vector comprising an isolated nucleic acid comprising a first region encoding a ORP regionoperably linked to a second region encoding a designer receptor exclusively activated by a designer drug (DREADD), wherein the DREADD is expressed by cells of the central nervous system; and
b. administering to the subject an effective amount of an agonist capable of engaging and activating the DREADD of part a., thereby providing analgesia, wherein the ORP region is selected from the group consisting of a MORP region, a DORP region, and a KORP region.

In certain embodiments, the composition further comprises a pharmaceutically acceptable carrier or excipient.

In certain embodiments, the AAV vector comprises a capsid protein of a serotype selected from the group consisting of AAV1, AAV2, AAV4, AAV5, AAV6, AAV7, AAV8, AAV9, AAV9P1, PHP.A, PHP.B, Rh8, Rh10, Rh43, Anc80L65, ShH10, ShH10Y, ShH19, Olig001, DJ, and TM6.

In certain embodiments, the AAV vector comprises a capsid protein of serotype AAV1 or a variant thereof.

In certain embodiments, the AAV vector comprises a capsid protein of serotype AAV5 or a variant thereof.

In certain embodiments, the DREADD is DREADD-Gi, also known as hM4Di.

In certain embodiments, the DREADD-Gi is encoded by a nucleic acid sequence comprising the nucleic acid sequence set forth in SEQ ID NO: 11.

In certain embodiments, the agonist is selected from the group consisting of clozapine-N-oxide (CNO) and deschloroclozapine (DCZ).

In certain embodiments, the composition is administered before the administration of the ligand.

In certain embodiments, the subject is a mammal.

In certain embodiments, the subject is human.

### BRIEF DESCRIPTION OF THE DRAWINGS

The following detailed description of selected embodiments of the invention will be better understood when read in conjunction with the appended drawings. For the purpose of illustrating the invention, non-limiting embodiments are shown in the drawings. It should be understood, however, that the invention is not limited to the precise arrangements and instrumentalities of the embodiments shown in the drawings.

FIGS. 1A-1N illustrate the development and validation of murine and human mu opioid receptor promoter (MORp) driven viral constructs. FIG. 1A, DNA sequence for the murine Oprm1 (upper) and human OPRM1 (lower) promoter regions, including the approximate locations of several transcriptional elements including repressor and activator transcription factors (TFs), transcription start sites and promoter elements, as determined via PROMO and Eukaryotic Promoter Database & UCSC Genome Browser analyses of the murine and human genes (FIG. 8). The promoter region encoded by the four murine promoter constructs (mMORp1-4) and single human promoter construct (hMORp) are depicted beneath each sequence map. FIG. 1B, mMORp and hMORp construct designs and packaging schema within adeno-associated viral (AAV) vectors of multiple different capsid serotypes. FIG. 1C, Transduction efficacy from initial in vivo intracranial injections of the mMORp1-4-eYFP constructs into the inbred C57BL/6J mouse medial prefrontal cortex (mPFC), scale bar=500 um. FIG. 1D, mPFC expression pattern with an optimized titer of AAV1-mMORp1¬eYFP across mPFC subregions, including the cingulate (Cg1), prelimbic (PL) and infralimbic (IL) cortex, scale bar=200 um. FIG. 1E, Higher magnification images of the mPFC following transduction with optimized titer of the mMORp1 viral construct. Amplification of the eYFP signal, along with staining for both neuronal (NeuN) and microglial (Iba1) markers demonstrate selective transduction of neurons. Cortical layer division markers (Layers 1-6) highlight viral spread and efficiency, scale bar=100 um. FIG. 1F, Overlap of mMORp1-eYFP viral expression and endogenous mu opioid receptor (MOR) immunoreactivity within the central amygdala (CeA) (denoted by blue outline), but not surrounding amygdalar subregions of a C57BL/6J mouse, using a knock-out mouse-validated anti-MOR antibody (FIG. 10). Basolateral amygdala (BLA), medial anterodorsal amygdala (MeAd), medial anteroventral amygdala (MeAv), basomedial amygdala (BMA), dorsal entopeduncular nucleus (EPd), ventral entopeduncular nucleus (EPv), intercalcated cells (ITCs), globus pallidus externa (GPe), substantia innominota (SI), and lateral hypothalamus (LH); scale bar=200 um. FIG. 1G, RNAscope FISH in the CeA of a mMORp1-eYFP injected mouse examining co-localized of Oprm1 and eYfp mRNA transcripts. FIGS. 1H-1I, CeA co-localization of AAV5-mMORp1-hM4Di-mCherry and Oprm12A-Cre:Sun1-sfGFP reporter nuclei (FIG. 1H) or anti-Cre staining (FIG. 1I). j-m, Co-expression of AAV5-mMORp1-hM4Di-mCherry with anti-Cre immunoreactive cells (Oprm1Cre mice) in the CeA (FIG. 1J), dorsomedial striatum (DMS) (FIG. 1K), mPFC (FIG. 1L) and ventral tegmental area (VTA, FIG. 1M). FIG. 1N, Averaged number of mMORp1-mCherry+/anti-Cre+ cells compared to mMORp1-mCherry+/anti-Cre− cells quantified from successfully transduced brain regions of interest (from left or right hemispheres, or both) of Oprm1Cre and Oprm12A-Cre:Sun1 mice within the CeA (˜90.2%, N=5, n=9), DMS (˜89.65%, N=5, n=7), mPFC (˜88.28%, N=3, n=4), and VTA (˜85.24%, N=5, n=8). Detailed information of total counts and quantification for each region within individual mouse lines can be found in FIG. 1I). Scale bar=100 um for FIGS. 1G-1M.

FIGS. 2A-2E illustrate mMORp viral transduction within putative MOR+ cells in multiple brain regions across mammalian model organisms. FIG. 2A, Mouse (C57BL/6J) CeA expression of AAV1-mMORp-eYFP (titer: 1×1011 gc/mL). Left: overview of the CeA (blue borders) and surrounding regions (white borders). Right: higher magnification images of mMORp-eYFP (anti-GFP amplified) with neuronal (NeuN) and microglial (Iba1) cell-type markers. FIG. 2B, Mouse VTA expression of AAV1-mMORp-eYFP: Left: VTA (magenta borders). Right: anti-GFP amplified eYFP, NeuN and Iba1. FIG. 2C, Rat (Sprague-Dawley) CeA expression of AAV1-mMORp-eYFP (titer: 1×1012 gc/mL). Left: overview of the CeA (blue borders) and surrounding structures. Right: anti-GFP amplified eYFP, NeuN and Iba1. FIG. 2D, Rat VTA expression of mMORp-eYFP. Left: overview of VTA (magenta borders). Right: anti-GFP amplified eYFP, NeuN and Iba1. FIG. 2E, Shrew (Asian house shrew) area postrema/nucleus tractus solitarius (AP/NTS) expression of AAV1-mMORp-eYFP (titer: 1×1012 gc/mL). Left: overview of NTS (purple borders) and surrounding structures. Right: anti-GFP amplified eYFP, NeuN and Iba1. Scale bars=100 um (far left), 200 um (right) for FIGS. 2A-2E.

FIGS. 3A-3G illustrate mMORp-hM4Di spinal cord expression and chemogenetic-induced analgesia. FIG. 3A, AAV1-mMORp-hM4Di-mCherry injection schema within the lumbar spinal cord in C57BL/6J mice to inhibit dorsal horn MOR+ cells and not MOR+ nociceptors or descending brain stem circuits. FIG. 3B, mMORp-hM4Di-mCherry expression and spread across the L4 spinal cord; scale bar=100 um. FIG. 3C, Location map and quantification of mMORp¬-hM4Di-mCherry+ cells across the Rexed laminae in the dorsal and ventral horns (N=3 mice). FIG. 3D, Experimental timeline for viral injections and chemogenetic behavior testing. FIG. 3E, Mechanical sensory thresholds (von Frey Up-Down testing) in mMORp¬-hM4Di-mCherry injected mice (N=9 mice) compared with hSyn-mCherry injected controls (N=7 mice) at baseline and 30 minutes following CNO administration (3 mg/kg; Two-way ANOVA+Bonferroni: main effect: P=0.011 [viral treatment×CNO treatment]; multiple comparisons: basal v. CNO, P=0.990 [mCherry], P=0.006 [hM4Di]). Average response changes per group shown as thick gray (mCherry) or red (hM4Di) lines. Individual mice are shown as thin gray and red lines. FIG. 3F, Nocifensive behaviors on an inescapable 52.5° C. hot plate over a 30-see trial: latency (sec) to hind paw withdrawal (two-way ANOVA+Bonferroni: main effect: P=0.085 [viral treatment], P=0.162 [CNO treatment]; multiple comparisons: basal v. CNO, P>0.999 [mCherry], P=0.067 [hM4Di]), hind paw licking duration (two way ANOVA+Bonferroni; main effects: P=0.008 [viral treatment×CNO treatment], P=0.008 [viral treatment]; multiple comparisons: basal v. CNO, P>0.999 [mCherry], P=0.0007 [hM4Di]), and total jumping bouts (two way ANOVA+Bonferroni; main effects: P=0.004 [viral treatment×CNO treatment], P=0.002 [viral treatment], P=0.006 [CNO treatment]; multiple comparisons: basal v. CNO, P=0.0006 [mCherry], P>0.999 [hM4Di]). FIG. 3G, Time course (left) and cumulative global scoring (right) of nocifensive behaviors (licking, biting, jumping, etc.) observed in the formalin injection test during the first and second phases of testing in mMORp-hM4Di-mCherry and control animals at basal and post-CNO time points (Time course: two-way ANOVA+Bonferroni; main effects: P=0.006 [time bin×nocifensive behaviors], P<0.0001 [time bin], P=0.001 [nocifensive behaviors], multiple comparisons: mCherry v. hM4Di, P=0.014 [5 min], P=0.008 [20 min], P=0.0002 [25 min], P=0.48 [30 min]. Global scoring: two-way ANOVA+Bonferroni; main effects: P=0.006 [stage×nocifensive behaviors], P<0.0001 [stage], P=0.001 [nocifensive behaviors]; multiple comparisons: mCherry v. hM4Di, P=0.222 [1st stage], P<0.0001 [2nd stage]). All data are presented as means s.e.m. *P<0.05, **P<0.01, ***P<0.001, ****P<0.0001.

FIGS. 4A-4G illustrate mMORp-GCaMP6f in vivo photometry recording of MOR+ neural population calcium activity in response to noxious stimulation, opioid administration, and opioid withdrawal. FIG. 4A, In vivo fiber photometry recording set up and overall experimental design, as well as AAV1/5-mMORp-GCaMP6f injection schema in C57BL/6J mice. FIG. 4B, mMORp¬GCaMP6f viral targeting and transduction efficiency within CeA neurons (NeuN+) and fiber optic implant placement; scale bars=500 um (left), 100 um (right). FIG. 4C, Photometry experimental timeline for mMORp-GCaMP6f (N=12) and mMORp-eYFP (N=3) injected mice. FIG. 4D, Normalized calcium-mediated activity responses observed in the CeA of mMORp-GCaMP6f and mMORp-eYFP mice in response to 10 air puff applications (1-min inter-stimulation interval. Thick lines=averaged responses, thin lines=individual mice average responses to all 10 stimulations). Inset graphs depict differences in the area under the curve (AUC) quantified between averaged GCaMP6f and eYFP control response (two tailed unpaired t-test, P=0.035) and peak z-score between groups (two tailed unpaired t-test, P=0.011). FIG. 4E, Plots for normalized, calcium-mediated responses to the application of 10˜55° C. hot water droplets to the left hind paw (1-min inter-stimulation interval). Thick lines=average group response, thin lines=individual mice averaged response. Insets show AUC (two tailed unpaired t-test, P=0.051) and peak z-score (two tailed unpaired t-test, P=0.009) comparisons for eYFP and GCaMP6f expressing mice. FIG. 4F, Normalized, calcium-mediated responses to an additional round of hot water hind paw stimulations in the mMORp¬-GCaMP6f mice (N=12) before and 30 mins after morphine (i.p., 10 mg/kg). Opioid-naïve responses (green) and post-morphine responses (blue) are shown as grouped averages (thick lines) and individual averages (thin lines) following 10 stimulations. Insets show AUC (two tailed unpaired t-test, P=0.016) and peak z-scores (two tailed unpaired t-test, P=0.005) comparisons between the pre- and post-morphine responses in mMORp¬GCaMP6f mice. Gray lines track changes in AUC and peak z-score for individual mice in pre- and post-morphine treatment conditions. FIG. 4G, Normalized, calcium-mediated responses from mMORp-GCaMP6f mice undergoing an escalating forced morphine drinking paradigm (FIG. 4c) for morphine drinking (red, N=6 mice) or saccharin drinking (gray, N=5 mice) subjects following naloxone induced precipitated withdrawal (i.p., 3 mg/kg). Inset shows the total calcium-mediated events post-naloxone between morphine and saccharin mice (two tailed unpaired t-test, P=0.0007). All data are presented as means s.e.m. *P<0.05, **P<0.01, ***P<0.001.

FIGS. 5A-5H illustrate mMORp driven recombinases and PHP.eB/PHP.S capsid packaging support intersectional viral strategies for gaining genetic access to CNS and PNS opioidergic cells/circuits. FIGS. 5A-5B, C57BL/6J mice injected with a mix of AAV8-mMORp-mCherry-IRES-Cre and a Cre-dependent AAV9-hDlx-FLEx-eGFP with intersectional expression in putative MOR+/GABAergic neurons in mPFC (FIG. 5A) and somatosensory cortex (FIGS. 8, 5B). Insets in FIGS. 5A and 5B show higher magnification images of hDlx-FLEx-eGFP cells (green) overlap with mMORp-mCherry-IRES-Cre cells (magenta); scale bars=200 μm and 100 um for insets. FIGS. 5C-5D, Somatostatin-Cre (Sst-Cre) transgenic mice injected in mPFC (FIG. 5C) or S1 (FIG. 5D) with a 9:1 mix of AAV1-mMORpI-FlpO and an INTERSECT Cre- and Flp-dependent AAV8-hSyn-Con/Fon-eYFP. Inset high magnification images show intersectional eYFP+ cells. FIG. 5E, CNS expression in representative sections from the spinal cord (coronal) and brain (sagittal along the medial-lateral axis relative to Bregma) of Oprm1-Cre transgenic mice injected retro-orbitally with a 1:1 mix of AAV.PHP.eB-mMORp-eYFP and AAV.PHP.eB-CAG-FLEx-tdTomato viruses (1:1); scale bars=500 um (spinal cord sections) and 1000 um (sagittal sections). FIGS. 5F-5G, PNS dorsal root ganglia (DRG) FISH from C57BL/6J mice with intracerebroventrical injection of either AAV.PHP.S-mMORp-eYFP (FIG. 5F) or AAV.PHP.S-CAG-tdTomato (FIG. 5G). Custom cDNA probes targeting Rbfox3 (NeuN), Oprm1, eYfp and tdTomato transcripts; scale bars=50 um. FIG. 5H, Quantification of total Oprm1(+) cells co-labeled for tdTomato in control animals (N=2 mice, n=6 ROIs) compared to total Oprm1+ cells co-labeled for eYfp in PHP.S-mMORp-eYFP injected mice (N=2 mice, n=4 ROIs) across treatment groups (two tailed unpaired t-test, P=0.0011). Data presented as means±s.e.m., **P<0.01.

FIGS. 6A-6G illustrate hMORp viral constructs drive robust transduction of putative opioidergic cells in non-human primate neural tissue. FIG. 6A, 3D reconstruction of the skin, skull and underlying vasculature of a rhesus macaque for pre-operative intracranial injection planning. FIG. 6B, Post-injection manganese-enhanced MRI for AAV1-hMORp-eYFP in vivo targeting accuracy assessment (virus mixed 1:100 with 100 mM manganese solution). FIG. 6C, Left: Dorsal anterior cingulate cortex (dACC) expression of hMORp-eYFP; scale bar=1000 um. Right: dACC, higher magnification with cortical layer markers; scale bar=200 um. FIG. 6D, Co-expression of hMORp-eYFP with neuronal (NeuN) but not microglial (Iba1) markers; scale bar=100 um. FIG. 6E, RNAscope FISH in dACC tissue for co-expression of YFP, OPRM1, and SLC17A7 (VGLUT1) mRNA transcripts; scale bar=100 um, far right image=digital zoom of merged image. FIG. 6F, Summary quantification of the total EYFP transcript positive cells quantified in sample regions of interest within dACC (upper, N=1 dACC slice, n=4 ROIs) with either EYFP+/OPRM1− (18.6%) or EYFP+/OPRM1+(81.4%) to demonstrate hMORp-eYFP virus expression specificity. FIG. 6G, hMORp-eYFP expression within the basal nucleus of the amygdala, the insular cortex and the mediodorsal thalamus following intracranial viral injections into these putative MOR expressing regions. Scale bar=1000 um.

FIGS. 7A-7H illustrate mMORp viral transduction in human iPSC-derived MOR+ nociceptors. a-b, Representative low (left, scale bar=50 um) and high (right, scale bar=100 um) magnification images of cultured human nociceptors treated with higher titer (1×1012 gc/mL) AAV.PHP.S-mMORp¬-eYFP virus (FIG. 7A), or an AAV.PHP.S-CAG-tdTomato virus (FIG. 7B). High magnification sample regions are denoted in lower magnification images via a white box. c-d, Similar low (left) and high (right) magnification images of cultured nociceptors treated with lower titer mMORp-eYFP (1×109 gc/mL, FIG. 7C) or CAG-tdTomato virus (FIG. 7D). FIGS. 7E-7F, Images of cultured human cardiomyocytes treated with high titer mMORp-eYFP (FIG. 7E) or CAG-tdTomato (FIG. 7F) viruses, with regions boxed in white denoting high magnification sample areas shown on the right. g-h, Images of cardiomyocytes treated with low titer mMORp-eYFP (FIG. 7G) or CAG-tdTomato (FIG. 7H) viruses, at both low (left) and high (right) magnification.

FIGS. 8A-8D illustrate Analysis of Oprm1 genetic sequence with PROMO and EPD for identifying target regions for mMORp1-4 construct designs. FIG. 8A, Readout run on the DNA sequence of the mouse Oprm1 gene in order to identify putative transcription factor binding sites (TFBS) using the PROMO database17. Identified transcription factors are numbered and listed in a color-coded manner, while their putative location(s) within the Oprm1 genetic sequence are tabulated below, including their position along the gene. FIG. 8B, Screen capture of readout produced by the Eukaryotic Promoter Database18 following a query for information on the promoter sequence of the Oprm1 gene found in mice (Mus musculus). Pertinent information on the complete promoter sequence is provided and was used to guide the design of our mMORp1-4 and hMORp1 constructs to make them as complementary to desirable locations within the Oprm1 and OPRM1 promoter sequences, while avoiding regions containing potential splice sites that could hamper construct specificity. FIGS. 8C-8D, Linear plasmid maps for the mMORp1-eYFP (FIG. 8C) and hMORp1-eYFP (FIG. 8D) constructs in an AAV backbone.

FIGS. 9A-9K illustrate mMORp1 and hMORp1 viral constructs show robust expression in neuronal cell cultures in vitro and additional brain regions of interest in vivo. a-b, Transduced cultured primary cortical cell cultures from P0 rat pups with AAV1-mMORp1-eYFP (FIG. 9A) or AAV1-hMORp-eYFP (FIG. 9B). Staining for nuclei (DAPI, gray) and basal eYFP expression across cultures demonstrate the ability of both viruses to transduce neurons within culture; scale bars=50 um. FIGS. 9C-9H, Additional representative images from high titer/volume injections of AAV1-mMORp1-eYFP (FIGS. 9C-9E) or AAV1-hMORp1-eYFP (FIGS. 9F-9H) into putative mu opioid receptor (MOR) positive regions in vivo in C57BL/6J mice, including medial prefrontal cortex (mPFC FIGS. 9C, 9F), central amygdala (CeA, FIGS. 9D, 9G), and ventrolateral periaqueductal gray (vlPAG, e,h), with basal reporter signal shown in green, and DAPI nuclei staining in blue. Restriction/overall spread of the virus within targeted regions is shown via overlaid anatomical borders on higher magnification images. L1-L6=cortical layers, Cg1=cingulate area 1, Cg2=cingulate area 2, CeC=central capsular division of the CeA, CeL=central lateral division of the CeA, CeM=central medial division of the CeA, BLA=basolateral amygdala, LA=lateral amygdala, EPd=dorsal enteropeduncular nucleus, dPAG=dorsal periaqueductal gray, 1PAG=later periaqueductal gray, DR=dorsal raphe nucleus; scale bars=500 um (left), 200 um (right) for c-e, 200 um (left) and 100 um (right) for FIGS. 9F-9H, 9I-9K, Representative images of AAV1-mMORp1-eYFP transduced cells in mouse mPFC stained for NeuN and Iba1 at lower (left, scale bar=100 um) and higher (right, scale bar=100 um) magnification (FIG. 9I), demonstrating the overlap of mMORp1-eYFP signal in cells stained positively for NeuN (FIG. 9J, white arrows), but not in Iba1 positive cells (FIG. 9K, yellow arrows). Orange arrows indicate mMORp1¬-eYFP positive cell bodies. Digitally magnified images on the far right show boxed regions in FIG. 9K of Iba1+ cell bodies adjacent to, but not overlapping with, mMORp1-eYFP signal in virally transduced cells.

FIGS. 10A-10D illustrate that the Mu opioid receptor (MOR) signal is readily detectable in MOR wildtype mice but absent in Oprm1Cre/Cre homozygous total knockouts. FIGS. 10A-10B, Representative tile scan images of tissue from C57BL/6J mice containing the dorsomedial striatum (FIG. 10A) or the medial thalamus and amygdala (FIG. 10B). Anti-MOR (Abcam, ab134054) and DAPI staining are shown as individual channel and merged to highlight the ability of this antibody to detect MOR within/around the striosomal structures in the striatum, the periventricular nucleus of the thalamus, and the central nucleus of the amygdala, regions known to harbor moderate to high levels of MOR expression. FIGS. 10C-10D, Similar representative images from an Oprm1Cre animal homozygous for the Cre allele (Oprm1Cre/Cre), which is known to produce a complete MOR knockout in this line. MOR detection is completely lost in the same brain regions within this mouse when using the same antibody stain. Scale bars=500 um.

FIGS. 11A-11D illustrate that the Oprm12A-Cre:TRAP-GFP and Oprm12A-Cre:Sun1 mouse lines demonstrate transgene expression in putative Oprm1 and MOR positive cell populations from fluorescent in situ hybridization and immunohistochemistry studies. FIGS. 11A-11B, Representative low (left) and high (right) magnification images from RNAscope fluorescent in situ hybridization studies performed on tissue from the habenula (FIG. 11A) and medial thalamus (FIG. 11B) of Oprm12A-Cre:TRAP-GFP mice following activation of the Translating Ribosome Affinity Purification (TRAP) construct with tamoxifen to drive GFP expression90. Both individual channels for Oprm1 and Gfp transcript staining, as well as a merged image to show of cells (denoted by DAPI nuclear staining in gray) co-labeled for both transcripts are shown in habenula and thalamus containing tissue samples. Low magnification images with anatomical border overlays demonstrate the restriction of Gfp transcript staining to putative Oprm1 transcript positive areas within each region. Labeled structures in each image include, (FIG. 11A): Hbx=subdivisions of the habenula, MHb=medial habenula, LHb=lateral habenula, (FIG. 11B): PVT=paraventricular thalamus, cMD=central nucleus of the mediodorsal thalamus, mMD=medial nucleus of the mediodorsal thalamus, IMD=lateral nucelus of the mediodorsal thalamus. Scale bars=200 um (left), 100 um (right) FIGS. 11C-11D, Representative images for anti-MOR staining conducted on slices taken from the mediodorsal striatum (DMS, FIG. 11C) and parabrachial nucleus (PBN, FIG. 11D) of Oprm12A-Cre:Sun1 reporter mice, which express a nuclear targeted GFP tag in cells expressing the Oprm1 promoter. Low magnification images (left) show the location of higher magnification (right) image boxed in yellow, with individual and combined channels displaying anti-MOR and Sun1-GFP reporter signal separately and combined to demonstrate the expression of the reporter within putative MOR positive brain regions, as well as in substructures within each of these regions known to highly express MOR (i.e. striosomes seen in FIG. 11C). Scale bars=500 um (left), 100 um (right).

FIGS. 12A-12h illustrate the quantification of mMORp-mCherry+ cell populations observed in the CeA, DMS, mPFC and VTA of Oprm12A-Cre and Oprm1Cre mice. FIGS. 12A-12D, Average counts of total mMORp-mCherry positive cell populations quantified across all four regions of interest from viral specificity validation studies performed in Oprm12A-Cre:Sun1 reporter mice injected with AAV1-mMORp-hM4Di-mCherry, with percentile averages shown in parts of a whole charts on the left, and averages for individual cell population counts tabulated in bar graphs on the right. Population counts were performed by counting all cells positive in designated ROIs for mMORp-mCherry expression, as well as all those positive for mCherry and anti-Cre staining or Sun1-GFP reporter alone or together, with results for the CeA (˜92.8%, N=2 mice, n=4 ROIs, FIG. 12A), DMS (˜87.3%, N=2, n=4, FIG. 12B), mPFC (˜97.6%, N=1, n=1, FIG. 12C) and VTA (˜89.7%, N=2, n=3, FIG. 12D) displayed. FIGS. 12E-12H, Similar graphs and charts of averaged population counts and percentiles for mMORp-mCherry+ cells in the CeA (˜86.4%, N=3, n=5, FIG. 12E), DMS (˜90.4%, N=3, n=3, FIG. 12F), mPFC (˜86.8%, N=2, n=3, FIG. 12G) and VTA (˜81.7%, N=3, n=5, FIG. 12H) of Oprm1Cre mice co-injected with a mix of AAV1-mMORp-hM4Di-mCherry and AAV5-hSyn-DIO-eGFP viruses in each region of interest. Population counts were performed by counting all cells positive in designated ROIs for mMORp-mCherry and eGFP expression, as well as all those positive for mCherry or eGFP and anti-Cre staining or Sun1-eGFP reporter alone or together. mMORp¬mCherry+/anti-Cre+ cell populations from both lines were combined to perform the analyses discussed and presented in FIG. 1N.

FIGS. 13A-13B illustrate that the activation of mMORp-hM4Di in spinal cord neurons via intrathecal deschloroclozapine (DCZ) produces analgesic effects similar to CNO treatment. FIG. 13A, Mechanical sensory thresholds (von Frey Up-Down testing) in mMORp-hM4Di-mCherry injected mice (N=9 mice) compared with hSyn-mCherry injected controls (N=7 mice) at baseline and 30 minutes following DCZ administration (10 pg; Two-way ANOVA+Bonferroni: main effect: P=0.033 [viral treatment×DCZ treatment], P=0.011 [DCZ treatment], P=0.030 [viral treatment]; multiple comparisons: basal v. DCZ, P>0.999 [basal], P=0.004 [DCZ]). Average response changes per group shown as thick gray (mCherry) or blue (hM4Di) lines. Individual mice are shown as thin gray and blue lines. FIG. 13B, Nocifensive behaviors on inescapable 52.5° C. hot plate over a 30-sec trial: latency (sec) to hind paw withdrawal (two-way ANOVA+Bonferroni: main effect: P=0.0003 [viral treatment×DCZ treatment], P=0.0007 [DCZ treatment]; multiple comparisons: basal v. DCZ, P>0.999 [basal], P=0.0005 [DCZ]), hind paw licking duration (two way ANOVA+Bonferroni; main effects: P=0.022 [viral treatment×DCZ treatment]; multiple comparisons: basal v. DCZ, P=0.693 [basal], P=0.170 [DCZ]), and total jumping bouts (two way ANOVA+Bonferroni; main effects: P=0.045 [DCZ treatment]; multiple comparisons: basal v. DCZ, P>0.999 [basal], P=0.067 [DCZ]). All data are presented as means±s.e.m., *P<0.05.

FIGS. 14A-14B illustrate that macaque intracranial injection targeting vectors and post-operative imaging. a-b, 3D reconstructions of the macaque skin (tan), skull (white) and vasculature (red) from T1 MRI along injection trajectories calculated to deliver both mMORp and hMORp-eYFP viruses to the anterior cingulate cortex (blue), insular cortex (purple), mediodorsal thalamus (pink) and amygdala (orange) across the first (top, FIG. 14A) and second (bottom, FIG. 14B) surgical procedures performed on the subject (left). Images from post-operative manganese-enhanced MRI scans performed to validate injection accuracy are shown on the right for each targeted region across three planes of view. Manganese contrast signal (shown in light blue, 100 mM solution mixed 1:100 with each virus) serves as an in vivo marker of where each virus was delivered relative to the planned injection sites (colored dots).

FIGS. 15A-15B illustrate the visualization of hMORp-eYFP viral expression within the macaque anterior cingulate cortex. FIG. 15A, Additional low (left) and high (right) magnification images of macaque dorsal anterior cingulate cortex (dACC) tissue following injection of the hMORp-eYFP virus. Expression of eYFP reporter tagged cell bodies (antibody staining enhanced, white) can be noted across cortical layers (blue boxed region), and processes extending up from cortical layer VI neurons into the upper cortical layers can also be observed (yellow boxed regions). Scale bar=200 um (left), right=digital zoom on boxed regions. FIG. 15B, Low and high magnification imaging of transduced cell bodies within layer V of the dACC. Scale bars=1000 um (left), 200 um (middle), 100 um (yellow box), 100 um (blue box).

FIGS. 16A-16B illustrate the visualization of hMORp-eYFP viral expression within the macaque insular cortex and amygdala. FIG. 16A, Additional low and high magnification images of macaque insular cortex tissue following injection of the hMORp-eYFP virus. Processes and cell bodies of eYFP transduced neurons (antibody staining enhanced, white) within layer Vb are shown at both lower magnification (left, scale bars=1000 um [upper], 200 um [lower]) and higher magnification (right, scale bars=100 um [dashed boxes], 100 um [pink box]). FIG. 16B, Additional images of macaque amygdala following hMORp-eYFP transduction. Visualization of transduced cell bodies and processes (white) can be noted at lower magnification (left, scale bars=1000 um [upper], 200 um [lower]), and at higher magnification (right), including detailed imaging of these individual bodies and processes (scale bars=100 um [dashed box, lower left], 100 um [dashed box, upper right]).

FIGS. 17A-17B illustrate the quantification of hMORp-eYFP transduced OPRM1+ cells within macaque anterior cingulate cortex. FIG. 17A, Lower magnification images (upper left) of macaque tissue containing the dorsal anterior cingulate cortex (dACC) processed for RNAscope FISH in order to visualize cells positive for OPRM1, EFYP or VGLUT1 mRNA transcript expression. Cortical layer borders are shown to demonstrate the spread of positively transduced neurons within the macaque dACC following a single injection of the hMORp-eYFP virus (upper right). Graphs tabulating the average and individual total cells labeled for OPRM1 and EYFP transcript counted within four representative regions of interest (ROI1-4) are shown on the right. Averaged ROI counts represent the combined scoring from four independent experimenters analyzing each ROI for the single or co-labeled groups of interest designated in the bar graph. Individual ROI counts show the total counts for each of these groups made by the experimenters across each ROI separately. Scale bar=100 um. FIG. 17B, Merge and single channel higher magnification images from ROIs 1-4 used to produce the counts tabulated above. All cell bodies denoted by DAPI staining were counted and assessed for both labeling and co-labeling for all transcripts of interest within the boundaries of each 20×ROI image. Scale bar=100 um. All data in bar graphs are presented as means s.e.m.

FIG. 18 is a fluorescence micrograph showing the expression of AAV5-KORp-eYFP. Confocal 63× image of the claustrum. Grey=endogenous Oprk1 mRNA, Light grey=Yfp mRNA (from the KORp-YFP), Dark grey=dapi.

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, illustrative materials and methods are described herein. In describing and claiming the present invention, the following terminology will be used.

It is also to be understood that the terminology used herein is for the purpose of describing particular embodiments only and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “AAV vector” as used herein refers to a polynucleotide vector comprising one or more genes of interest (or transgenes) that are flanked by AAV terminal repeat sequences (ITRs). AAV vectors can be produced and packaged into infectious viral particles when present in a host cell that has been transfected with one or more helper plasmids encoding and expressing rep and cap proteins and one or more proteins from adenovirus open reading frame E4 open reading frame 6. The AAV vectors may be operably linked to promoter and enhancer sequences that can regulate the expression of the protein encoded by the AAV vector.

The terms “AAV virion” or “AAV viral particle” or “AAV vector particle” as used herein refers to a viral particle composed of capsid proteins from at least one AAV serotype surrounding a polynucleotide AAV vector. If the particle comprises a heterologous polynucleotide (i.e., a polynucleotide other than a wild-type AAV genome such as a transgene to be delivered to a mammalian cell), it is typically referred to as an “AAV vector particle” or simply an “AAV vector.” Thus, production of AAV vector particle necessarily includes production of AAV vector, as such a vector is contained within an AAV vector particle.

The term “packaging” as used herein refers to intracellular process by which viral virions or particles (e.g., AAV virions or particles), especially viral vector particles or virions are assembled in a host cell. “Packaging” cells comprise the polynucleotide (e.g., helper plasmids) and protein components necessary to assemble functional viral virions.

By “agent” is meant any nucleic acid molecule, small molecule chemical compound, antibody, or polypeptide, or fragments thereof.

By “alteration” or “change” is meant an increase or decrease. An alteration may be by as little as 1%, 2%, 3%, 4%, 5%, 10%, 20%, 30%, or by 40%, 50%, 60%, or even by as much as 70%, 75%, 80%, 90%, or 100%.

As used herein, the term “autologous” is meant to refer to any material derived from the same individual to which it is later to be re-introduced into the individual.

“Allogeneic” refers to a graft derived from a different animal of the same species.

“Xenogeneic” refers to a graft derived from an animal of a different species.

By “biologic sample” is meant any tissue, cell, fluid, or other material derived from an organism.

As used herein, the term “cassette” or “expression cassette” or “regulatory cassette” refer to distinct nucleic acid vectors consisting of a payload transgene and regulatory sequences (i.e., promoter, enhancers, terminators, and the like) which control its expression. Upon successful insertion into a host cell, the regulatory sequences allow for the transcription and translation of the payload transgene.

As used herein, the term “conservative sequence modifications” is intended to refer to nucleic acid or amino acid modifications that do not significantly affect or alter the functional characteristics of the nucleic acid sequence or protein comprising the amino acid sequence. Such conservative modifications include amino acid or nucleic acid substitutions, additions and deletions. Modifications can be introduced into a nucleic acid sequence or protein encoded thereof of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine).

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to, anti-tumor activity as determined by any means suitable in the art.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

By the term “modified” as used herein, is meant a changed state or structure of a molecule or cell of the invention. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

The term “epigenetic” as used herein refers to heritable influences on gene expression that do not involve alterations in DNA nucleotide sequence. Epigenetic regulation can enhance or inhibit expression of affected genes, and can involve chemical modifications of the deoxyribose backbone of the DNA or the association of DNA/histone protein complexes or both.

A “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the plasma membrane of a cell.

By the term “specifically binds,” as used herein with respect to a ligand, is meant a ligand which recognizes a specific receptor or target protein, but does not substantially recognize or bind other molecules in a sample. In some instances, the terms “specific binding” or “specifically binding,” can be used in reference to the interaction of a ligand, a protein, or a peptide with a second chemical species, to mean that the interaction is dependent upon the presence of a particular structure (e.g., a binding domain) on the chemical species; for example, a ligand recognizes and binds to a specific protein structure of a receptor or target protein rather than to proteins generally.

The term “subject” is intended to include living organisms in which an immune response can be elicited (e.g., mammals). A “subject” or “patient,” as used therein, may be a human or non-human mammal. Non-human mammals include, for example, livestock and pets, such as non-human primate, ovine, bovine, porcine, canine, feline and murine mammals. Preferably, the subject is human.

A “target site” or “target sequence” refers to a genomic nucleic acid sequence that defines a portion of a nucleic acid to which a binding molecule may specifically bind under conditions sufficient for binding to occur.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “transgene” refers to the genetic material that has been or is about to be artificially inserted into the genome of an animal, particularly a mammal and more particularly a mammalian cell of a living animal.

The term “transgenic animal” refers to a non-human animal, usually a mammal, having a non-endogenous (i.e., heterologous) nucleic acid sequence present as an extrachromosomal element in a portion of its cells or stably integrated into its germ line DNA (i.e., in the genomic sequence of most or all of its cells), for example a transgenic mouse. A heterologous nucleic acid is introduced into the germ line of such transgenic animals by genetic manipulation of, for example, embryos or embryonic stem cells of the host animal.

The term “knockout mouse” refers to a mouse that has had an existing gene inactivated (i.e., “knocked out”). In some embodiments, the gene is inactivated by homologous recombination. In some embodiments, the gene is inactivated by replacement or disruption with an artificial nucleic acid sequence.

To “treat” a disease as the term is used herein, means to reduce the frequency or severity of at least one sign or symptom of a disease or disorder experienced by a subject.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

In one aspect, the present invention provides isolated nucleic acids comprising promoters derived from mammalian opioid receptor promoter regions (e.g. mu, delta, and kappa opioid receptors) operably linked to one or more transgenes encoding protein products such as reporters, novel receptors, signaling molecules, and the like. In certain embodiments, these nucleic acid constructs can be expressed in cells which normally express mu, delta, and/or kappa opioid receptors, such as various cells of the brain and central nervous system, where they can act as reporters of mu, delta, and/or kappa opioid receptor expression and function. In certain embodiments, the opioid receptor promoter sequences of these nucleic acids can be operably linked to genes encoding designer receptors exclusively activated by a designer drug (DREADDS) in order to selectively activate or block nociceptor activity in a manner more selective manner than traditional opioid or opioid-derivative compounds. In this way, analgesia can be delivered avoiding off-target toxicities and dependency-forming side effects. Also provided are viral vectors (e.g. AAV vectors) capable of delivering the nucleic acid constructs to CNS and brain tissues both in vivo and in vitro.

### Opioid Receptors

Opioid receptors are a family of inhibitory G-protein coupled receptors expressed across several molecularly and functionally distinct cell-types in the peripheral (PNS) and central nervous systems (CNS), where they act as the primary effector of endogenous opioid peptides to fine-tune neural transmission, cellular excitability, and influence gene transcription. The opioid receptor family is considered to comprise three classical receptors (mu, kappa, and delta) and one non-classical receptor (the nociceptin receptor). The mu opioid receptor, also referred to as p or MOP, is encoded by the gene Oprm1 and is named after morphine, it's most commonly recognized exogenous ligand. The delta opioid receptor, also referred to as 6 or DOP, is encoded by the gene Oprd1 and is named after the vas deference, the tissue from which it was first isolated. The kappa opioid receptor, also referred to as κ or KOP, is encoded by the gene Oprk1 and is named after ketocyclazocine, the first identified ligand of this receptor. The nociception receptor, also known as NOP, the nociceptin/orphanin FQ (N/OFQ) receptor, and kappa-type 3 opioid receptor, is encoded by the Oprl1 gene and is the most recently discovered member of the family. Although NOP shares high sequence identity (˜60%) with the three classical opioid receptors, it possesses little or no affinity for opioid peptides or morphine-like compounds such as naloxone. Likewise, classical opioid receptors possess little affinity towards NOP's endogenous ligand nociception.

Exogenous opioid compounds, including the traditional opiate or opioid class drugs, whether employed clinically or recreationally, leverage this evolutionarily conserved DOR/KOR/MOR system at non-physiological concentrations and timescales leading to intended benefits like analgesia, and unwanted off-target like addiction and respiratory depression.

Tools to study the function and biology of opioid receptors (e.g. DOR, MOR, KOR, and NOR) initially relied on pharmacological and radiolabeled reagents to visualize and characterize physiological function in diverse species, such as mice, rats, pigeons, and non-human primates. While recent improvements in the development of transgenic animals, including conditional Oprm1 knock-out, fluorescently tagged MORs and Cre-recombinase lines, continue to refine understanding of opioid receptor neurobiology, most of these methods ultimately rely on behavioral changes to subject animals which are often difficult to measure and interpret. Thus, in some embodiments, the invention of the current disclosure provides polynucleotide constructs, which operably link the promoter regions of the Oprm1 MOR gene, Oprd1 DOR gene, Oprk1 KOR gene, or Oprl1 NOR gene from mouse or human genomes with genes encoding various reporter proteins such as eYFP, mCherry and the like in order to easily visualize and quantify number of MOR+ neurons in a given region of the CNS or PNS. In certain embodiments, these MOR, DOR, and/or KOR promoter regions have been specifically selected or modified in order to be small enough for efficient packaging into AAV vectors, which have a genomic size limit of around 4.7 Kb, while maximizing available genomic space for the other regions encoding exogenous payload proteins. In certain embodiments, the MOR, DOR, and/or KOR promoter regions are used to drive the expression of exogenous receptor molecules, including DREADDs, which allow for the precise expression of engineered receptors in MOR-, DOR-, and/or KOR-expressing cells. The endogenous down-stream signaling pathways which would otherwise be activated via the normal MOR, DOR, and/or KOR/opioid ligand pathway can then be activated by novel, designer ligands of the exogenous receptors. In this way, nociceptor function can be delivered using non-opioid molecules, enabling, for example, analgesia without undesired off-target effects normally associated with traditional opiate-class drugs.

### Designer Receptors Exclusively Activated by Designer Drugs (DREADDs)

In certain aspects, the invention of the current disclosure provides nucleic acid constructs comprising MOR, DOR, or KOR promoter regions operably linked to designer receptors exclusively activated by designer drugs (DREADDs) or receptors activated solely by a synthetic ligand (RASSL). DREADDs are recombinant, exogenous receptor protein molecules that are activated solely by engineered ligand molecules which normally have little to no other biological activity. In this way, the ligand interacts with only the cells which express the engineered receptor protein. In certain embodiments of the present invention, genes encoding the engineered recombinant receptors are under transcriptional control of the MORp, DORp, and/or KORp sequences, which restrict their expression only to cells which endogenously express mu, delta, and/or kappa opioid receptors. DREADD and RASSL systems are typically based on G protein-coupled receptors which have been modified to respond only to synthetic ligands. One non-limiting example of such system is the DREADD-Gi or Gi-DREADD, also known as hM4Di, which is an inhibitory Gi protein coupled receptor derived from the M₄ muscarinic receptor, and is specific for the ligand clozapine N-oxide (CNO) or deschloroclozapine (DCZ). Other non-limiting examples include the kappa-opioid receptor based KOR-DREADD which is selectively activated by the ligand salvinorin B, or the DOR-DREADD. In certain embodiments, the DREADD of the current invention is hM4Di activated by CNO or DCZ. The skilled artisan would be able to select a DREADD or RASSL/ligand system appropriate for use with the invention.

### AAV Vectors

AAV are relatively small, non-enveloped viruses with a ˜4 kb genome that is flanked by inverted terminal repeats (ITRs). The genome contains two open reading frames, one of which provides proteins necessary for replication and the other provides components required for construction of the viral capsid. Wild-type AAV is typically found in the presence of adenovirus as the adenoviruses provide helper proteins that are essential for packaging of the AAV genome into virions. Therefore, AAV production piggy-backs on co-infection with adenovirus and relies on three key elements: the ITR-flanked genome, the open-reading frames, and adeno-helper genes. Due to their non-pathogenic ability to readily infect human cells, AAV is well-studied as a vector for gene delivery. AAV may be readily obtained and their use as vectors for gene delivery has been described in, for example, Muzyczka, 1992; U.S. Pat. No. 4,797,368, and PCT Publication WO 91/18088. Construction of AAV vectors is described in a number of publications, including Lebkowski et al., 1988; Tratschin et al., 1985; Hermonat and Muzyczka, 1984.

AAV-based vector systems typically separate the viral AAV genes, Adenovirus-derived helper genes, and the transgene payload onto two or three separate plasmids. Three plasmid systems consist of an AAV helper plasmid comprising the rep (replication) and cap (capsid) genes, an adenoviral helper plasmid comprising at least the E2a gene, E4 gene, and VA (viral associated) RNA, and a payload plasmid comprising the transgene and associated promoters and enhancers flanked by ITR sequences. The helper plasmid or plasmids do not comprise ITRs in order to prevent packaging of a functional, infectious viral genome.

Two plasmid systems combine the AAV rep and cap genes and adenoviral helper genes onto a single plasmid and simplify viral vector production by reducing the number of transfected plasmids. Often, a dedicated packaging cell line is used which is engineered to express AAV/helper genes prior to introduction of the payload plasmid.

Successful gene therapies require efficient infection of target tissues and establishment of long-term gene expression, and AAV vectors can successfully infect and transduce a broad variety of cell and tissue types, such as brain, liver, and muscle, among others, and have the ability to infect both dividing and quiescent cells. Additionally, AAV-mediated transduction of tissues has been demonstrated to result in long term transgene expression greater than 1.5 years in animal models including canine, murine and hamster.

The tissue tropism of AAV vector particles is influenced by the serotype of the capsid protein, though the receptors and co-receptors that the capsid proteins bind to are often poorly understood and can be expressed by multiple tissue types. For example, AAV2, one of the most well-studied serotypes, has a binding affinity largely for heparan sulfate proteoglycan (HSPG) and as such has a tropism in humans for eye, brain, lung, liver, muscle, and joint tissues. Likewise, AAVs 1, 4, 5, and 6 have a binding affinity largely for sialic acid and a tropism for neuronal tissues while AAVs 5 and 8 share a tropism for skeletal muscle. In this way, the serotype of the AAV capsid protein can be selected to target the payload nucleic acid (e.g., a regulatory cassette) of the AAV vector to a specific tissue or cell type. Alteration or modification of capsid protein structure can also alter the tissue or cellular tropism and affinity of the resulting AAV vector particles.

In certain embodiments, the current disclosure provides AAV vectors comprising a capsid protein derived from AAV1 and variants thereof. AAV1 and capsid proteins based on AAV1 transduce neurons and astrocytes at high efficiency with some studies reporting 75% or more efficiency.

In certain embodiments, the current disclosure provides AAV vectors comprising a capsid protein derived from AAV5 and variants thereof. AAV5 and capsid proteins based on AAV5 make attractive candidates for use as vectors because of their relatively low level of existing neutralizing antibodies in humans and their broad tissue and cell tropism including CNS tissues.

It is contemplated that the nucleic acid-expressing AAV vectors and compositions of the disclosure can be used with any naturally occurring, modified, hybrid, or engineered AAV capsid protein that provides the desired tissue tropism (e.g., neuronal cells, astrocytes, and other cells of the brain and CNS) including, but not limited to AAV1, AAV2, AAV3, AAV3B, AAV4, AAV5, AAV6, AAV7, AAV8, AAV9, AAV9P1, AAV12, AAV-B1, AAV-DJ, AAV-Retro, AAVrh8, AAVrh10, AAVrh25, Anc80L65, LK03, AAVrh18, AAVrh74, AAVrh32.33, AAVrh39, AAVrh43, Oligo001, PHP-B, Spark100, PHP.A, PHP.B, Rh8, Rh10, Rh43, Anc80L65, ShH10, ShH10Y, ShH19, Olig001, DJ, and TM6 among others. The skilled artisan would be able to select an appropriate capsid protein for use with the disclosure based on the desired target tissue or cell type.

### Nucleic Acids and Vectors

The present disclosure provides an isolated nucleic acid comprising a first region encoding a mu opioid receptor promoter region (MORP) operably linked to a second region and, in some embodiments, a third region encoding an exogenous protein such as a reporter protein and/or a DREADD. In certain embodiments, an AAV vector comprises the nucleic acid of the invention. In certain embodiments, the mu opioid receptor promoter region is derived from a mouse. In certain embodiments, the mu opioid receptor promoter is derived from a human. In certain embodiments, the nucleic acid comprises a polynucleotide sequence having at least 80%, 85%, 90%, 95%, 96%, 97%, 98%, or 99% identity to SEQ ID NO: 3, SEQ ID NO: 4, or SEQ ID NO: 5. In certain embodiments, the nucleic acid comprises a polynucleotide sequence set forth in SEQ ID NO: 3, SEQ ID NO: 4, or SEQ ID NO: 5.

In certain embodiments, the invention also provides an AAV vector comprising a nucleic acid encoding a first region encoding a mu opioid receptor promoter region capable of being activated when expressed in cells possessing endogenous mu opioid receptor expression. In certain embodiments, the nucleic acid encoding the MORP is derived from Mus musculus (mMORP), and comprises a polynucleotide sequence having at least 80%, 85%, 90%, 95%, 96%, 97%, 98%, or 99% identity to SEQ ID NO: 6. In certain embodiments, the nucleic acid encoding the MORP comprises the polynucleotide sequence set forth in SEQ ID NO: 6.

In certain embodiments, the invention also provides an AAV vector comprising a nucleic acid encoding a first region encoding a mu opioid receptor promoter region capable of being activated when expressed in cells possessing endogenous mu opioid receptor expression. In certain embodiments, the nucleic acid encoding the MORP is derived from Homo sapiens (hMORP), and comprises a polynucleotide sequence having at least 80%, 85%, 90%, 95%, 96%, 97%, 98%, or 99% identity to SEQ ID NO: 7. In certain embodiments, the nucleic acid encoding the MORP comprises the polynucleotide sequence set forth in SEQ ID NO: 7.

In certain embodiments, the invention also provides an AAV vector comprising a nucleic acid encoding a first region encoding a delta opioid receptor promoter region capable of being activated when expressed in cells possessing endogenous delta opioid receptor expression. In certain embodiments, the nucleic acid encoding the DORP comprises a polynucleotide sequence having at least 80%, 85%, 90%, 95%, 96%, 97%, 98%, or 99% identity to SEQ ID NO: 13 or SEQ ID NO: 14. In certain embodiments, the nucleic acid encoding the DORP comprises the polynucleotide sequence set forth in SEQ ID NO: 13 or SEQ ID NO: 14.

In certain embodiments, the invention also provides an AAV vector comprising a nucleic acid encoding a first region encoding a kappa opioid receptor promoter region capable of being activated when expressed in cells possessing endogenous kappa opioid receptor expression. In certain embodiments, the nucleic acid encoding the KORP comprises a polynucleotide sequence having at least 80%, 85%, 90%, 95%, 96%, 97%, 98%, or 99% identity to SEQ ID NO: 15. In certain embodiments, the nucleic acid encoding the MORP comprises the polynucleotide sequence set forth in SEQ ID NO: 15.

In certain embodiments, the invention also provides an AAV vector comprising a nucleic acid encoding a second or third region encoding an exogenous protein. In certain embodiments, the exogenous protein is a reporter protein. In certain embodiments, the reporter protein is mCherry and is encoded by a polynucleotides sequence having at least 80%, 85%, 90%, 95%, 96%, 97%, 98%, or 99% identity to SEQ ID NO: 12. In certain embodiments, the nucleic acid encoding the mCherry protein comprises the polynucleotide sequence set forth in SEQ ID NO: 12.

In certain embodiments, the reporter protein is eYFP and is encoded by a polynucleotide sequence having at least 80%, 85%, 90%, 95%, 96%, 97%, 98%, or 99% identity to SEQ ID NO: 10. In certain embodiments, the nucleic acid encoding the eYFP protein comprises the polynucleotide sequence set forth in SEQ ID NO: 10.

In certain embodiments, the exogenous protein is a designer receptor exclusively activated by a designer drug (DREADD). In certain embodiments, the DREADD is encoded by a polynucleotide sequence having at least 80, 85%, 90%, 95%, 96%, 97%, 98%, or 99% identity to SEQ ID NO: 11. In certain embodiments, the nucleic acid encoding the DREADD protein comprises the polynucleotide sequence set forth in SEQ ID NO: 11.

In certain embodiments, the nucleic acid of the invention comprises linker sequences which separate the first, second, and third regions of the nucleic acid. In certain embodiments, the linker sequence comprises a polynucleotide sequence having at least 80%, 85%, 90%, 95%, 96%, 97%, 98%, or 99% identity to SEQ ID NO: 8 or SEQ ID NO: 9. In certain embodiments, the linker sequence comprises the polynucleotide sequence set forth in SEQ ID NO: 8 or SEQ ID NO: 9.

TABLE 1
Sequences used in the invention

| SEQ ID | | |
|---|---|---|
| NO: | Name: | Sequence: |
| 1 | Primer | cgcacgcgtgagaacatatggttggacaaaattc |
| 2. | Primer | ggcaccggtggaagggagggagcatgggctgtgag |
| 3. | mMORp- | gagaacatatggttggacaaaattcaggtttgtactctgagaagaagactgaggtaaagtagcatcacactgcgagagattaca |
| | eYFP¹ | gggctagatcacaaaatgatctgatagcaaaggcagcagtaagcaccacaagtcaacctgtcattcacaagaagggggtaa |
| | | agatggttctgtgtgtcctggggagaccgatgaatgtgagcacacagatttcagctcagccatatgaactgacacacttattccga |
| | | agagaatatatttaaaggtattttagcagcctctagaaaaccttatctacattactgctgaaatgacaccctttaacgcaaaaggg |
| | | gacaaataatactgaacgttgtccaaaagtctacaatagttgagctgtgtgttatctttttttcagaatgattgcttaaaagtaaaaat |
| | | atatgttccttctaagcttgcttccccacagtctgtgagaattcagttaaacttctacaaccaaatgggagcggggacatttatttcca |
| | | gtatttatagttaaaactatgggtatgcgcatggcatgcatatgcatacacagtgttgagacgaaaccacactactgctgaatttac |
| | | gattgtattccttcagaagcacttgtgaacttagacatgagatctaacattttttaaatgcattcttgctcctcaaggatcttgcttaag |
| | | gccatttcacactcaaaagagctgtggagtttcttttggattctctatagtgggaagaaaaaacatttctgcttgattcagtaacctgc |
| | | tccccagaagctcgggagagtggaggcagtgcgaaagcttaggataaatcttcctaagcaggtccaggagagtagacctggc |
| | | atgggtagtgggttccttatttacaggttatcacccctcctccctcacatctctaatgtgttcttatgcttcttcttcactaggaaaaaaa |
| | | tcagatgcaaatagaccttctaacctaagagaaattgaaggagtgggggcacatgaaacaggcttctttcacaaaaattttctctt |
| | | ttagaaaaagacaattgtttctttgaacaggtttgtgggggttgattttttgaggaactaaaggggccttttgttttgttttgttttggtttgt |
| | | ttgtttgttttccctcagatatggggatgtgggtaaaggacaacattaataaccctccagtcctaattaattgcatatggttagttgagt |
| | | cggaagagtgttgaggtataagggtcattgagattttaacagaccaacgactttcctggctgatgcaaacaacctcaaacaacct |
| | | aatgcatgttgttacaaagaaacttagagtctctcttcccaccctctctgcagcatcccgcttctgctccccccccccctaccccaatt |
| | | tacactcctttacacggaagtgcatagagactgaggaggctgattctgagttgctttgcgcacaatccactccttctctctcctccct |
| | | cccctctagcctctggatccctcacagcccatgctccctcccttccaccggtgccaccatggtgagcaagggcgaggagctgttca |
| | | ccggggtggtgcccatcctggtcgagctggacggcgacgtaaacggccacaagttcagcgtgtccggcgagggcgagggcgat |
| | | gccacctacggcaagctgaccctgaagttcatctgcaccaccggcaagctgcccgtgccctggcccaccctcgtgaccaccttcgg |
| | | ctacggcctgcagtgcttcgcccgctaccccgaccacatgaagcagcacgacttcttcaagtccgccatgcccgaaggctacgtcc |
| | | aggagcgcaccatcttcttcaaggacgacggcaactacaagacccgcgccgaggtgaagttcgagggcgacaccctggtgaacc |
| | | gcatcgagctgaagggcatcgacttcaaggaggacggcaacatcctggggcacaagctggagtacaactacaacagccacaacg |
| | | tctatatcatggccgacaagcagaagaacggcatcaaggtgaacttcaagatccgccacaacatcgaggacggcagcgtgcagct |
| | | cgccgaccactaccagcagaacacccccatcggcgacggccccgtgctgctgcccgacaaccactacctgagctaccagtccgc |
| | | cctgagcaaagaccccaacgagaagcgcgatcacatggtcctgctggagttcgtgaccgccgccgggatcactctcggcatggac |
| | | gagctgtacaagtaa |
| 4. | mMORp- | gagaacatatggttggacaaaattcaggtttgtactctgagaagaagactgaggtaaagtagcatcacactgcgagagattaca |
| | mDREADD | gggctagatcacaaaatgatctgatagcaaaggcagcagtaagcaccacaagtcaacctgtcattcacaagaagggggtaa |
| | Gi² | agatggttctgtgtgtcctggggagaccgatgaatgtgagcacacagatttcagctcagccatatgaactgacacacttattccga |
| | | agagaatatatttaaaggtattttagcagcctctagaaaaccttatctacattactgctgaaatgacaccctttaacgcaaaaggg |
| | | gacaaataatactgaacgttgtccaaaagtctacaatagttgagctgtgtgttatctttttttcagaatgattgcttaaaagtaaaaat |
| | | atatgttccttctaagcttgcttccccacagtctgtgagaattcagttaaacttctacaaccaaatgggagcggggacatttatttcca |
| | | gtatttatagttaaaactatgggtatgcgcatggcatgcatatgcatacacagtgttgagacgaaaccacactactgctgaatttac |
| | | gattgtattccttcagaagcacttgtgaacttagacatgagatctaacattttttaaatgcattcttgctcctcaaggatcttgcttaag |
| | | gccatttcacactcaaaagagctgtggagtttcttttggattctctatagtgggaagaaaaaacatttctgcttgattcagtaacctgc |
| | | tccccagaagctcgggagagtggaggcagtgcgaaagcttaggataaatcttcctaagcaggtccaggagagtagacctggc |
| | | atgggtagtgggttccttatttacaggttatcacccctcctccctcacatctctaatgtgttcttatgcttcttcttcactaggaaaaaaa |
| | | tcagatgcaaatagaccttctaacctaagagaaattgaaggagtgggggcacatgaaacaggcttctttcacaaaaattttctctt |
| | | ttagaaaaagacaattgtttctttgaacaggtttgtgggggttgattttttgaggaactaaaggggccttttgttttgttttgttttggtttgt |
| | | ttgtttgttttccctcagatatggggatgtgggtaaaggacaacattaataaccctccagtcctaattaattgcatatggttagttgagt |
| | | cggaagagtgttgaggtataagggtcattgagattttaacagaccaacgactttcctggctgatgcaaacaacctcaaacaacct |
| | | aatgcatgttgttacaaagaaacttagagtctctcttcccaccctctctgcagcatcccgcttctgctccccccccccctaccccaatt |
| | | tacactcctttacacggaagtgcatagagactgaggaggctgattctgagttgctttgcccacaatccactccttctctctcctccct |
| | | cccctctagcctctggatccctcacagcccatgctccctcccttccaccggtgccaccatggccaacttcacacctgtcaatggcag |
| | | ctcgggcaatcagtccgtgcgcctggtcacgtcatcatcccacaatcgctatgagacggtggaaatggtcttcattgccacagtgaca |
| | | ggctccctgagcctggtgactgtcgtgggcaacatcctggtgatgctgtccatcaaggtcaacaggcagctgcagacagtcaacaa |
| | | ctacttcctcttcagcctggcgtgtgctgatctcatcataggcgccttctccatgaacctctacaccgtgtacatcatcaagggctactgg |
| | | cccctgggcgccgtggtctgcgacctgtggctggccctggactgcgtggtgagcaacgcctccgtcatgaaccttctcatcatcagc |
| | | tttgaccgctacttctgcgtcaccaagcctctcacctaccctgcccggcgcaccaccaagatggcaggcctcatgattgctgctgcct |
| | | gggtactgtccttcgtgctctgggcgcctgccatcttgttctggcagtttgtggtgggtaagcggacggtgcccgacaaccagtgcttc |
| | | atccagttcctgtccaacccagcagtgacctttggcacagccattgctggcttctacctgcctgtggtcatcatgacggtgctgtacatc |
| | | cacatctccctggccagtcgcagccgagtccacaagcaccggcccgagggcccgaaggagaagaaagccaagacgctggcctt |
| | | cctcaagagcccactaatgaagcagagcgtcaagaagcccccgcccggggaggccgcccgggaggagctgcgcaatggcaag |
| | | ctggaggaggcccccccgccagcgctgccaccgccaccgcgccccgtggctgataaggacacttccaatgagtccagctcaggc |
| | | agtgccacccagaacaccaaggaacgcccagccacagagctgtccaccacagaggccaccacgcccgccatgcccgcccctcc |
| | | cctgcagccgcgggccctcaacccagcctccagatggtccaagatccagattgtgacgaagcagacaggcaatgagtgtgtgaca |
| | | gccattgagattgtgcctgccacgccggctggcatgcgccctgcggccaacgtggcccgcaagttcgccagcatcgctcgcaacc |
| | | aggtgcgcaagaagcggcagatggcggcccgggagcgcaaagtgacacgaacgatctttgccattctgctggccttcatcctcacc |
| | | tggacgccctacaacgtcatggtcctggtgaacaccttctgccagagctgcatccctgacacggtgtggtccattggctactggctct |
| | | gctacgtcaacagcaccatcaaccctgcctgctatgctctgtgcaacgccacctttaaaaagaccttccggcacctgctgctgtgcca |
| | | gtatcggaacatcggcactgccaggcgggcggccgccgtgagcaagggcgaaggataacatggccatcatcaaggagttc |
| | | atgcgcttcaaggtgcacatggagggctccgtgaacggccacgagttcgagatcgacgagggccgcccctac |
| | | gagggcacccagaccgccaagctgaaggtgaccaagggtggccccctgcccttcgcctgggacatcctgtcccctcagttcatg |
| | | tacggctccaaggcctacgtgaagcaccccgccgacatccccgactacttgaagctgtccttccccgaggcttcaagtgggag |
| | | cgcgtgatgaacttcgaggacggaccgtgacccaggactcctccctgcaggacggcgagttcatctacaaggt |
| | | gaagctgcgcggcaccaacttcccctccgacggccccgtaatgcagaagaagaccatgggctgggaggcctcctccgagcgg |
| | | atgtaccccgaggacggcggccctgaagggcgagatcaagcagaggctgaagctgaaggacggcggccactacgacgctga |
| | | ggtcaagaccacctacaaggccaagaagcccgtgcagctgcccggcgcctacaacgtcaacatcaagttggacatcacctcc |
| | | cacaacgaggactacaccatcgtggaacagtacgaacgcgccgagggccgccactccacccatggacgagctgta |
| | | caagtaa |
| 5. | hMORp- | cctgctaacagacatatgaggtgaatattattagcctaccctcgccttttttaagtaatgagaagactgtcatcctgtagggtaaagt |
| | eYFP³ | aacatgtccaaactcatacagctacaaagttacaaagctgatttataaaatgatttgactccaaggtcaggaattattatactgtgt |
| | | cttgtcttccacatgaactaagcacaaaggaactgaatgcaggcagacagatttcagctcaatataagagaattgttacattagtt |
| | | catggaagaatatgttttaaggtatttttgttagtctctaggaaatctctgtaacattttattgtgtaaattatatgctttaatgtaagagg |
| | | ataaaaataatagtgaacattggcaaaatagcctatgattaatagagtttacctatgagttatctgtttctaagataaatgccaaaa |
| | | aataatattggaattaaatgttcctttcaagatcttccctccctgctccctgaaattgcagtgaatttttcaagaccaactgaggacat |
| | | gtattttcaatgtttatggttaaaagatatgtacatgcacagatatatacatgtacagaaatgagaattacttcagaattggtgttaac |
| | | tttagaaaaaaaaagaccaagaacttactcttggtatttacaaatttatttctaaaatagaagcactcatggacttagaagtaaggt |
| | | ataaaattcaaaaacgtatccatgtttctcaaggatcttgttgtaggccactctaattccatatattatgtggcttttcctagaatttttac |
| | | actagaaaacagactgaatgcaaattttgttttgttttaacaaccttcttctcagaagcatatgtctatcgaggaagtcttcagataa |
| | | aaaagataaacaattccaaacaggtctatgagatttaagatgtgaaagatcaacattatctttagttgactttactggatgccaca |
| | | accttctgatttctgtaaccacttcttatgcctcctacccactgaaacaaaatcagaggcaaacagagcttcaccctagaaattgg |
| | | ggaaaatgaggaacaggttttctgcacaaaagtttatttgtttctcatttctttttcagaaaataaaggatcgctgttgttcccaacag |
| | | gtttgtagggaagaaaattggagaaacattattaccttttcttagatgttggcaacggaggcaacaaggactgcaaaagaaaatt |
| | | gtgtgtcccccattcctaaataatcaaaattggcagtagggatggaagagcattggggttttagggctgttagggtttcatcaagcc |
| | | aatgtattccctgccagattttaaggagaaaaaggcgctggaaaattgagtgatgttagccccctttcttatttttcactgctaccaa |
| | | agactaactctatctctctccccaacccttctctccatctccctcctttagatgtgtttgcacagaagagtgcccagtgaagagacct |
| | | actccttggatcgctttgcgcaaaatccaccccttttccctcctccctcccttccagcctccgaatcccgcatggcccacgctcccctc |
| | | caccggtgccaccatggtgagcaagggcgaggagctgttcaccggggtggtgcccatcctggtcgagctggacggcgacgtaaa |
| | | cggccacaagttcagcgtgtccggcgagggcgagggcgatgccacctacggcaagctgaccctgaagttcatctgcaccaccgg |
| | | caagctgcccgtgccctggcccaccctcgtgaccaccttcggctacggcctgcagtgcttcgcccgctaccccgaccacatgaagc |
| | | agcacgacttcttcaagtccgccatgcccgaaggctacgtccaggagcgcaccatcttcttcaaggacgacggcaactacaagacc |
| | | cgcgccgaggtgaagttcgagcgcgacaccctggtgaaccgcatcgagctgaagggcatcgacttcaaggaggacggcaacatc |
| | | ctggggcacaagctggagtacaactacaacagccacaacgtctatatcatggccgacaagcagaagaacggcatcaaggtgaact |
| | | tcaagatccgccacaacatcgaggacggcagcctccagctcgccgaccactaccagcagaacacccccatcggcgacggcccc |
| | | gtgctgctgcccgacaaccactacctgagctaccagtccgccctgagcaaagaccccaacgagaagcgcgatcacatggtcctgct |
| | | ggagttcgtgaccgccgccgggatcactctcggcatggacgagctgtacaagtaa |
| 6. | mMORp | gagaacatatggttggacaaaattcaggtttgtactctgagaagaagactgaggtaaagtagcatcacactgcgagagatt |
| | | acagggctagatcacaaaatgatctgatagcaaaggcagcagtaagcaccacaagtcaacctgtcattcacaagaagg |
| | | gggtaaagatggttctgtgtgtcctggggagaccgatgaatgtgagcacacagatttcagctcagccatatgaactgaca |
| | | cacttattccgaagagaatatatttaaaggtattttagcagcctctagaaaaccttatctacattactgctgaaatgacaccctt |
| | | taacgcaaaaggggacaaataatactgaacgttgtccaaaagtctacaatagttgagctgtgtgttatctttttttcagaatga |
| | | ttgcttaaaagtaaaaatatatgttccttctaagcttgcttccccacagtctgtgagaattcagttaaacttctacaaccaaatg |
| | | ggagcggggacatttatttccagtatttatagttaaaactatgggtatgcgcatggcatgcatatgcatacacagtgttgaga |
| | | cgaaaccacactactgctgaatttacgattgtattccttcagaagcacttgtgaacttagacatgagatctaacattttttaaat |
| | | gcattcttgctcctcaaggatcttgcttaaggccatttcacactcaaaagagctgtggagtttcttttggattctctatagtggg |
| | | aagaaaaaacatttctgcttgattcagtaacctgctccccagaagctcgggagagtggaggcagtgcgaaagcttaggat |
| | | aaatcttcctaagcaggtccaggagagtagacctggcatgggtagtgggttccttatttacaggttatcacccctcctccct |
| | | cacatctctaatgtgttcttatgcttcttcttcactaggaaaaaaatcagatgcaaatagaccttctaacctaagagaaattgaa |
| | | ggagtgggggcacatgaaacaggcttctttcacaaaaattttctcttttagaaaaagacaattgtttctttgaacaggtttgtg |
| | | ggggttgattttttgaggaactaaaggggccttttgttttgttttgttttggtttgtttgtttgttttccctcagatatggggatgtgg |
| | | gtaaaggacaacattaataaccctccagtcctaattaattgcatatggttagttgagtcggaagagtgttgaggtataagggt |
| | | cattgagattttaacagaccaacgactttcctggctgatgcaaacaacctcaaacaacctaatgcatgttgttacaaagaaa |
| | | cttagagtctctcttcccaccctctctgcagcatcccgcttctgctccccccccccctaccccaatttacactcctttacacgg |
| | | aagtgcatagagactgaggaggctgattctgagttgctttgcgcacaatccactccttctctctcctccctcccctctagcct |
| | | ctggatccctcacagcccatgctccctcccttcc |
| 7. | hMORp | cctgctaacagacatatgaggtgaatattattagcctaccctcgccttttttaagtaatgagaagactgtcatcctgtagggta |
| | | aagtaacatgtccaaactcatacagctacaaagttacaaagctgatttataaaatgatttgactccaaggtcaggaattattat |
| | | actgtgtcttgtcttccacatgaactaagcacaaaggaactgaatgcaggcagacagatttcagctcaatataagagaattg |
| | | ttacattagttcatggaagaatatgttttaaggtatttttgttagtctctaggaaatctctgtaacattttattgtgtaaattatatgct |
| | | ttaatgtaagaggataaaaataatagtgaacattggcaaaatagcctatgattaatagagtttacctatgagttatctgtttcta |
| | | agataaatgccaaaaaataatattggaattaaatgttcctttcaagatcttccctccctgctccctgaaattgcagtgaatttttc |
| | | aagaccaactgaggacatgtattttcaatgtttatggttaaaagatatgtacatgcacagatatatacatgtacagaaatgag |
| | | aattacttcagaattggtgttaactttagaaaaaaaaagaccaagaacttactcttggtatttacaaatttatttctaaaatagaa |
| | | gcactcatggacttagaagtaaggtataaaattcaaaaacgtatccatgtttctcaaggatcttgttgtaggccactctaattc |
| | | catatattatgtggcttttcctagaatttttacactagaaaacagactgaatgcaaattttgttttgttttaacaaccttcttctcag |
| | | aagcatatgtctatcgaggaagtcttcagataaaaaagataaacaattccaaacaggtctatgagatttaagatgtgaaaga |
| | | tcaacattatctttagttgactttactggatgccacaaccttctgatttctgtaaccacttcttatgcctcctacccactgaaaca |
| | | aaatcagaggcaaacagagcttcaccctagaaattggggaaaatgaggaacaggttttctgcacaaaagtttatttgtttct |
| | | catttctttttcagaaaataaaggatcgctgttgttcccaacaggtttgtagggaagaaaattggagaaacattattaccttttc |
| | | ttagatgttggcaacggaggcaacaaggactgcaaaagaaaattgtgtgtcccccattcctaaataatcaaaattggcagt |
| | | agggatggaagagcattggggttttagggctgttagggtttcatcaagccaatgtattccctgccagattttaaggagaaaa |
| | | aggcgctggaaaattgagtgatgttagccccctttcttatttttcactgctaccaaagactaactctatctctctccccaaccct |
| | | tctctccatctccctcctttagatgtgtttgcacagaagagtgcccagtgaagagacctactccttggatcgctttgcgcaaa |
| | | atccaccccttttccctcctccctcccttccagcctccgaatcccgcatggcccacgctcccctcc |
| 8. | Linker | accggtgccacc |
| 9. | Linker | gcggccgcc |
| 10. | eYFP | atggtgagcaagggcgaggagctgttcaccggggtggtgcccatcctggtcgagctggacggcgacgtaaacggcca |
| | | caagttcagcgtgtccggcgagggcgagggcgatgccacctacggcaagctgaccctgaagttcatctgcaccaccgg |
| | | caagctgcccgtgccctggcccaccctcgtgaccaccttcggctacggcctgcagtgcttcgcccgctaccccgaccac |
| | | atgaagcagcacgacttcttcaagtccgccatgcccgaaggctacgtccaggagcgcaccatcttcttcaaggacgacg |
| | | gcaactacaagacccgcgccgaggtgaagttcgagggcgacaccctggtgaaccgcatcgagctgaagggcatcga |
| | | cttcaaggaggacggcaacatcctggggcacaagctggagtacaactacaacagccacaacgtctatatcatggccga |
| | | caagcagaagaacggcatcaaggtgaacttcaagatccgccacaacatcgaggacggcagcgtgcagctcgccgacc |
| | | actaccagcagaacacccccatcggcgacggccccgtgctgctgcccgacaaccactacctgagctaccagtccgccc |
| | | tgagcaaagaccccaacgagaagcgcgatcacatggtcctgctggagttcgtgaccgccgccgggatcactctcggca |
| | | tggacgagctgtacaagtaa |
| 11. | DREADD- | atggccaacttcacacctgtcaatggcagctcgggcaatcagtccgtgcgcctggtcacgtcatcatcccacaatcgctat |
| | Gi | gagacggtggaaatggtcttcattgccacagtgacaggctccctgagcctggtgactgtcgtgggcaacatcctggtgat |
| | | gctgtccatcaaggtcaacaggcagctgcagacagtcaacaactacttcctcttcagcctggcgtgtgctgatctcatcata |
| | | ggcgccttctccatgaacctctacaccgtgtacatcatcaagggctactggcccctgggcgccgtggtctgcgacctgtg |
| | | gctggccctggactgcgtggtgagcaacgcctccgtcatgaaccttctcatcatcagctttgaccgctacttctgcgtcacc |
| | | aagcctctcacctaccctgcccggcgcaccaccaagatggcaggcctcatgattgctgctgcctgggtactgtccttcgt |
| | | gctctgggcgcctgccatcttgttctggcagtttgtggtgggtaagcggacggtgcccgacaaccagtgcttcatccagtt |
| | | cctgtccaacccagcagtgacctttggcacagccattgctggcttctacctgcctgtggtcatcatgacggtgctgtacatc |
| | | cacatctccctggccagtcgcagccgagtccacaagcaccggcccgagggcccgaaggagaagaaagccaagacg |
| | | ctggccttcctcaagagcccactaatgaagcagagcgtcaagaagcccccgcccggggaggccgcccgggaggagc |
| | | tgcgcaatggcaagctggaggaggcccccccgccagcgctgccaccgccaccgcgccccgtggctgataaggacac |
| | | ttccaatgagtccagctcaggcagtgccacccagaacaccaaggaacgcccagccacagagctgtccaccacagagg |
| | | ccaccacgcccgccatgcccgcccctcccctgcagccgcgggccctcaacccagcctccagatggtccaagatccag |
| | | attgtgacgaagcagacaggcaatgagtgtgtgacagccattgagattgtgcctgccacgccggctggcatgcgccctg |
| | | cggccaacgtggcccgcaagttcgccagcatcgctcgcaaccaggtgcgcaagaagcggcagatggggcccggg |
| | | agcgcaaagtgacacgaacgatctttgccattctgctggccttcatcctcacctggacgccctacaacgtcatggtcctgg |
| | | tgaacaccttctgccagagctgcatccctgacacggtgtggtccattggctactggctctgctacgtcaacagcaccatca |
| | | accctgcctgctatgctctgtgcaacgccacctttaaaaagaccttccggcacctgctgctgtgccagtatcggaacatcg |
| | | gcactgccaggcgg |
| 12. | mCherry | gtgagcaagggcgaggaggataacatggccatcatcaaggagttcatgcgcttcaaggtgcacatggagggctccgtg |
| | | aacggccacgagttcgagatcgagggcgagggcgagggccgcccctacgagggcacccagaccgccaagctgaag |
| | | gtgaccaagggtggccccctgcccttcgcctgggacatcctgtcccctcagttcatgtacggctccaaggcctacgtgaa |
| | | gcaccccgccgacatccccgactacttgaagctgtccttccccgagggcttcaagtgggagcgcgtgatgaacttcgag |
| | | gacggcggcgtggtgaccgtgacccaggactcctccctgcaggacggcgagttcatctacaaggtgaagctgcgcgg |
| | | caccaacttcccctccgacggccccgtaatgcagaagaagaccatgggctgggaggcctcctccgagcggatgtaccc |
| | | cgaggacggcgccctgaagggcgagatcaagcagaggctgaagctgaaggacggcggccactacgacgctgaggt |
| | | caagaccacctacaaggccaagaagcccgtgcagctgcccggcgcctacaacgtcaacatcaagttggacatcacctc |
| | | ccacaacgaggactacaccatcgtggaacagtacgaacgcgccgagggccgccactccaccggcggcatggacgag |
| | | ctgtacaagtaa |
| 13. | DORp | gggtcttgctgtcatccaggctggagtgcaggggcaagatctcgactcactgcagcctctattttcccagctcaaatgatc |
| | Seq. 1 | ctcccacctcagcctcccaagtagctgcaactagagacacacattaccattcctggctaattttttttttttttttttttttttttttgt |
| | | agagacagggttttgccatgttgcccacgctggtttcgaactcctaggctcaagtgatctgtctgcctcagcctctcaaaat |
| | | gttcggattacaagtgtgtgccaccgtgcccagcctttttcaatttttcttgtagatccaagggatccatgtgcagatttgcca |
| | | cctgggtatattttgtgatgctgaggtttggggtatgaattatcctgtcaactaagttactgagcatagtattcaatggttagtttt |
| | | acacaatacttttttgtgtgaaggtgatgcacaaaaaatccaatgattacacaataaatacatgggaaactattaatatgtaaa |
| | | gaagaagtacgtgatgctactgcataataaggagagctgggctagttggaggtggggggggatcagggaaggtttcac |
| | | aaaggaagtgatatttaagctgaaatttgaggggtaaataacgcttaggctggggtattggcccagccttccgagtggaa |
| | | ggaatagcatgtacaaatgtcctgaggtaggaaaaaacttggcttggagggttagggaggggaccagaattccaataaa |
| | | gtagccctccttcttcctatcccaatttctctctgctggcactagaaaagtttctgagtaccaggccctttgggaaagggaga |
| | | gagttttggcagcaggtgaccacagcaaaaatctttctgataagtccttaggtggttgagtgggaaagccctggggtggg |
| | | gggcttgctgtagttggtagaagtggctggacagcagctaagtggggtggagaaaatggagaagagggtgagcttggg |
| | | aattctggctttgcttttttctgggaaagggtaggagccttgcacatatatggatgattcaagctagagggtgagatgaggg |
| | | agagctccttgtccagccacacacagatccaagagcattgccccagagcctagagctcagaacccctaatattgttccac |
| | | tccagttccctttgggatctttattatccaaaaagtatttcttgagcacctgctgtgaccccagccctctcctggatgcagggg |
| | | tcaatcagacctggtccctttaaggagggagaccagttcggatagaaataattactatatgggataatttagtgctgagcac |
| | | taaatctgtaacatggggacaataataatagaagctacaccaaagtgttggtgcatggataaagtgcttatcacagtgcctg |
| | | gagcaaagtaatgcttggtagaatgttctggaaaggctaggcaccccaagaagaaggatggctctaatctggaatggtta |
| | | ggtcttggaaggggtgtggcaaggatgacttagccaagtgccccccttctatctgctcgcactgcagccagagtgctcctt |
| | | aaggtggtcttcaagagcctgcccaccacctcatcgccccctcacttcctgccttctcctcttcctggcttcctttcagtccct |
| | | ctgcttcctttatccggaatcctcttccctt |
| 14. | DORp | gagacagggttttgccatgttgcccacgctggtttcgaactcctaggctcaagtgatctgtctgcctcagcctctcaaaatgt |
| | Seq. 2 | tcggattacaagtgtgtgccaccgtgcccagcctttttcaatttttcttgtagatccaagggatccatgtgcagatttgccacc |
| | | tgggtatattttgtgatgctgaggtttggggtatgaattatcctgtcaactaagttactgagcatagtattcaatggttagttttac |
| | | acaatacttttttgtgtgaaggtgatgcacaaaaaatccaatgattacacaataaatacatgggaaactattaatatgtaaaga |
| | | agaagtacgtgatgctactgcataataaggagagctgggctagttggaggtggggggggatcagggaaggtttcacaa |
| | | aggaagtgatatttaagctgaaatttgaggggtaaataacgcttaggctggggtattggcccagccttccgagtggaagg |
| | | aatagcatgtacaaatgtcctgaggtaggaaaaaacttggcttggagggttagggaggggaccagaattccaataaagta |
| | | gccctccttcttcctatcccaatttctctctgctggcactagaaaagtttctgagtaccaggccctttgggaaagggagaga |
| | | gttttggcagcaggtgaccacagcaaaaatctttctgataagtccttaggtggttgagtgggaaagccctggggtggggg |
| | | gcttgctgtagttggtagaagtggctggacagcagctaagtggggtggagaaaatggagaagagggtgagcttgggaa |
| | | ttctggctttgcttttttctgggaaagggtaggagccttgcacatatatggatgattcaagctagagggtgagatgagggag |
| | | agctccttgtccagccacacacagatccaagagcattgccccagagcctagagctcagaacccctaatattgttccactcc |
| | | agttccctttgggatctttattatccaaaaagtatttcttgagcacctgctgtgaccccagccctctcctggatgcaggggtca |
| | | atcagacctggtccctttaaggagggagaccagttcggatagaaataattactatatgggataatttagtgctgagcactaa |
| | | atctgtaacatggggacaataataatagaagctacaccaaagtgttggtgcatggataaagtgcttatcacagtgcctgga |
| | | gcaaagtaatgcttggtagaatgttctggaaaggctaggcaccccaagaagaaggatggctctaatctggaatggttagg |
| | | tcttggaaggggtgtggcaaggatgacttagccaagtgccccccttctatctgctcgcactgcagccagagtgctccttaa |
| | | ggtggtcttcaagagcctgcccaccacctcatcgccccctcacttcctgccttctcctcttcctggcttcctttcagtccctct |
| | | gcttcctttatccggaatcctcttccctt |
| 15. | KORp | gaggattgcttgaagatctatatgttgaatatgtgtcctcaaacctttaatagttgcaaagtagttggaagtctttggataatttt |
| | Seq. 1 | aagtgtggccttaaatgtattcttgggacttcatgcccctcttcctattcttcattactggtgtcaaaacgaatggctttgctctg |
| | | ctgtgctcttctgtcttgagatgctgctttgtcataggcctgaaacaacaaagccagcaaggcatgctctagaacctccaag |
| | | aaagaaagctcaagtatgtgttttcatgaggcaaaactgactagtacaccctaaaaaagtttatctgttcaatggatggataa |
| | | aaataaaagaccaggacccaggtatcactaattttataaagcaaagaatgccagtataaggtcatgcagacacaggaacc |
| | | ttagtaactcaaaacacaagagtagatgccaaagctattacttgctggaatctattagagattctgttagaggaagttgaagc |
| | | agtggatattatatctataacctaaactttttaaacatgaaattgtattttaaaacaggcagtattcatgtatattgcattctgtattt |
| | | tttcattagaaatattacacgattgtggttgcttagttatctgtgtgtttagaacgatattatgagtcttttgttatagagttttaatc |
| | | actttctaacacatataaccacttaacaatatttccataatatgaccaaggccaggaagtatttccccactccccagagtaaa |
| | | tcactctttctgaaatggatcataaaggtgcaaatcagaacatgctgtctttagtactaatccttgcagtgcccacctacctag |
| | | tcaataacatctctcctttctcacagaacaaggatagatcgggaagacaacgctgctgagggtggaatgcattcctgtctg |
| | | acagatgtatcaagctagagcctagactggcctctcttctaagctcccagcttagtatccttacacatcaggaacggactca |
| | | ggaaatttagactcagaactgtgacttcacattagacaacaccatttcatcactctgatgtcactgacactgacagctaacat |
| | | cttaaagagaaaatgtatatatatatttatacatgtatatatatatatatagacagaaattaatttattgtgatgaggttcataattc |
| | | tgaggtacagagaaaggaattaacacacagctttttcctcttggcttctgagaggtaagacactacgggtatttactggtca |
| | | atgacttctggaagaagcatttccttcagagaatgattctgctgattcagatttgaatgaaaatgctgtcactctaaacttactg |
| | | ctgtagtatagagaatgatttcagtttccaaacaatattgctgacctatcgtgaacattttgttcccttgtctgaatagtaagaca |
| | | atggtgcactataaaaaaactttccagatgtgggatggagaaaaccagttttatgtactgtggttttgttgtatcaaatataatt |
| | | catcttgatctgttttacttatataatgtactatgatgataagttagtagcatactgccatgcagaaaccatagagtctaattgtc |
| | | ttttggccactgcagatgggtatattttaaaattgaacaccattttgtactttataaaggttaaataattacaattattgatttcttaa |
| | | aaataaacattaaggtatttttttatacagaaaagaaagatacttcaagaatacttaagtcaagaccactgtgaaggattaaa |
| | | atgaatcatcacctctgacatgaaggactccatagtattccagcctgtttgcatattcatctagctattaatctatgctgtattcc |
| | | ttctcatttttatttaacttatttaatcaaagcactgagatagaaccttaatgatgcctgaagtctttccttatgtttctcaataccct |
| | | ggtatctaggaagtccttttaaaatctacacacacaatgagtgcataagaaataggtacctgcaaagcaaaattaattattaa |
| | | gtggctgtcactctcattcacactctgggtttaagttgcagacatggaaggaaatgttactgtttcttattttttagatacacgaa |
| | | g |

¹Italic, mMORP; Bold, Linker; underline, eYFP
²Italic, mMORP; Bold, Linkers; underline, DREADD-Gi; Italic underline, mCherry
³Italic, hMORP; Bold, Linker; underline, eYFP

### Gene Transfer Systems and Adeno-Associated Virus (AAV)

Gene transfer systems, such as those described in the present disclosure, depend upon a vector or vector system to shuttle the genetic constructs into target cells. Methods of introducing a nucleic acid into a cell, including cells of the inner ear, include physical, biological and chemical methods. Physical methods for introducing a polynucleotide, such as RNA, into a host cell include calcium phosphate precipitation, lipofection, particle bombardment, microinjection, electroporation, and the like. RNA can be introduced into target cells using commercially available methods which include electroporation (Amaxa Nucleofector-II [Amaxa Biosystems, Cologne, Germany]), (ECM 830 (BTX) [Harvard Instruments, Boston, Mass.]) or the Gene Pulser II (BioRad, Denver, Colo.), Multiporator (Eppendort, Hamburg Germany). RNA can also be introduced into cells using cationic liposome mediated transfection using lipofection, using polymer encapsulation, using peptide mediated transfection, or using biolistic particle delivery systems such as “gene guns” (see, for example, Nishikawa, et al. Hum Gene Ther., 12(8):861-70 (2001).

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Chol”) can be obtained from Calbiochem-Behring; dimyristyl phosphatidylglycerol (“DMPG”) and other lipids may be obtained from Avanti Polar Lipids, Inc. (Birmingham, AL). Stock solutions of lipids in chloroform or chloroform/methanol can be stored at about −20° C. Chloroform is used as the only solvent since it is more readily evaporated than methanol.

“Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., (1991) Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Currently, the most efficient and effective way to accomplish the transfer of genetic constructs into living cells is through the use of vector systems based on viruses that have been made replication-defective. Some of the most effective vectors known in the art are those based on adeno-associated viruses (AAVs). AAVs are small viruses of the parvoviridae family that make attractive vectors for gene transfer in that they are replication defective, not known to cause any human disease, cause only a very mild immune response, can infect both actively dividing and quiescent cells, and stably persist in an extrachromosomal state without integrating into the target cell's genome. In certain embodiments, the present disclosure provides an AAV vector comprising the nucleic acid construct of the invention.

Regardless of the method used to introduce the nucleic acid into the cell, a variety of assays may be performed to confirm the presence of the nucleic acid in the cell. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the disclosure.

### Methods

In one aspect, the present disclosure includes a method of assessing or measuring or quantifying Opioid Receptor promoter activity in a cell, comprising contacting the cell with a vector comprising the any of the nucleic acids of the present disclosure. In certain embodiments, the opioid receptor is selected from the group consisting of a mu opioid receptor, a delta opioid receptor, and a kappa opioid receptor. In certain embodiments, endogenous transcription factors or regulatory elements which regulate the activity of the endogenous opioid receptor promoter (e.g. mu opioid receptor, delta opioid receptor, and/or kappa opioid receptor) also interact with and drive the activity of the opioid receptor region of the isolated nucleic acid construct (e.g. MORp, DORp, and/or KORp) and result in expression of the exogenous protein encoded by the second or third regions. In certain embodiments, the cell is a cell of the central nervous system (CNS), including a neuron, an astrocyte, a glial cell and the like. In certain embodiments, the exogenous protein is a reporter protein which allows for visualization and/or quantitation of expression activity. Non-limiting examples of reporter proteins are known in the art, and include GFP and GFP-derivates or variants, YFP and YFP-derivates for variants including eYFP, mCherry or other reporters of the mFruits RFP proteins, and an ion flux indicator such as the calcium indicator GCaMP6f or the like. It is anticipated that any reporter or marker system which is convenient for use in CNS cells is able to be used with the nucleic acid constructs of the invention, and that the skill artisan would be able to select an appropriate reporter system would be compatible with the desired readout.

In another aspect, the present disclosure includes a method of providing analgesia to a subject in need thereof, comprising administering to the subject an effective amount of a composition comprising a recombinant AAV vector comprising an isolated nucleic acid comprising a first region encoding a opioid receptor promoter region operably linked to a second region encoding a designer receptor exclusively activated by a designer drug (DREADD), wherein the DREADD is expressed by cells of the central nervous system; and administering to the subject an effective amount of an agonist capable of engaging and activating the DREADD, thereby providing analgesia by mimicking the effects of opioid receptor agonists without the associated toxic and undesirable side-effects on non-MOR expressing cell populations. In certain embodiments, the opioid receptor promoter region is derived from a mu opioid receptor (MORP). In certain embodiments, the opioid receptor promoter region is derived from a delta opioid receptor (DORP). In certain embodiments, the opioid receptor promoter regions is derived from a kappa opioid receptor (KORP). In certain embodiments, the composition further comprises a pharmaceutically acceptable carrier or excipient. In certain embodiments, the AAV vector comprises a capsid protein of a serotype selected from the group consisting of AAV1, AAV2, AAV4, AAV5, AAV6, AAV7, AAV8, AAV9, AAV9P1, PHP.A, PHP.B, Rh8, Rh10, Rh43, Anc80L65, ShH10, ShH10Y, ShH19, Olig001, DJ, and TM6. In certain embodiments, the wherein the AAV vector comprises a capsid protein of serotype AAV1. In certain embodiments, the AAV vector comprises a capsid protein of serotype AAV5. It is understood that the nucleic acid constructs of the invention could be packaged with any AAV vector system which is capable of efficiently delivering it to the desired cell type or tissue.

In certain embodiments, the DREADD is DREADD-Gi or Gi-DREADD, also known as h4MDi or human muscarinic receptor 4, which is activated by the ligand clozapine-N-oxide (CNO) or deschloroclozapine (DCZ). It is also anticipated that the nucleic acid constructs of the invention which comprise MORp, DORp, and/or KORp regions could be used with any DREADD or RASSL system which is capable of interacting with the Gi-type G protein signaling pathways. In certain embodiments, the composition is administered before the administration of the ligand. In certain embodiments, the subject is a mammal. Non-limiting examples of mammalian subjects capable of being treated with the compositions of the invention include, but are not limited to mice, rats, humans, non-human primates, other rodents such as shrews and the like.

In certain embodiments, the AAV vector is administered with an effective amount of an agent that disrupts the blood brain barrier. Such agents are used to facilitate the access of therapeutic agents such as small molecules, viral vectors (e.g., AAV vectors), and other biologic molecules including proteins, antibodies, signaling molecules, and the like to the tissues of the central nervous system (CNS) including the inner ear, a space otherwise separated from the circulatory system by the blood brain barrier (BBB). The BBB comprises a semi-permeable membranous barrier located at the interface between the blood and the CNS tissue and composed of a complex system of endothelial cells, astroglia, pericytes, and perivascular mast cells. Reversible methods for permeabilizing the BBB sufficient to allow the AAV vectors of the disclosure access to the tissues of the inner ear include but are not limited to chemical methods using, for instance, mannitol treatment to induce osmotic changes that allow greater permeability of the BBB, physical methods using focused ultrasound, or engineering of the AAV capsid proteins to better diffuse across the BBB.

### Pharmaceutical Compositions

Pharmaceutical compositions of the present disclosure may comprise as described herein, in combination with one or more pharmaceutically or physiologically acceptable carriers, diluents, adjuvants or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure are preferably formulated for intravenous administration.

Pharmaceutical compositions of the present disclosure may comprise the AAV vector particles as described herein, in combination with one or more pharmaceutically or physiologically acceptable carriers, diluents or excipients. Such compositions may comprise buffers such as neutral buffered saline, phosphate-buffered saline (PBS) and the like; carbohydrates such as glucose, mannose, sucrose or dextran, mannitol; proteins; polypeptides or amino acids such as glycine; antioxidants; chelating agents such as EDTA or glutathione; adjuvants (e.g., aluminum hydroxide); and preservatives. Compositions of the present disclosure can be formulated for a number of administration routes including middle ear injection, round window diffusion, round window injection, oval window injection, labyrinthomy injection, intracochlear electrode or drug delivery system, oral, inhalation, nasal, nebulization, intravenous injection, intramuscular injection, intrathecal injection, intrapleural injection, intracisterna magna injection, subcutaneous injection, and/or transdermal injection. Pharmaceutical compositions of the present disclosure may be administered in a manner appropriate to the disease to be treated (or prevented). The quantity and frequency of administration will be determined by such factors as the condition of the patient, the type and severity of the patient's disease, and the type and functional nature of the patient's immune response to the phage particles, although appropriate dosages may be determined by clinical trials.

The AAV vector particles of the disclosure can be administered in dosages and routes and at times to be determined in appropriate pre-clinical and clinical experimentation and trials. Administration of the AAV vector particles of the disclosure may be combined with other methods useful to treat the desired disease or condition as determined by those of skill in the art.

In certain embodiments, the effective dose range is measured in units known to a person of skill in the art to be suitable for the description of AAV vector particle doses. In some embodiments, the effective dose range for a vaccine or therapeutic compound of the disclosure is measured by transducing units (TU)/kg/dose or genome copies (GC)/kg/dose or particles/kg/dose. In some embodiments, the dosage provided to a patient is between about 10⁶-10¹⁴ TU/kg. In some embodiments, the dosage provided to a patient is between about 10⁶-10¹⁴ GC/kg. In some embodiments, the effective dose range is measured by colony forming units (CFU), 50% tissue culture infectious dose (TCID₅₀), and combinations thereof.

Actual dosage levels of the active ingredients in the pharmaceutical compositions of this disclosure can be varied so as to obtain an amount of the active ingredient that is effective to achieve the desired therapeutic response for a particular patient, composition, and mode of administration, without being toxic to the patient.

The therapeutically effective amount or dose of a compound of the present disclosure depends on the age, sex and weight of the patient, the current medical condition of the patient and the progression of a disease or disorder contemplated in the disclosure.

For humans, a medical doctor, e.g., physician or delegated advanced practice provider, having ordinary skill in the art can readily determine and prescribe the effective amount of the pharmaceutical composition required. For example, the physician or delegated advanced practice provider could start doses of the compounds of the disclosure employed in the pharmaceutical composition at levels lower than that required in order to achieve the desired therapeutic effect and gradually increase the dosage until the desired effect is achieved.

For animals, a medical doctor, e.g., physician or delegated advanced practice provider or veterinarian, having ordinary skill in the art can readily determine and prescribe the effective amount of the pharmaceutical composition required. For example, the physician or delegated advanced practice provider or veterinarian could start doses of the compounds of the disclosure employed in the pharmaceutical composition at levels lower than that required in order to achieve the desired therapeutic effect and gradually increase the dosage until the desired effect is achieved.

In certain embodiments, the compositions of the disclosure are administered to the patient in dosages that range from one to five times per day or more. In other embodiments, the compositions of the disclosure are administered to the patient in range of dosages that include, but are not limited to, once every day, every two, days, every three days to once a week, and once every two weeks. It is readily apparent to one skilled in the art that the frequency of administration of the various combination compositions of the disclosure varies from individual to individual depending on many factors including, but not limited to, age, disease or disorder to be treated, gender, overall health, and other factors. Thus, the disclosure should not be construed to be limited to any particular dosage regime and the precise dosage and composition to be administered to any patient is determined by the attending physical taking all other factors about the patient into account.

Dosage size can be adjusted according to the weight, age, and stage of the disease of the subject being treated. AAV vector particles may also be administered multiple times at these dosages. The AAV vector particles can be administered by using infusion techniques that are commonly known in the art of immunotherapy or vaccinology. The optimal dosage and treatment regime for a particular patient can readily be determined by one skilled in the art of medicine by monitoring the patient for signs of disease and adjusting the treatment accordingly.

The administration of the AAV vector particle compositions of the disclosure may be carried out in any convenient manner known to those of skill in the art. The AAV vector particles of the present disclosure may be administered to a subject by aerosol inhalation, injection, ingestion, transfusion, implantation or transplantation. The compositions described herein may be administered to a subject or patient via middle ear injection, round window diffusion, round window injection, oval window injection, labyrinthomy injection, intracochlear electrode or drug delivery system, oral, inhalation, nasal, nebulization, intravenous injection, intramuscular injection, intrathecal injection, intrapleural injection, intracisterna magna injection, subcutaneous injection, and/or transdermal injection. It should be understood that the method and compositions that would be useful in the present disclosure are not limited to the particular formulations set forth in the examples.

In certain embodiments, the compositions of the disclosure are formulated using one or more pharmaceutically acceptable excipients or carriers. In certain embodiments, the pharmaceutical compositions of the disclosure comprise a therapeutically effective amount of a compound of the disclosure and a pharmaceutically acceptable carrier.

The carrier can be a solvent or dispersion medium containing, for example, saline, buffered saline, water, ethanol, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal and the like. In many cases, it is advisable to include isotonic agents, for example, sugars, sodium chloride, or polyalcohols such as mannitol and sorbitol, in the composition.

Formulations can be employed in admixtures with conventional excipients, i.e., pharmaceutically acceptable organic or inorganic carrier substances suitable for any suitable mode of administration, known to the art. The pharmaceutical preparations can be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They can also be combined where desired with other active agents, e.g., analgesic agents.

The practice of the present disclosure employs, unless otherwise indicated, conventional techniques of molecular biology (including recombinant techniques), microbiology, cell biology, biochemistry, and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and AAV particles of the disclosure, and, as such, may be considered in making and practicing the disclosure.

It should be understood that the method and compositions that would be useful in the present disclosure are not limited to the particular formulations set forth in the examples. The following examples are put forth so as to provide those of ordinary skill in the art with a complete disclosure and description of how to make and use the cells, expansion and culture methods, and therapeutic methods of the disclosure, and are not intended to limit the scope of what the inventors regard as their disclosure.

### EXPERIMENTAL EXAMPLES

The disclosure is further described in detail by reference to the following experimental examples. These examples are provided for purposes of illustration only, and are not intended to be limiting unless otherwise specified. Thus, the disclosure should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present disclosure and practice the claimed methods. The following working examples, therefore, specifically point out the preferred embodiments of the present disclosure, and are not to be construed as limiting in any way the remainder of the disclosure.

The materials and methods used in the examples disclosed here are now described.

Promoter Selection: A 1.9 Kb genomic region immediately upstream of the mouse mu opioid receptor gene (GenBank: AH005396.3) was analyzed for the presence of transcription factors using the PROMO database maintained by the Universitat Politecnica de Catalunya (Farre et al. 2003, 10.1093/nar/gkg605), and the Eukaryotic Promoter Database (EPD) for mmEPDnew, the Mus musculus (mouse) curated promoter database. Based on the results, a 1.5 Kb segment was selected (mMORp) and amplified from mouse genomic DNA using cgcacgcgtgagaacatatggttggacaaaattc (SEQ ID NO: 1) and ggcaccggtggaagggagggagcatgggctgtgag (SEQ ID NO: 2) as the 5′ and 3′ end primers respectively.

Molecular Cloning: All mMORp and hMORp plasmids were constructed on an AAV backbone by inserting either the mMORp or hMORp promoter ahead of the gene of interest using M1uI and AgeI restriction sites. Every plasmid was sequence verified.

Viral Production: All AAVs (serotypes 1, 5, 8, PHP.S, and PHP.eB) were produced at the Stanford Neuroscience Gene Vector and Virus Core. In brief, AAV1 was produced by standard triple transduction of AAV 293 cells (Agilent). At 72 hours post transduction, the cells were collected and lysed by a freeze-thaw procedure. Viral particles were then purified by an iodixanol step-gradient ultracentrifugation method. The iodixanol was diluted and the AAV was concentrated using a 100-kDa molecular mass-cutoff ultrafiltration device. Genomic titer was determined by quantitative PCR of the WPRE element. All viruses were tested in cultured neurons for fluorescence expression prior to use in vivo.

Primary Neuronal Cell Cultures: Primary cultured hippocampal neurons were prepared from P0 Sprague-Dawley rat pups (Charles River). CA1 and CA3 were isolated, digested with 0.4 mg/mL papain (Worthington), and plated onto glass coverslips pre-coated with 1:30 Matrigel (Becton Dickinson Labware). Cultures were maintained in a 5% C02 humid incubator with Neurobasal-A media (Thermo Fisher) containing 1.25% fetal bovine serum (FBS, HyClone), 4% B-27 supplement (Gibco), 2 mM Glutamax (Gibco) and 2 mg/ml fluorodeoxyuridine (FUDR, Sigma), and grown on coverslips in a 24-well plate at a density of 65,000 cells per well.

HEK293 Cell Cultures: HEK293FT cells (Thermo Fisher) were maintained in a 5% C02 humid incubator with DMEM media (Gibco) supplemented with 10% FBS (Invitrogen), and 1× Penicillin-Streptomycin (Invitrogen). They were enzymatically passaged at 90% confluence by trypsinization.

Primary Neuronal Culture Transduction: 2.0 μg plasmid DNA was mixed with 1.875 μl 2 M CaCl₂) (final Ca²⁺ concentration 250 mM) in 15 μL H₂O. To DNA-CaCl₂) we added 15 μL of 2×HEPES-buffered saline (pH 7.05). After 20 min at room temperature (20-22° C.), the mix was added dropwise into each well (from which the growth medium had been removed and replaced with pre-warmed minimal essential medium (MEM) and transduction proceeded for 45-60 min at 37° C., after which each well was washed with 3×1 mL warm MEM before the original growth medium was returned. Neurons were allowed to express transfected DNA for 6-8 days prior to experimentation.

Human iPS Nociceptor and Cardiomyocte Cultures & Transduction: Human iPS cell derived nociceptors and cardiomyocytes cell cultures were produced as described previously. Briefly, iPS cell-derived nociceptors were plated at 5,000/well density and co-cultured with 2,000 glial cells in 384-well plate, while iPS cell-derived cardiomyocytes were plated 10,000/well without co-culture. Cultured cells were transduced by directly adding concentrated AAV viral particles at multiple titers (1×109, 1×10¹⁰, 1×10¹¹, 1×10¹² gc/mL) into well plates. Cells were fed every two days with maintenance medium (Nociceptors: DMEM/F-12 [Glutamax, Cat #10565018], N-2 Supplement [Cat #A1370701], B-27 Supplement [Cat #A3353501], BDNF [Cat #248-BDB-050/CF], GDNF [Cat #212-GD-050/CF], ß-NGF [Cat #256-GF-100/CF], NT-3 [Cat #267-N3-025/CF]; Cardiomyocytes: RPMI 1640 medium [Cat #11835055], 1×B27 supplement with insulin [Cat #17504-044]).

Mice: Adult male C57BL/6J wild type, male Sst-IRES-Cre knock-in and male and female Oprm1^{Cre}:GFP knock-in/knock-out mice (The Jackson Laboratory, stock #00064, #13044, and #035574) of at least 8 weeks of age, and both adult male and female Oprm1^{2A-Cre}:Sun1-sfGFP knock-in mice were used across all studies. Mice were group housed 2-5 individuals per cage, or individually for select experiments, and maintained on a 12-hour reverse light/dark cycle (lights off at 0930 hour and on at 1830 hour) under controlled temperature (˜-20-25° C.) and humidity (˜30-50%) levels. Animals were given ad libitum access to food and drinking water in primary housing and all secondary behavioral testing suites in the vivarium, unless otherwise noted for experimental manipulations.

Rats: Adult male Sprague-Dawley rats (Charles River, strain code #400) were used for viral and histological studies. Rats were individually housed in hanging wire cages and maintained on a 12-hour light/dark cycle under temperature and humidity-controlled conditions. Animals were allowed ad libitum access to food and drinking water in all housing containers.

Shrews: Adult female Asian house shrews (Suncus murinus) were used for viral and histological studies. Shrews were bred and maintained at the University of Pennsylvania and derived from a Taiwanese strain initially supplied to investigators by the Chinese University of Hong Kong. Animals were individually housed in plastic cages (Innovive) and maintained on a 12-hr light/dark cycle under temperature and humidity-controlled conditions, as above. Shrews were similarly granted ad libitum access to food (mixture of 75%, laboratory feline food [5003, Lab Diet) and 25% ferret food [5LI4, Lab Diet]) and drinking water in all housing containers.

Rhesus macaque: A single adult male rhesus macaque (Macaca mulatta), aged five years and weighing 6.3 kg, was utilized for viral and histological studies. Subject was housed in an enclosure exceeding regulatory standards to provide ample room for exercise and was maintained on a 12-hr light/dark cycle in a temperature and humidity control environment with visual and touch access to conspecifics. Subject was provided with ad libitum access to drinking water, food, supplementary fruits and vegetables, and enrichment materials. No deprivations were enforced for the duration of the subject's time in university facilities. Health was monitored daily by veterinary and animal care staff.

### Stereotaxic Surgery & Viral Delivery Procedures:

Mice: Adult mice (˜8 weeks of age) were anesthetized with isoflurane gas in oxygen (initial dose=3%, maintenance dose=1.5%), and fitted into Kofp stereotaxic frames for all surgical procedures. 10 μL Nanofil Hamilton syringes (WPI) with 33G beveled needles were used to intracranially infuse AAVs into the medial prefrontal cortex (mPFC), central nucleus of the amygdala (CeA), the ventral tegmental area (VTA), and the dorsomedial striatum (DMS) at a rate of 100 nL/min. The following coordinates were used, based on the Paxinos and Franklin mouse brain atlas (2019), to target these regions of interest: mPFC (from Bregma, AP=+1.25 mm, ML=±0.25 mm, DV=−2.0 mm), CeA (AP=−1.45 mm, ML=+2.91 mm, DV=−4.7 mm), VTA (AP=−3.2 mm, ML=+0.5 mm, DV=−4.7 mm), DMS (AP=+1.10 mm, ML=±1.25 mm, DV=−3.0 mm). For initial immunohistochemistry and in situ hybridization in vivo validation, mice were bilaterally injected with ˜300 nL of recombinant AAV1-mMORp-eYFP (titer: 6.9×10¹¹ gc/mL) and given a 3-4-week recovery period to allow ample time for viral diffusion and transduction to occur. For cell counting studies, mice were injected with either a mix of recombinant AAV5-mMORp-hM4Di-mCherry (titer: 9.8×10¹¹ gc/mL) and AAV5-hSyn-DIO-eGFP (Addgene, #50457, titer: 1.3×10¹³ vg/mL) at a ratio of 9 ul:1 ul, respectively (in Oprm1-Cre mice) or AAV5-mMORp-hM4Di-mCherry alone (in Oprm1^{2A-Cre}:Sun1 mice), with similar recovery periods allowed for these animals prior to tissue collection.

For spinal cord chemogenetic studies, mice were injected intraparenchymally at four sites (bilaterally at L4 and L5) with 400 nL of AAV1-mMORp-hM4Di-mCherry (titer: 3.1×10¹² gc/mL) to drive transduction of inhibitory DREADD receptor in spinal cord neurons. Using a modified, minimally invasive method for microinjection in the mouse spinal dorsal horn without laminectomy⁹¹, mice were first anesthetized with isoflurane gas in oxygen (initial dose=4%, maintenance dose=2%), the back shaved and wiped with alcohol and povidone-iodine followed by a 2 cm incision made through the skin, and then placed within a modified Kopf stereotaxic frame to stabilize the T13 and L3 spinal column vertebrae. Next, the spinal column was slightly arched upwards by ˜30°, 0.2 mg/kg lidocaine was administered to the overlying muscle, which was then was gently dissected to expose the interspace above and below the T13 vertebrae, allowing direct access to the L3-5 spinal cord dorsal surface. A Nanofil 35G beveled flexible steel needle on a 10 μL syringe (WPI) was connected to the stereotaxic arm and positioned over the midline of the T13 transverse process and moved medial-laterally ±0.3 mm and lowered down −0.25 mm to puncture through the spinal dura and place the tip of the needle in the substantia gelatinosa. The virus was then infused at a rate of 100 nL/min and the needle left in place for ˜5 mins post-injection before retracting and completing the remaining injections. The skin was sutured and wiped with povidone-iodine and an intraperitoneal (i.p.) injection of 5.0 mg/kg meloxicam was administered. Mice were observed for recovery and assessed for proper motor coordination over the next three days, with behaviorally testing then following approx. 5 weeks after surgery.

For fiber photometry studies, mice were injected with ˜300 nL of recombinant AAV1-mMORp-GCaMP6f (titer: 3.69×10¹² gc/mL) in the right CeA, followed immediately by the placement of a fiberoptic implant (˜4.7 mm fiber, Doric Lenses) approximately 0.1-0.2 mm above the DV coordinate of the injection site (same as listed above). After setting the fiberoptic in position, MetaBond (Parkel1) and Jet Set dental acrylic (Lang Dental) were applied to the skull of a mouse to rapidly and firmly fix the fiberoptic in place. In brief, after exposure of the skull, the bone was scored with a scalpel blade to provide grooves for the MetaBond to settle into and provide better adhesion. After lowering the fiberoptic into the craniotomy hole, the skull was cleaned with saline and then fully air dried. The MetaBond reagent was then mixed and liberally applied over the skull and up and along the fiberoptic casing. Once dried, the MetaBond was then covered with a layer of Jet Set acrylamide to create a reinforced head cap, as well as to cover the exposed skin of the incision site. Mice were then given a minimum of 4 weeks to recover and allow for optimal viral spread and transduction throughout the region of interest prior to beginning in vivo calcium signal recordings. For all surgical procedures in mice, meloxicam (5 mg/kg) was administered subcutaneously at the start of the surgery, and a single 1 mL injection of sterile saline was provided upon completion. All mice were monitored for up to two days following surgical procedures to ensure the animals' proper recovery.

For cellular subtype labeling studies, C57BL6/J mice were injected with recombinant AAV8-mMORp-mCherry-IRES-Cre (7.0×10¹¹ gc/mL) mixed with AAV9-hDlx-FLEX-eGFP (Addgene, #83895, titer: 2.2×10¹³ vg/mL) at a ratio of 9 μl:1 μl, respectively, and allowed 3-4 weeks for recovery and for viral transduction to occur throughout the mPFC and S1 regions. For INTRSECT-based labeling, Sst-IRES-Cre mice were injected with a mix of recombinant AAV1-mMORp-FlpO (1.42×10¹¹ gc/mL) and recombinant AAV8-hSyn-CON/FON-eYFP (Addgene, #55650, titer: 2.4×10¹³ vg/mL) at a similar ratio of 9 μl:1 μl, respectively, and given a similar recovery period in order to allow for successful transduction and viral spread to occur prior to tissue collection and processing for histological analysis.

For pan CNS labeling studies, a 30G insulin syringe was loaded with ˜50 μl of recombinant AAV-PHP.eB-mMORp-eYFP virus (titer: 8.6×10¹² gc/mL) was used to perform intravenous (retro-orbital) deliveries. Mice were deeply anesthetized with isoflurane gas mixed in oxygen (4%) and then quickly removed from the induction chamber for the injection procedure. Mice were placed in a prone position on a sterile surgical surface with a small nose cone to maintain anesthesia at a dose of 1.5% isoflurane. Using the index finger and thumb of the non-dominant hand, the skin above and below the right eye was gently drawn back to cause the eye to slightly protrude from the socket. With the dominant hand, the needle was inserted with the beveled side down at a ˜30° angle (relative to the nose) into the medial canthus and through the conjunctival membrane. The total volume of the needle was then injected over a ˜2 second period, after which it was slowly withdrawn, and the mouse allowed to recover. The injected eye was monitored for two days after the injection to ensure no sides of infection or other complications. For pan PNS labeling studies examining the DRGs, intracerebroventricular (i.e.v.) injections were performed on neonatal mouse pups at postnatal day 2-4 using either recombinant AAV-PHP. S-mMORp-eYFP (titer: 1.4×10¹³ gc/ml) or an AAV-PHP.S-CAG-tdTomato (Addgene, #59462-PHP.S, titer: 2.1×10¹³ vg/mL) reporter virus. Pups were cryo-anesthetized for 1-2 minutes and then injected with about 4 ul of either virus into the cerebral lateral ventricles. Six weeks after virus administration, animals were sacrificed and DRG tissue collections were performed as previously described.

Rats: Adult rats (˜8 weeks of age) received an i.p. injected of an anesthetic cocktail (KAX, ketamine 9.0 mg/kg, Butler Animal Health Supply; xylazine 2.7 mg/kg, Anased; acepromazine 0.64 mg/kg, Butler Animal Health Supply) and tested to confirm loss of consciousness before being fitting into a Kofp stereotaxic frame. For histological studies, ˜500 nL of recombinant AAV1-mMORp-eYFP (titer: 6.9×1011 gc/mL) was injected bilaterally into the CeA and VTA via a 10 μL Nanofil syringe fitted with a 33G beveled needle (WPI) at a rate of 50 nL/min. The following coordinates were used, based on the Paxinos and Franklin rat brain atlas (2004) these regions, respectively: CeA (from Bregma, AP=−2.4 mm, ML=±4.4 mm, DV=−8.1 mm) and VTA (AP=−5.4 mm, ML=±0.7 mm, DV=8.2 mm). Metacam was administered subcutaneously immediately after surgery and for two consecutive days following surgery.

Shrews: Adult shrews (˜8 weeks of age) were injected i.p. with KAX cocktail (ketamine 9.0 mg/kg, xylazine 0.27 mg/kg, acepromazine 0.064 mg/kg) and tested to confirm loss of consciousness before being fitted into a modified Kopf stereotaxic frame with the head in a ventroflexed position. A midline incision was made above the atlanto-occipital joint, the muscles were retracted, the joint capsule opened, and the ventral hindbrain was visualized similarly as previously described in rats 96. For histological studies, similarly to what was described above for both mice and rats, ˜300 nL of recombinant AAV1-mMORp-eYFP (titer: 6.9×1011 gc/mL) was bilaterally infused into the AP/NTS via a 10 μL Nanofil syringe fitted with a 33G beveled needle (WPI) at a rate of 50 nL/min using the following coordinates: AP=400 um rostral to the obex, ML=±300 um, DV=300 um below the surface of the hindbrain. Metacam was administered i.p. both immediately after surgery and up to two days post-surgery.

Rhesus macaque: A single, five year old male macaque was utilized for immunohistochemistry and in situ hybridization studies. The subject underwent an initial surgical procedure to implant a dedicated headpost for a fiducial marker array system used in tandem with a MRI-guided neurosurgical system (Brainsight Vet, Rogue Research). MRI images for T1 and gadolinium enhanced T1 were acquired on a 3T scanner (Siemens Tim Trio), and the images generated from this scan were then used to create 3D reconstructions of the skin, skull, brain and vasculature of the subject in the Brainsight software. Injection trajectories were planned using Brainsight to minimize the number of craniotomy holes needed to accommodate all target regions and to avoid both major blood vessels and ventricles. Four targets were selected for focal injections based on reported fMRI responses to capsaicin in macaques and fMRI activity to noxious heat in humans: the prefrontal cortex (PFC), insular cortex (Ins.), amygdala (Amyg.) and medio-dorsal thalamus (MDT). On the day of the first intraparenchymal injection surgery, the subject was sedated with an intramuscular (i.m.) injection of 4 mg/kg ketamine/0.025 mg/kg dexmedetomidine, intubated and maintained under inhaled isoflurane (0.5-2.0%). The analgesic buprenorphine (0.01 mg/kg) was administered subcutaneously (s.c.) preoperatively, with a concurrent dose of dexamethasone-SP (0.5 mg/kg intravenous). Intraoperatively, the subject's vitals were continuously monitored by veterinary care staff, and the anesthetized state maintained by anesthesia veterinary technicians. The surgical site was shaved and aseptically prepped, after which an “L” shaped incision was made over the midline and the rostral aspect of the right hemisphere. The skin was retracted to expose the right temporalis muscle, which was detached from its origin using a blunt tissue elevator to the level of the auricular cartilage. The periosteum was scraped, and craniotomy holes made over each injection site using the Brainsight drilling system. A 26G injection cannula (Rogue Research) attached by polyethylene tubing to a microinfusion system (WPI) was backfilled with sterile mineral oil prior to loading ˜40 μL of the AAV1-hMORp-eYFP virus (titer: 1.17×1012 gc/mL) mixed 1:100 with a 100 mM sterile manganese solution for a final concentration of 1 mM. Virus was infused at all sites of interest at a rate of 200 nL/min, with final injection volumes as follows: 3.5 μL in right anterior insular cortex, 2.0 μL in right amygdala and 3.0 μL in right insular cortex. Upon the completion of surgery, the craniotomy holes were filled with bone wax, the superficial temporal fascia was anchored with 2-0 vicryl using a horizontal mattress pattern to replace the right temporalis muscle in the temporal fossa, and the skin was closed with 3-0 vicryl intradermal pattern. A postoperative manganese-enhanced MRI scan was conducted immediately following the surgery and linearly co-registered with pre-operative scans to assess targeting precision via imaging of the manganese contrast signal. Once completed, isoflurane was tapered, and the animal was extubated once consciousness was regained. Sustained release buprenorphine (0.12 mg/kg) was administered s.c. as well as an additional 0.5 mg/kg Dex-SP, and the animal was returned to its enclosure. Dex-SP was administered on the following taper schedule: 1 mg/kg/day (1 day), 0.5 mg/kg/day (1 day), and 0.25 mg/kg/day (1 day). Activity and behaviors were monitored closely over several days following each procedure, with daily observations thereafter to ensure no adverse effects of the surgery on the animal's observable wellbeing. A second intraparenchymal brain injection surgery was also conducted ˜3 months after the first, using the same approach described above, with the following exceptions. The “L” shaped incision was made over the midline and the rostral aspect of the left hemisphere, in order to target the left insula, mediodorsal thalamus and amygdala. The insula and mediodorsal thalamus were injected with 3.0 μL and 4.5 μL of the AAV1-hMORp-eYFP virus (titer: 1.17×1012 gc/mL), respectively, and the left amygdala was injected with 4.5 μL of the AAV1-mMORp-eYFP virus (titer: 1.17×1012 gc/mL).

### RNAscope In Situ Hybridization:

Mice: Animals were anesthetized using isoflurane gas in oxygen, and the brains were quickly removed and fresh frozen in O.C.T. using Super Friendly Freeze-It Spray (Thermo Fisher Scientific). Brains were stored at −80° C. until cut on a cryostat to produce 16 m coronal sections of the mPFC, CeA and VTA. Sections were adhered to Superfrost Plus microscope slides, and immediately refrozen before being stored at −80° C. Following the manufacturer's protocol for fresh frozen tissue for the V2 RNAscope manual assay (Advanced Cell Diagnostics), slides were fixed for 15 min in ice-cold 10% NBF and then dehydrated in a sequence of ethanol serial dilutions (50%, 70%, and 100%). Slides were briefly air-dried, and then a hydrophobic barrier was drawn around the tissue sections using a Pap Pen (Vector Labs). Slides were then incubated with hydrogen peroxide solution for 10 mins, washed in distilled water, and then treated with the Protease IV solution for 30 min at room temperature in a humidified chamber. Following protease treatment, C1 and C2 cDNA probe mixtures specific for mouse tissue were prepared at a dilution of 50:1, respectively, using the following probes from Advanced Cell Diagnostics: EYFP (C1, 312131), Oprm1 (C2, 315841-C2). Sections were incubated with cDNA probes (2 hours), and then underwent a series of signal amplification steps using FL v2 Amp 1 (30 min), FL v2 Amp 2 (30 min) and FL v2 Amp 3 (15 min). 2 min of washing in 1×RNAscope wash buffer was performed between each step, and all incubation steps with probes and amplification reagents were performed using a HybEZ oven (ACD Bio) at 40° C. Sections then underwent fluorophore staining via treatment with a serious of TSA Plus HRP solutions and Opal 520 and 620 fluorescent dyes (1:5000, Akoya Biosystems, FP1487001KT, FP1495001KT). All HRP solutions (C1-C2) were applied for 15 mins and Opal dyes for 30 mins at 40° C., with an additional HRP blocker solution added between each iteration of this process (15 mins at 40° C.) and rinsing of sections between all steps with the wash buffer. Lastly, sections were stained for DAPI using the reagent provided by the Fluorescent Multiplex Kit. Following DAPI staining, sections were mounted, and cover slipped using Aqua-Poly Mount and left to dry overnight in a dark, cool place. Sections from all three regions were collected in pairs, using one section for incubation with the cDNA probes and another for incubation with a probe for bacterial mRNA (dapB, ACD Bio, 310043) to serve as a negative control.

For dorsal root ganglia (DRG) dissection studies, dissections were performed as previously reported 99 with minor modifications. In brief, mice were anesthetized with isoflurane gas for ˜5 min and decapitated, after which the animals were perfused with 0.1 M PBS to remove all blood. Skin and muscles were removed from the back and around the spinal cord (SC). The vertebral columns of SC were exposed and placed on a tray of ice after removal. DRGs were exposed after roughly half of the vertebral columns were removed and the surrounding tissue cut away. Finally, collected DRGs were fixed in 4% PFA for 24 h at 4° C., after which they were cryo-protected in a 30% sucrose dissolved in 0.1M PBS for 24 h at 4° C. DRG were frozen in O.C.T and then stored at −80° C. until ready for sectioning. DRG were sectioned at a thickness of 14 μm on a cryostat and mounted on slides. Sections then underwent RNAscope FISH using the manufacturer's V1 kit and associated protocol (Advanced Cell Diagnostics). Transcript species of interest were detected using the following probes from the manufacturer: tdTomato (C2, 317041-C2), eYFP (C2, 312131-C2), Rbfox3 (C3, 313311-C3), and Oprm1 (C1, 493251).

Rhesus macaque: The tissue preparation process for generating the macaque brain slices used in RNAscope experiments is described in the section above. 16 μm coronal sections of the PFC were adhered to Superfrost Plus microscope slides and immediately refrozen before being stored at −80° C. Following the manufacturer's protocol for fixed frozen tissue for the V2 RNAscope manual assay, slides were washed in 0.1 M PBS to remove excess O.C.T., baked in the HybEZ oven for 30 mins at 60° C., and then post fixed for 15 min in ice-cold 10% NBF. Sections were then dehydrated in a sequence of ethanol serial dilutions (50%, 70%, and 100%), briefly air-dried, and then incubated with hydrogen peroxide solution for 10 mins. Sections were washed in distilled water, and then were subjected to an antigen retrieval step. Utilizing a steamer (Hamilton Beach), sections were submerged in distilled water warmed to ˜99° C. for 10 secs and then switched into a container of 1× Target Retrieval Reagent for 5 mins at ˜99° C. Sections were then cooled in room temperature distilled water for 15 secs before being submerged in 100% EtOH for 3 mins, and then finally dried in the HybEZ over at 60° C. for 5 mins. Hydrophobic barriers were drawn around the tissue sections using a Pap Pen (Vector Labs), and after drying for 5 mins, all sections were incubated at 40° C. for 30 min after treatment with several drops of Protease III solution. Following protease treatment, C1 and C2 cDNA probe mixtures specific for macaque tissue were prepared at a dilution of 50:1, respectively, using the following probes: EYFP (C1, ACD Bio, 312131), OPRM1 (C2, ACD Bio, 518941-C2). The remainder of the RNAscope protocol then proceeded as described above for mouse tissue processing. As for the mouse tissue samples, sections of macaque tissue from all regions of interest were collected in pairs, using one section for incubation with the cDNA probes and another for incubation with a probe for bacterial mRNA (dapB, ACD Bio, 310043) to serve as negative controls.

Imaging and quantification: All mouse, rat, shrew and macaque tissue expressing AAV1-mMORp1-eYFP, AAV5-mMORp-hM4Di-mCherry or AAV1-hMORp-eYFP processed for IHC and FISH were imaged on a Keyence BZ-X all-in-one fluorescent microscope at 48-bit resolution using the following objectives: PlanApo-λ×4, PlanApo-λ×20 and PlanApo-λ×40. Co-localization of eYFP or mCherry signal to neurons stained with antibodies amplified signal or cDNA probes of interest was achieved by adjusting the exposure time and overall gain for the green, red, far red and blue channels to visualize cells with both high and low signal in ×4 magnified snapshots and ×20 and ×40 magnified Z-stack images while correcting for oversaturation. Similar metrics and equipment were used when imaging mouse tissue from use case experiments expressing all other constructs to validate overall viral transduction efficacy. All image processing prior to quantification was performed with the Keyence BZ-X analyzer software (version 1.4.0.1). Imaging for mouse tissue expressing AAV.PHP.S-mMORp-eYFP and processed for FISH was performed with a Zeiss LSM 710 confocal microscope using a 20× objective for cell quantification and a 40× oil-immersion objective for viewing transcript markers at high resolution. Human iPS cells treated with AAV.PHP.S-mMORp-eYFP and processed for IHC were imaged using a Zeiss LSM 710 confocal microscope via a 10× objective and a 20× objective for higher magnification.

Quantification of viral efficacy and selectivity in IHC processed tissue from mice was performed via manual counting of TIF images in Fiji (ImageJ, 2.3.0/1.53q) using the Cell Counter plugin or Photoshop (Adobe, 2021) using the Counter function. Counts were made using ×20 magnified z-stack images of a designated regions of interest (ROI) around injection sites from both the left and right hemispheres and reported as total cells in a given ROI positive for mCherry and anti-Cre signal co-localized to the same cell body, as denoted by DAPI staining. Total counts for these populations were averaged across all hemispheres with positive viral transduction within targeted ROIs from 1 male and 2 female mice for the CeA and VTA, 1 male and 1 female for the mPFC, and 1 male and 1 female for the DMS in the Oprm1-Cre:GFP line, and 2 male mice for the CeA and VTA, 1 male for the mPFC and 2 females for the DMS in the Oprm1-Cre:Sun1 line. For FISH images, TIFs of tissue sections treated with the dapB negative control probe for each pair of slides were used to determine brightness and contrast parameters that minimized observation of bacterial transcripts and auto fluorescence, and these adjustments were then applied to images from the experimental sections which were treated with the cDNA probes. Adjusted experimental images were then analyzed within 4 separate, 20×ROIs within macaque sections taken from the PFC. Cells in these ROIs were identified using DAPI-stained nuclei, and the total number of cells in each region were counted. Cells were then appraised for the presence of EYFP and OPRM1 transcript signal to determine the total number of cells labeled for these probes either alone or in combination. Transcripts were readily identified as round, fraction delimited spots over and surrounding DAPI-labeled nuclei. Total counts for the number of cells showing co-localized signal were summated across the 4 ROIs, and replicate counted was performed and by 4 separate individuals Total counts of all ROIs from each experimenter were averaged and the resulting values reported. FISH images taken of mouse DRG sections were quantified manually in Fiji using ROIs to define the quantified area. AAV+ and Marker+/AAV+ neurons were counted as those with more than five dots (or transcripts) per ROI, and counts were confirmed as reasonable estimates by comparison to counts labeled with nuclei marker DAPI and pan neuron marker Rbfox3.

Behavioral Testing: All experiments took place during the dark phase of the cycle (0930 hour to 1830 hour). Group and singly housed mice were allowed a 1-2-week acclimation period to housing conditions in the vivarium prior to starting any behavior testing. Additionally, three to five days before the start of testing, mice were handled daily to help reduce experimenter-induced stress. On test days, mice were brought into procedure rooms ˜1 hour before the start of any experiment to allow for acclimatization to the environment. Mice were provided food and water ad libitum during this period. For multi-day testing conducted in the same procedure rooms, animals were transferred into individual “home away from home” secondary cages ˜1 hour prior to the start of testing and were only returned to their home cages at the end of the test day. All testing and acclimatization were conducted under red light conditions (<10 lux), with exposure to bright light kept to a minimum to not disrupt the animals' reverse light cycle schedule. Equipment used during testing was cleaned with a 70% ethanol solution before starting, and in between, each behavioral trial to mask odors and other scents.

Von Frey filament touch test: To evaluate mechanical reflexive sensitivity, we used a logarithmically increasing set of 8 von Frey filaments (Stoelting), ranging in gram force from 0.07 to 6.0 grams. Mice were placed on a metal hexagonal-mesh floored platform (24 in ×10 in) within a transparent red cylinder (3.5 in ×6 in). The filaments were then applied perpendicular to the left plantar hind paw with sufficient force to cause a slight bending of the filament. A positive response was characterized as a rapid withdrawal of the paw away from the stimulus within 4 secs. Using the Up-Down statistical method¹⁰⁰, the 50% withdrawal mechanical threshold scores were calculated for each mouse and then averaged across the experimental groups. For the two rounds of inhibitory chemogenetic testing conducted using this assay, mice were administered a dose of either the drug DCZ intrathecally at a dose of 10 pg (first round, 5 weeks post-surgery) or CNO i.p. at a dose of 3 mg/kg by body weight (second round, 8 weeks post-surgery, 3 weeks post-DCZ trials), with animals being returned to their home cages for ˜30 mins after injections to allow for complete absorption of drugs prior to the start of any behavioral testing.

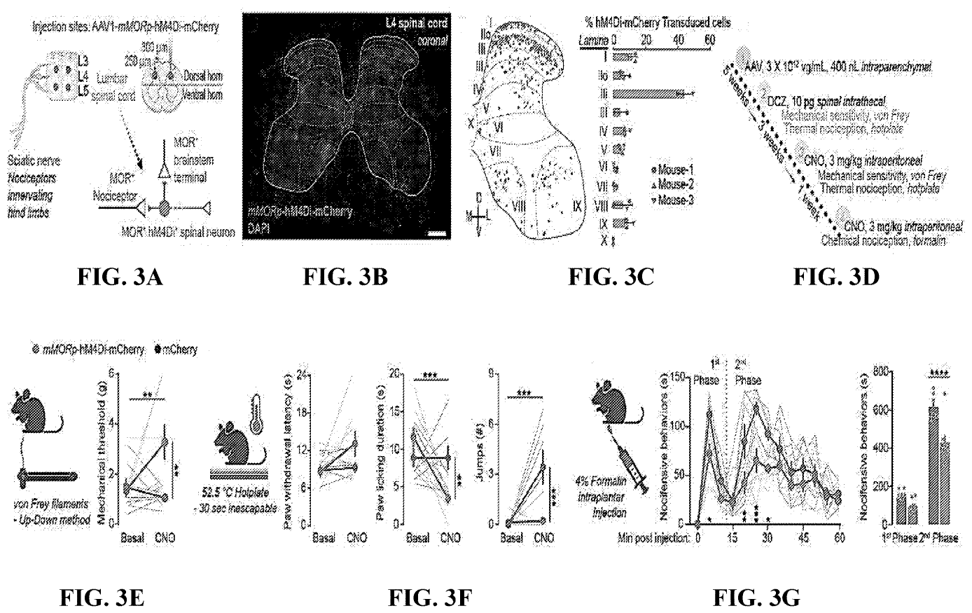

Hotplate thermal tests (static & dynamic): To evaluate thermal sensitivity, we used an inescapable hotplate set to ˜50-52.5° C. The computer-controlled hotplate (6.5 in×6.5 in floor, Bioseb) was surrounded by a 15 in high clear plastic chamber and a web camera was positioned at the front of the chamber to continuously record animals to use for post hoc behavioral analysis. For the static temperature tests conducted for chemogenetic inhibition studies, mice were administered either a 10 pg dose of DCZ or a 3 mg/kg dose of CNO (FIG. 3c), and then returned to their home cages for ˜30 mins to allow for complete absorption of drugs prior to the start of behavioral testing. Mice were then gently placed on the hotplate floor and removed from the chamber after 30 secs. Behaviors were scored in real-time for the latency to the first paw withdrawal, the total duration of paw licking, and the total number of jumps/escape events observed over the entire trial period.

Formalin chemical test: To evaluate chemical induced nocifensive responses, mice received an intraplantar 10 μL injection of a 4% formalin solution. Mice were lightly restrained in a prone position with the left hind limb held between the thumb and fore finger of the non-dominant hand, while a formalin-loaded glass Hamilton syringe with a 30G needle was held in the dominant hand. The needle was inserted in the middle of the left hind paw at a ˜30° angle and the formalin solution was injected over a ˜2 sec period. Next, the mice were quickly placed within transparent red cylinder on a stage with a clear plastic floor, with a mirror angled at 45° underneath, while a web camera recorded behavior from both underneath and to the side of the apparatus for 60 mins. These videos were then blind scored in 2-min bins for reflexive paw flinches, paw guarding, paw licking and jumps. Time course data was summed into the classically defined phases of the formalin test¹⁰¹: 1^{st} phase, interphase and 2^{nd} phase, which reflect different engagements of peripheral and central sensitization mechanisms. For all chemogenetic inhibitory studies, mice were administered an i.p. injection of CNO (3 mg/kg body weight), after which they were returned to their home cage for ˜30 mins prior to the start of testing to allow for full absorption of the drug to occur.

Air puff stimulation: Response to an unexpected stimulation was assessed during fiber photometry testing to validate the overall success of viral transduction of CeA neurons in mice. Mice were placed in transparent red cylinders on top of a metal hexagonal-mesh floored platform as described above, with a web camera position to record them continuously during testing. Using a canister of compressed air (Century Cleaning Duster), a ˜1 sec long blast was applied to the underside of the animal to elicit a startle response. This process was repeated 10 times, with an inter stimulation interval of ˜1 min. Behavioral responses time-locked to calcium mediated events were scored post-hoc using the acquired video footage.

Hot water hindpaw stimulation test: To evaluate responses to acute, noxious thermal stimulations during fiber photometry testing, we used a hot water hind paw application protocol described previously¹⁰². In brief, animals were placed in transparent red cylinders placed on top of a metal hexagonal-mesh floored platform, while a small hot plate was used to heat distilled water to ˜70-72° C. Using a 1 mL syringe, a drop of the hot water was quickly applied to the underside of the left plantar hind paw (temp=55-57° C. at time of application). This process was repeated for a total of 10 applications, with each droplet applied at a 1 min interval. Animals were continuously recorded by a web camera positioned to face the front of the cylinder in which the animal was housed, and all relevant behaviors time-locked to calcium mediated events were scored post-hoc using the video footage. Responses to these noxious stimuli were also tested following acute i.p. administration of morphine (˜10 mg/kg body weight). After injection, animals were placed back in their home away from home cages for 30 mins to allow for complete absorption of the drug. Hot water hind paw stimulation testing then proceeded as described above in the naïve condition.

Chronic morphine drinking & precipitated withdrawal testing: Withdrawal behaviors were assessed during fiber photometry studies by use of a forcing morphine drinking paradigm in mice, achieved by substituting their drinking water for glass stopper bottles (Braintree Scientific) containing morphine or saccharin laced water. Morphine drinking animals were supplied with a 0.3 mg/mL morphine, 0.2% saccharin solution for 3 days, followed 0.5 mg/mL morphine, 0.2% saccharin for 4 days prior to inducing precipitated withdrawal with an i.p. injection of naloxone hydrochloride. Control animals were supplied with 0.2% saccharin laced water only for the duration of testing. After the final day of forced drinking, animals were injected with 3 mg/kg body weight naloxone and then immediately placed in a 15 in high plastic container on top of a metal hexagonal-mesh floored platform. Two web cameras were set up to continuously record the animals from separate angles, and withdrawal related behaviors (wet dog shakes, paw tremors, jaw tremble, diarrhea, etc.) were assessed for ˜20 mins, with total calcium-mediated events used as a read out for overall change in CeA activity in response to naloxone injection.

In Vivo Fiber Photometry Recordings and Analysis: Optical recordings of GCaMP6f fluorescence were acquired using an RZ10× fiber photometry detection system (Tucker-Davis Technologies), consisting of a processor with Synapse software (Tucker-Davis Technologies), and optical components (Doric Lenses and ThorLabs). Excitation wavelengths generated by LEDs (460 nm blue light and 405 nm violet light) were relayed through a filtered fluorescence minicube at spectral bandwidths of 460-495 and 405 nm to a pre-bleached mono fiberoptic patch cord connected to the implant on top of each animal's head. Power output for the primary 460 nm channel at the tip of the fiberoptic cable was measured at ˜25-30 mW. Single emissions were detected using a femtowatt photoreceiver with a lensed cable adapter. Signal in both 460 and 405 nm channels were monitored continuously throughout all recordings, with the 405 nm signal used as an isosbestic control for both ambient fluorescence and motion artifacts introduced by movement of the fiberoptic implant. Wavelengths were modulated at frequencies of 210-220 and 330 Hz, respectively, and power output maintained at a range of ˜20-60 mA for the 460 channel and ˜20 mA for the 405 channel, with a DC offset of 3 mA for both light sources. All signals were acquired at 1 kHz and lowpass filtered at 3 Hz. Mice were housed and handled as described above, with the addition of a 5 min session each handling day during which the mice were hooked up to the fiberoptic patch cord to allow them to become accustomed the tethered cable. On testing days, mice were connected to the photometry system, and following a 1-2 min habituation period, mice were placed into the respective equipment for each of the test described above. Following testing, all mice were perfused and the tissue was assessed for proper viral targeting and transduction efficacy, as well as optic fiber placement via immunohistochemistry (see above section for details).

Analysis of the GCaMP signal was performed with the use of the open source, fiber photometry analysis MATLAB software suite, pMAT103. Using pMAT, bulk fluorescent signal from both the 460 and 405 channels were normalized to compare differences in calcium-mediated event metrics for both the total duration of a recording (frequency) and at select events using peri-event time histogram (PETH) analyses locked to specific behaviors designated by the application of an external transistor-transistor logic (TTL) input (amplitude and area under the curve) across groups, with the 405 channel serving as a control signal. Linear regression was used to correct for the bleaching of signal for the duration of each recording, using the slope of the 405 nm signal fitted against the 460 nm signal. Detection of GCaMP-mediated fluorescence is presented as a change in the 460 nm/fitted 405 nm signal over the fitted 405 signal (ΔF/F). Peak analysis of calcium-mediated events to determine frequency in precipitated withdrawal testing was performed by running the normalized, filtered signals generated by pMAT through Clampfit 10.6 software and performing threshold matched event detection analyses. The threshold value was set to examine the amplitude of an imputed, positive going “spikes” that exceeded the baseline noise of the combined ΔF/F signal (typically set to exceed a value of 1-2).

Drugs and Delivery: For chemogenetic studies, deschloroclozapine (DCZ dihydrochloride, water soluble; HelloBio, HB9126) was delivered intrathecally at a dose of 10 μg in 5 μL saline, and clozapine N-oxide (CNO dihydrochloride, water soluble; HelloBio HB6149) was delivered i.p. at a dose of 3.0 mg/kg body weight. For fiber photometry behavioral testing, morphine sulfate (Hikma) was delivered acutely i.p. at a dose of 10 mg/kg body weight, or chronically at concentrations of 0.3 mg/mL and 0.5 mg/mL by mixing into drinking water along with 0.2% saccharin sodium hydrate (Sigma) in the animals' drinking water. Precipitated withdrawal was inducing during photometry studies by administering naloxone hydrochloride (HelloBio, HB2451) i.p. at a dose of 3 mg/kg body weight.

Statistical Analyses: The number of animals used in each experiment were predetermined based on analyses of similar experiments in the literature and supplemented as needed based on observed effect sizes. All data are presented as mean±the standard error of the mean (SEM) for each group, and all statistical analyses were performed using Prism 9 software (GraphPad Software). We used primarily male mice for all relevant histological cell counting analyses, as well as all chemogenetic and fiber photometry based behavioral studies, and thus did not attempt to assess sex related differences in these use case experiments. Female shrews were used in cross species histological studies, but as those were primarily qualitative evaluations of the efficacy of our viruses, and no meaningful statistical analysis was performed.

For all IHC-based quantification of virally transduced cells in either OprmCre or Oprm12A-Cre:Sun1 mice, total cell counts are presented as either parts of a whole for comparing mMORp-mCherry/anti-Cre+vs. mMORp-mCherry/anti-Cre− stained populations, and summary statistical data for total cell counts and groupings based on transgene reporter or anti-Cre signal are reported as well. Similar statistical analyses and reporting were performed for ISH-based quantification of macaque tissue sections as well. ISH-based analyses of total transduced cells within mouse DRG following AAV-PHP.s-mMORp-eYFP injections were conducted via unpaired two-tailed Student's t-tests, and reported as total Oprm1+/eYFP+ out of all Oprm1+ cells quantified. For behavior experiments, data comparing all metrics assessed between mMORp-hM4Di+ vs. mCherry+ controls was analyzed using repeated-measures two-way ANOVAs along with Bonferonni multiple comparison post hoc tests. Corrected P values are reported in the text as needed. For comparing behavioral results from mMORp-GCaMP+ vs. mMORp-eYFP+ controls across tasks, two-tailed unpaired t-test analyses were performed on data regarding the frequency of imputed calcium-mediated events, changes in the amplitude values, and computed area under the curve (AUC) for select time-locked events from in vivo fiber photometry recordings (all of which consisted of comparisons between only two groups/conditions), with corrected P values presented in the text. For all analyses, significance levels were set at an alpha of 0.05. Detailed statistics are provided within the text and drawing descriptions.

### Example 1: Designing Viral Constructs to Target Promoter Sequences within the Oprm1 Gene

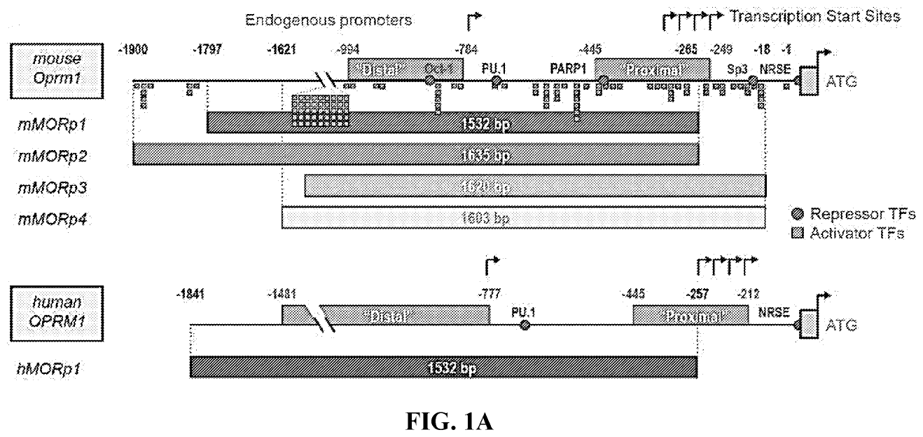

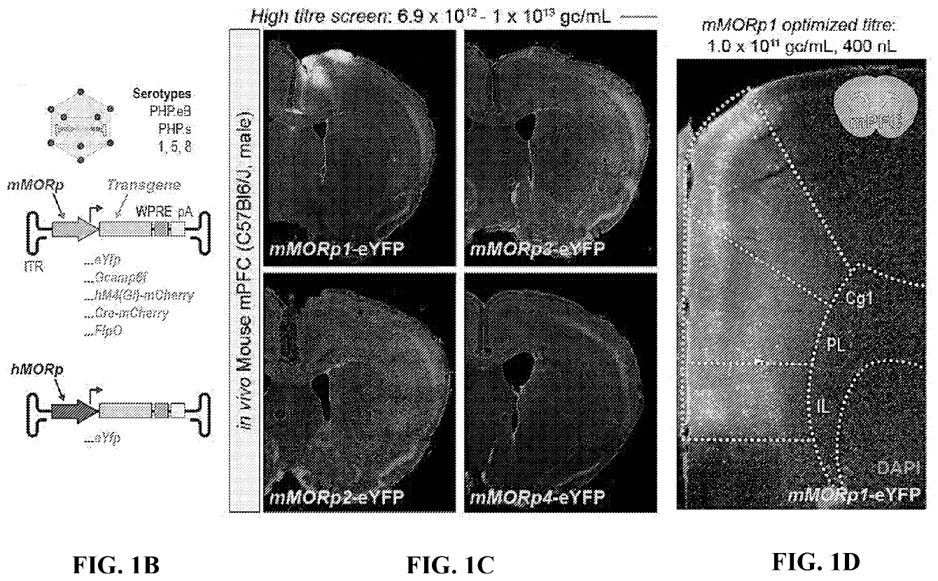

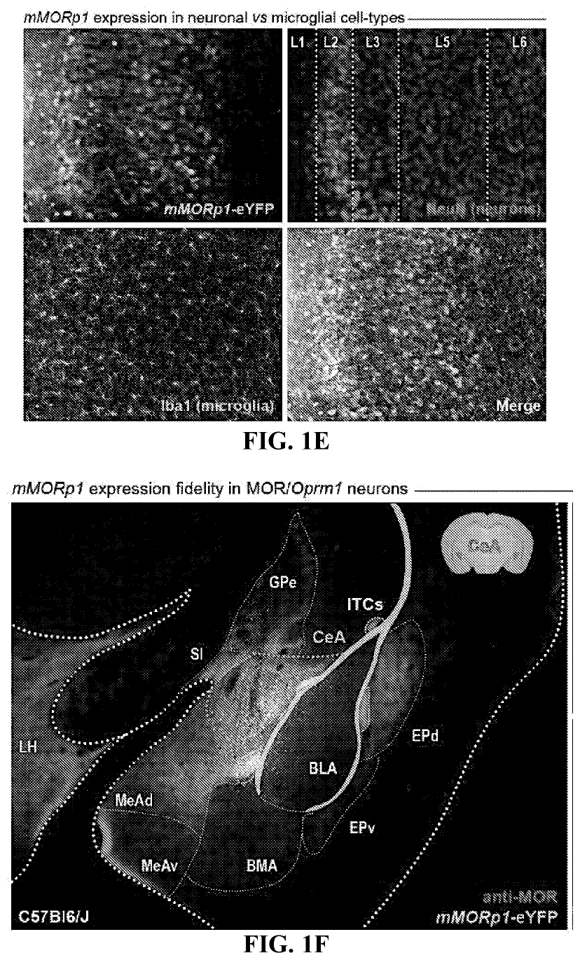

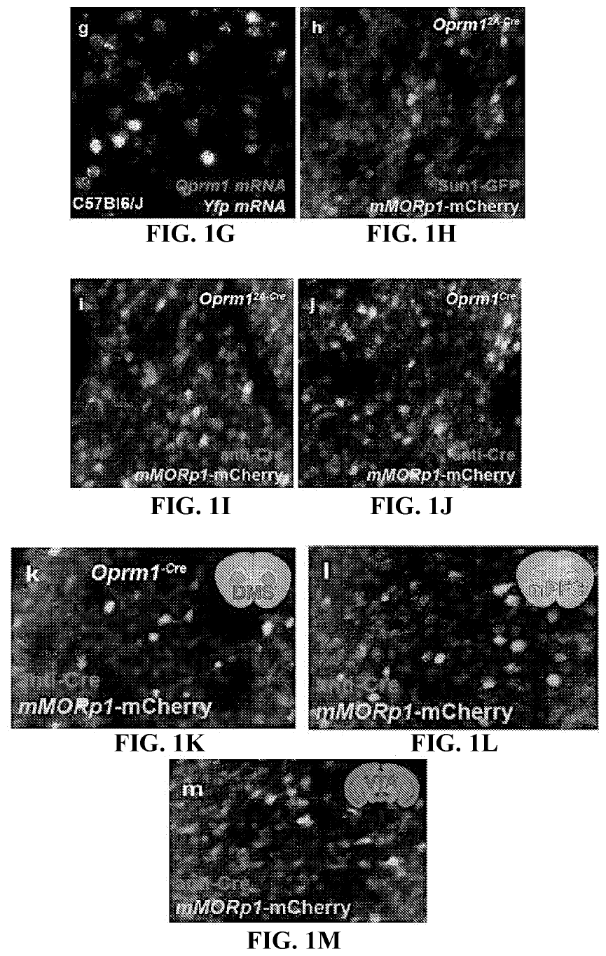

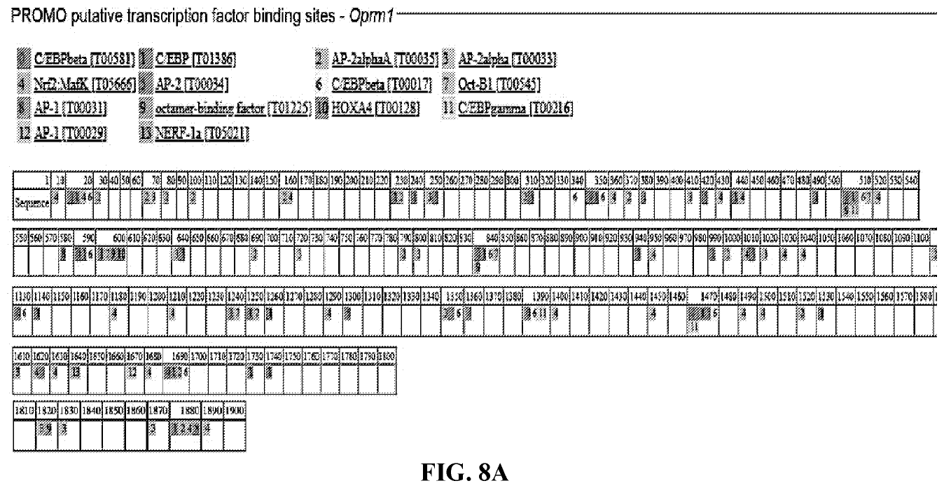

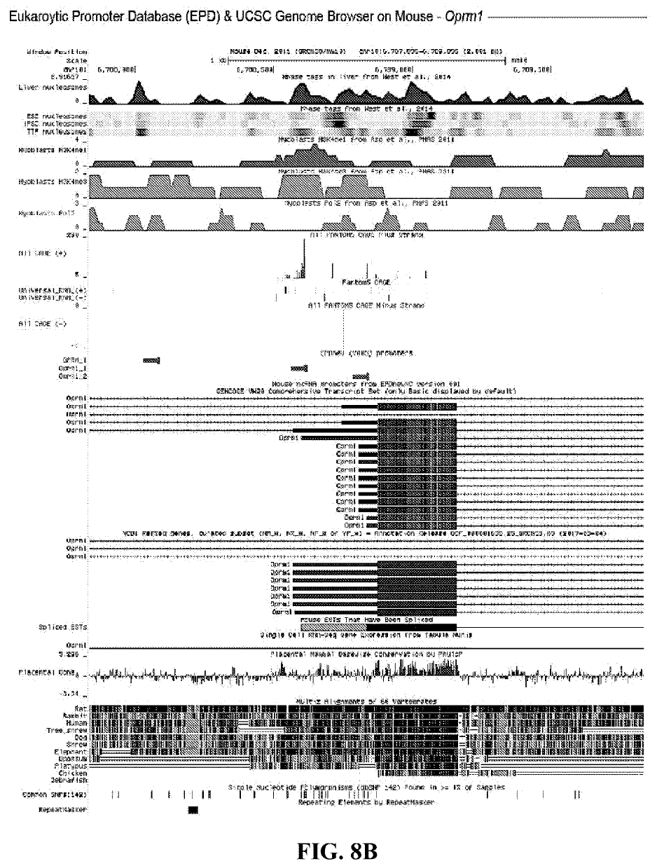

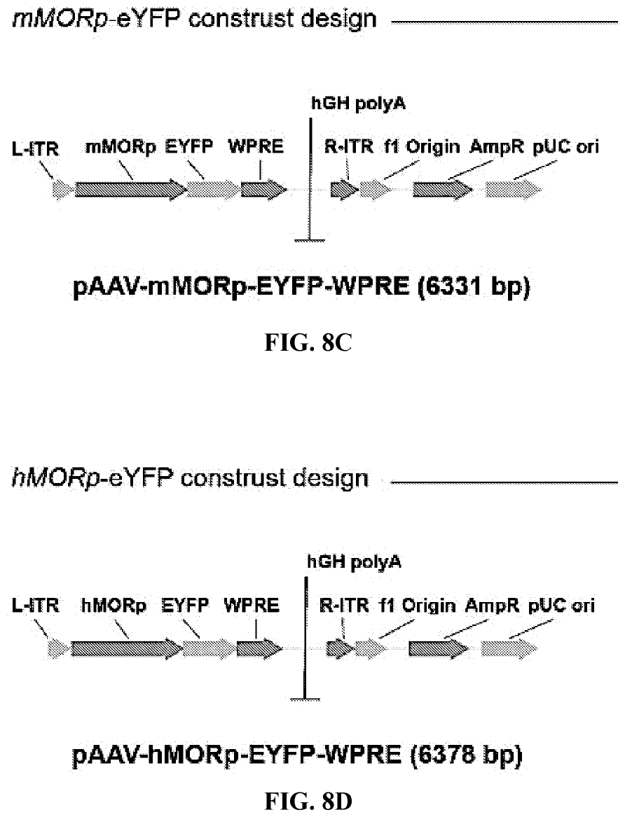

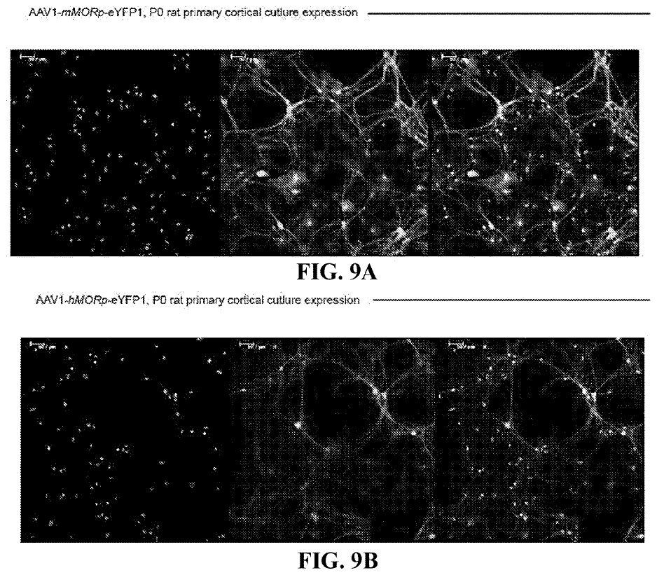

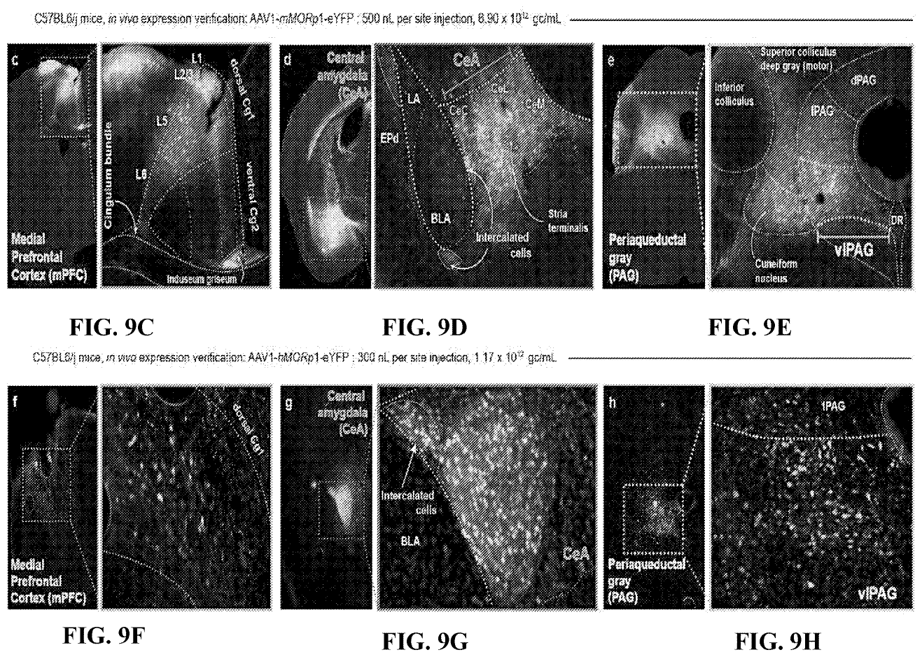

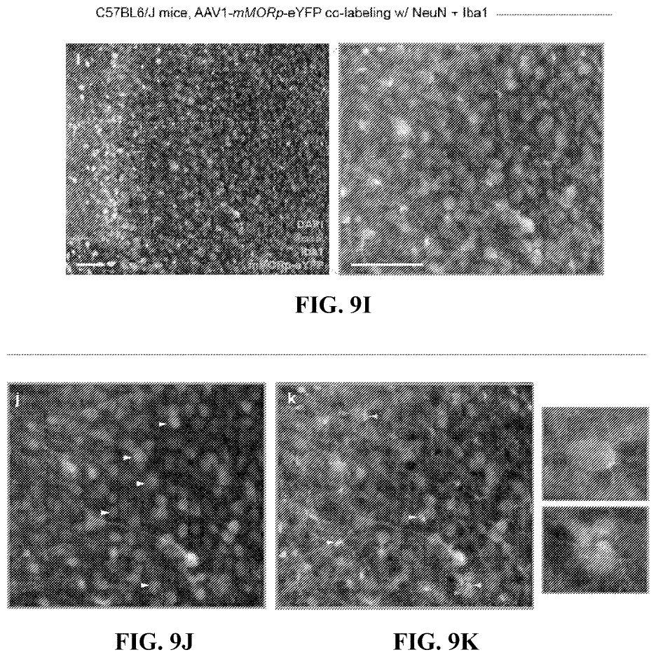

To design a new promoter system for viral-assisted, selective access to MOR+ cell-types, we analyzed a 1.9 Kb genomic region immediately upstream of the translation start codon (ATG) of the mouse mu opioid receptor gene (Oprm1) for the presence of the transcriptional initiation elements with complex transcription factor binding topology using PROMO and the Eukaryote Promoter Database (FIGS. 1A, 8A-B). Prior reports on the putative Oprm1 promoter sequence describe a “proximal” (−450 to −249 bp upstream of the ATG site) and a “distal” (−1326 to −794 bp upstream of the ATG site) region, each with transcription start sites (TSSs), which respectively account for 95% and 5% of the overall activity of the gene. Therefore, four complimentary sequences were designed that covered both the proximal and distal promoter regions within the mouse Oprm1 gene: nMORp1 (−1797 to −265, 1532 bp), mMORp2 (−1900 to −265, 1635 bp), mMORp3 (−1621 to −1, 1620 bp), and mMORp4 (−1621 to −18, 1603 bp; FIGS. 1A, 8C). In addition to these sequences targeted to the murine Oprm1 gene, we also applied our construct design protocol to generate sequences targeted to the promoter region within the human OPRM1 gene, resulting in the production of hMORp1 (−1841 to −257, 1532 bp; FIGS. 1A, 8D). All promoter sequences were assembled into plasmids encoding an enhanced yellow fluorescent protein (eYFP) reporter fluorophore and packaged into serotype 1 AAV vectors (AAV1). Administration of mMORp1-4 and hMORp1 AAVs in vitro into well plates containing primary cultured rat hippocampal neurons at concentrations of 1-3×10¹² genome copies per milliliter (gc/mL) revealed both nMORp1 and hMORp1 constructs to robustly transduce cells, as evidenced by the presence of the eYFP reporter (FIGS. 9A-9B). The four mMORp and single hMORp constructs were then tested in vivo via intracranial injection at similar concentrations into the cortex of C57BL/6J mice. The AAV1-mMORp1-eYFP and AAV1-hMORp1-eYFP constructs once again produced robust transduction and expression of reporter eYFP, while no evidence of successful transduction was noted in tissue from animals injected with mMORp2-4, possibly due to the neuron-restrictive silencer element (NRSE) and transcription factor Sp1/Sp3 binding sites near the ATG site. Further testing of mMORp1 demonstrated similar successful transduction of other brain regions in vivo, including the central nucleus of the amgydala (CeA) and periaqueductal gray (PAG) (FIGS. 1C, 9C-9G). Using mMORp1 as a template, we then designed several constructs to drive the expression of different transgenes for circuit dissection approaches within MOR+ cell-types (FIG. 1B). These variants included constructs encoding a genetically encoded calcium indicator (mMORp-GCaMP6f), a chemogenetic inhibitor (mMORp-hM4Di-mCherry), a Cre recombinase (mMORp-mCherry-IRES-Cre) and a Flp recombinase (mMORp-FlpO).

### Example 2: MMORp Viral Construct Shows Selective Expression on Oprm1/MOR Positive Neurons Across Multiple Brain Regions

Before further examining the functional applications of the constructs described above, it was first sought to confirm that the expression profile of the nMORp1 AAV was restricted predominantly to cells with Oprm1 promoter activity. Conflicting reports suggest that the MOR expression, and thus Oprm1 promoter activity, can be observed in neuronal and microglial cell types. To determine the broad cell-class that mMORp expresses within, we intracranially injected C57BL/6J mice with AAV1-mMORp1-eYFP at an optimized titer (3×10¹¹ gc/mL), targeting the mPFC as a representative, MOR+ cell containing region (N=3 male mice). The profile of transduced cells within the mPFC showed promising restriction across cortical layers 2, 5 and 6 within the cingulate (Cg1), prelimbic (PL) and infralimbic (IL) regions (FIG. 1D). These virally transduced mPFC cells were co-labeled for the neuronal marker NeuN, while none appeared to overlap with the microglial marker Iba1, despite previous reports of Oprm1 expression in microglia (FIG. 1E, 9I-9K). Apart from the underlying predilection of AAVs for infecting neurons over other glial cell types, the exclusion of expression in microglia cell-types may result from the inclusion of a PU.1 transcription factor binding region, which has been previously demonstrated to repress MOR expression in myeloid-lineage cells, including microglia.

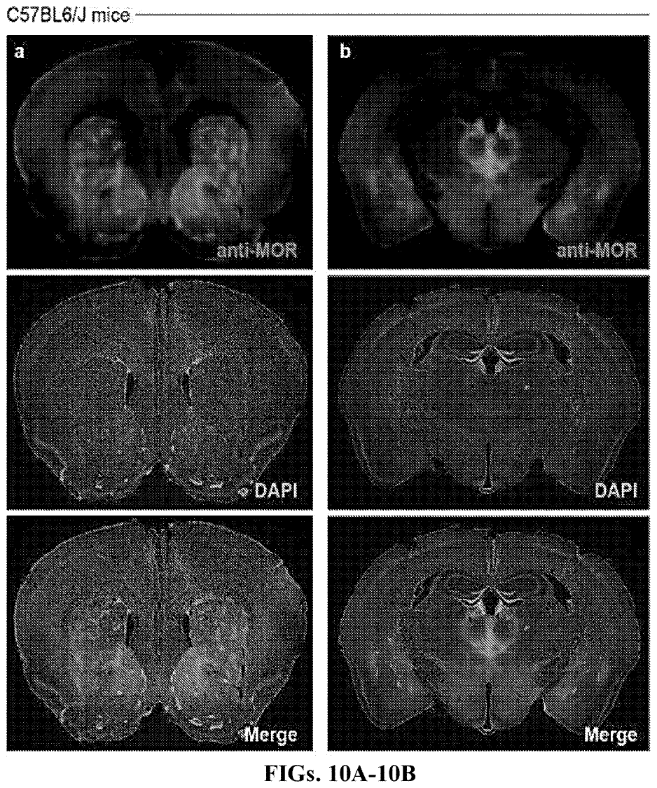

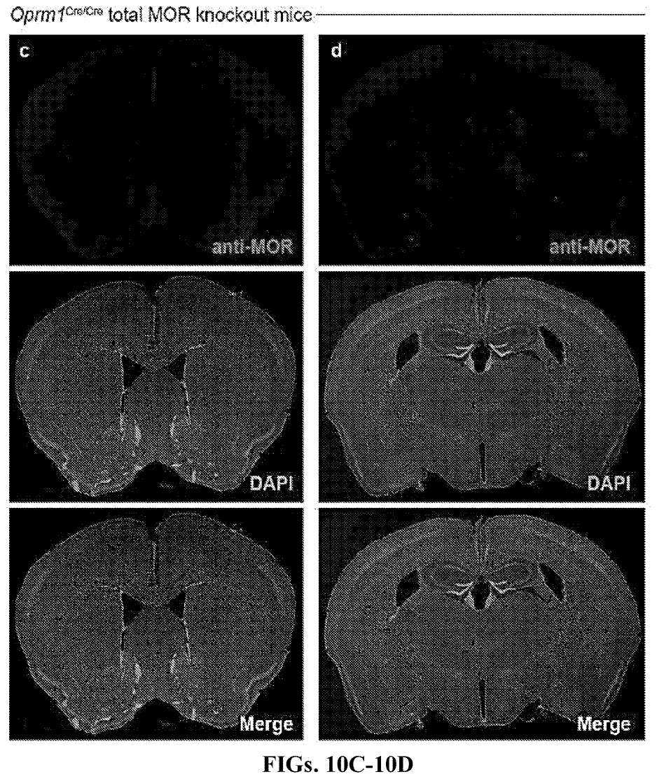

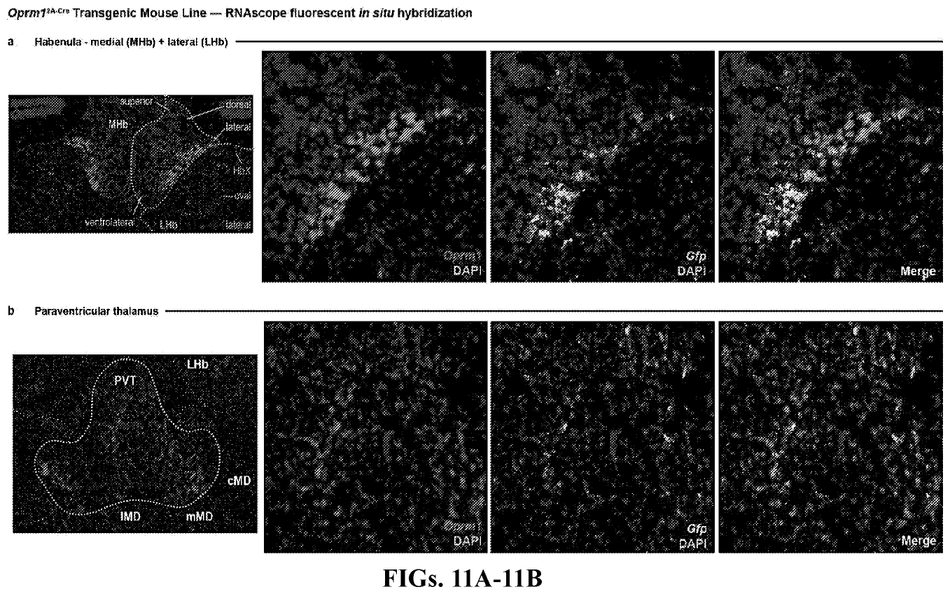

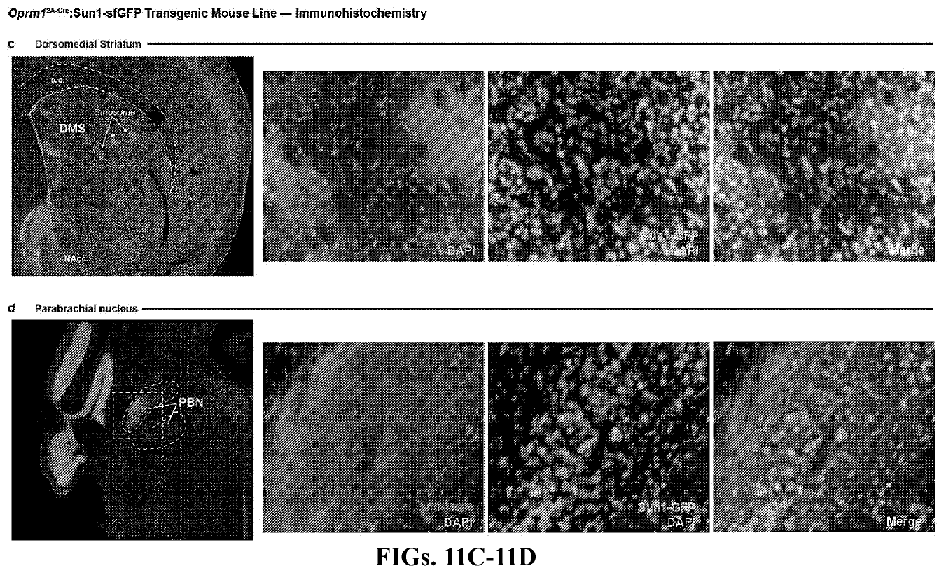

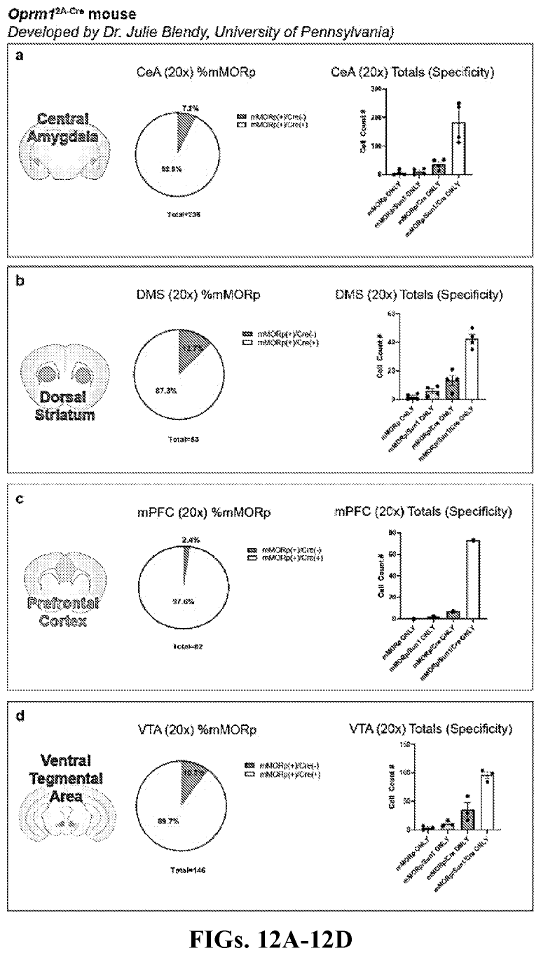

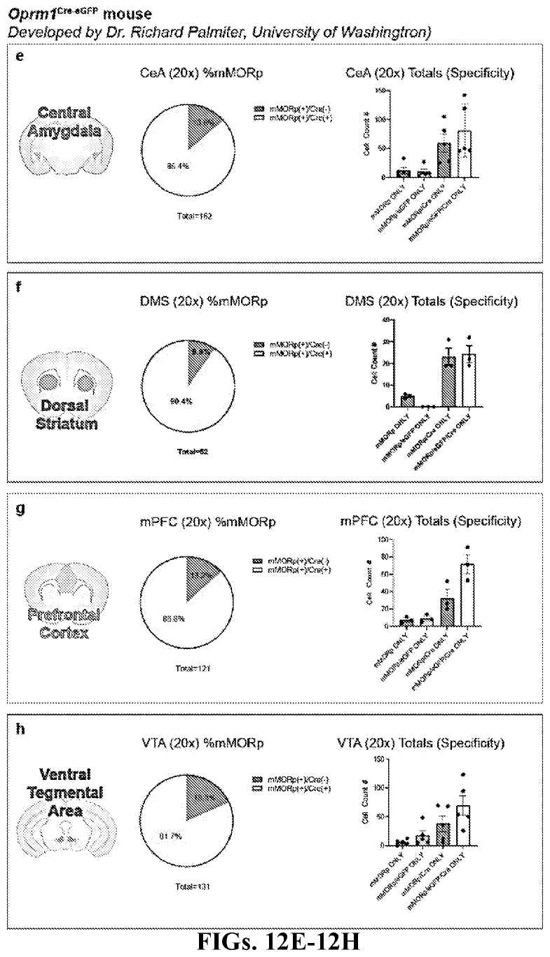

To determine the transduction efficiency and fidelity of mMORp1 to express in cells with endogenous Oprm1 promoter activities, a rigorous methodological pipeline was leveraged to assess mRNA, protein, and promoter-driven gene co-expression with mMORp encoded transgenes. First, across well-studied brain regions known to express MOR, we intracranially injected AAV1-mMORp1-eYFP at the optimized titer into the central nucleus of the amygdala (CeA), the ventral tegmental area (VTA), and the dorsomedial striatum (DMS) of C57BL6/J mice to observe viral spread and expression patterns. In the CeA, it was found that most cells showed robust co-expression of mMORp-YFP and anti-MOR immunofluorescence (via a validated MOR antibody, FIG. 10), with little expression in the immediately adjacent basolateral amygdala (BLA) and surrounding brain regions, which matches published reports of MOR expression in this broad subcortical region³⁰ (N=3 male mice, FIG. 1F). Since MOR antibody staining labels diffuse dendrites and axons, with hard-to-resolve somas, we next performed fluorescent in situ hybridization (FISH) on CeA tissue sections to determine if endogenous Oprm1 and transduced eYfp mRNA transcripts more clearly co-localized on CeA, DAPI-labeled nuclei. We observed a near total overlap of both transcript species within mMORp1 transduced cells (FIG. 1G), suggesting a high degree of specificity for our construct at the level of mRNA. However, given the dynamic and circadian expression of Oprm1, and the relatively short stability of the mRNA transcripts detectable by FISH at any one instance on a 12-μm thick section of tissue, we next used two transgenic mouse lines that express either Cre recombinase under the endogenous Oprm1 promoter, an Oprm1^{Cre} knock-in/knock-out line developed by Dr. Richard Palmiter, and a bicistronic Oprm1^{2A-Cre} line developed by Dr. Julie Blendy using CRISPR knock-in and validated by our team (see FIG. 11). Mice from the two Oprm1-Cre lines, either heterozygous for Cre (Oprm1^{Cre}, N=2 male and 3 female mice) or crossed with the nuclear-envelop targeted fluorophore Sun1-sfGFP (Oprm1^{2A-cre}:Sun1, N=2 male and 2 female mice), were injected with AAV5-mMORp1-hM4Di-mCherry at a standardized volume and titer (400 nL, 1×10¹¹ gc/mL) across four regions of interest (CeA, VTA, DMS, and mPFC), and tissue slices from successfully transduced regions were examined for mCherry co-labeling with anti-Cre immunofluorescence or Sun1-sfGFP. Sun1-sfGFP and anti-Cre staining were observed to overlap with mMORp1-mCherry signal within the CeA and all other regions of interest in Oprm1^{2A-Cre}:Sun1 injected mice (FIGS. 1H-1I), while all four transduced regions within Oprm1^{Cre} mice showed similar robust co-labeling of cells for both anti-Cre and mMORp1-mCherry signal (FIGS. 1J-1M). Analysis of the transduced regions of interests pooled from both Cre lines revealed the majority mMORp1-mCherry+ labeled neurons to be co-labeled for anti-Cre staining (CeA=90.2%%, n=9 ROIs, from N=5 mice; DMS=89.7%, n=7, N=5; mPFC=88.3%, n=4, N=3; VTA=85.2%, n=8, N=5; FIG. 1N; quantification for individual mouse lines available in FIG. 12). Minimal expression of mMORp1-mCherry signal was observed on neurons negative for anti-Cre or anti-Cre and Sun1-eGFP, further indicating that successful transduction of cells via mMORp1 (hence referred to as mMORp) is restricted predominately to putative MOR+ cells-types with active genomic Oprm1 promoters.

### Example 3: MMORp Viral Construct Displays a Robust Expression Profile in Multiple Brain Regions Across Mammalian Model Organisms

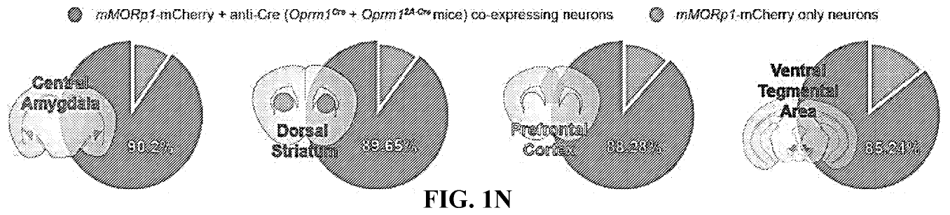

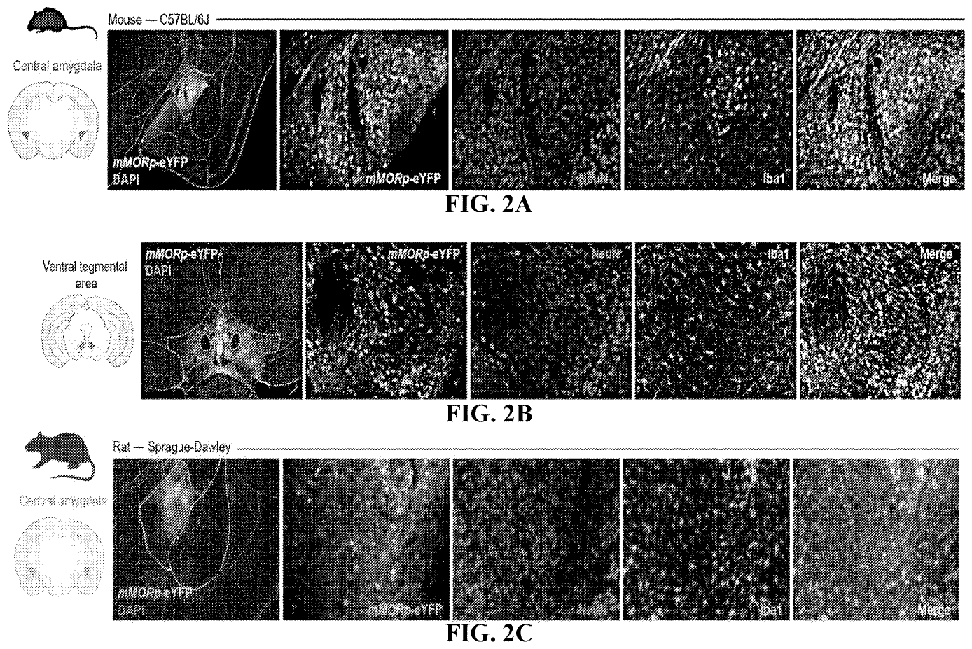

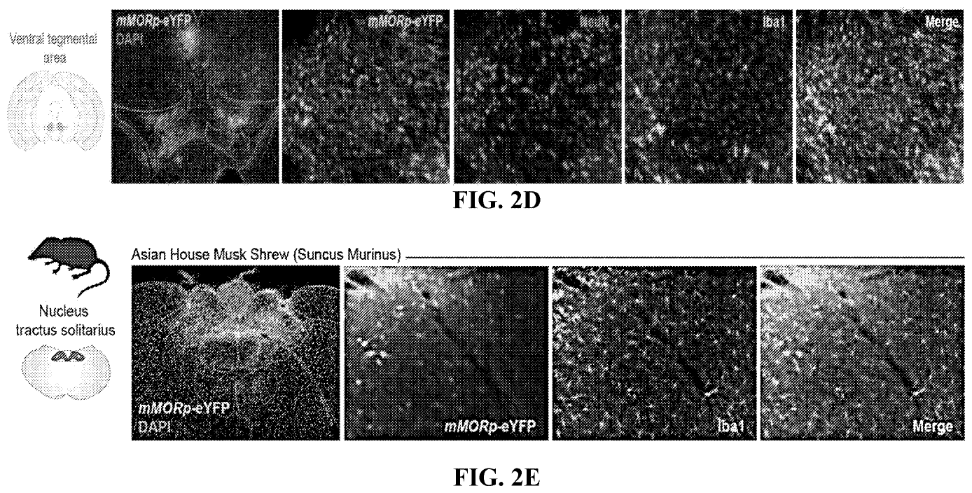

Following the specificity and selectivity studies presented previously herein, subsequence studies were undertaken in order to examine the possible applications of the viral constructs of the present disclosure in targeting populations of MOR+ neurons across several key animal model systems utilized in the broader opioid research field. While many groups utilize mice to study aspects of opioid use disorder (OUD), withdrawal, and acute/chronic pain due to a growing wealth of transgenic lines, tools, and behavioral paradigms, there remain key advantages in the use of additional mammalian model systems, in particular rats and shrews, for addressing important questions regarding the endogenous opioid system's function and dysfunction. Thus, studies evaluated the ability of mMORp to transduce cells within the brain regions of both Sprague-Dawley rats and Asian house shrews (Suncus murinus, a unique animal model to study the opioid system in the context of emesis and nausea), compared with additional C57BL/6J mice. We intracranially injected AAV1-mMORp-eYFP (1.4×1012 gc/mL) in two conserved, MOR+ representative structures in mice and rats: the CeA and VTA, and the area postrema/nucleus tractus solitarius (AP/NTS) within the dorsal vagal complex of the shrew, a structure known to express mu opioid receptors that contributes to feeding, emesis and the hypercapnic/hypoxic ventilatory response to low-oxygen. Viral expression in mouse CeA and VTA (N=3 male mice, FIGS. 2A-2B) displayed similar patterns of restriction to targeted regions of interest to what we reported above, with additional staining once again showing much of the eYFP signal to be contained within NeuN+ cell bodies and not within any cells labeled for Iba1. Tissue taken from rats targeted within the CeA and VTA (N=3 male rats, FIGS. 2C-2D) showed complementary transduction patterns to those observed in mice, with eYFP signal overlapping selectively with NeuN+ cells, and little to no overlap with Iba1+ microglia. Transfected neurons were also once again observed to be restricted primarily to the targeted regions of interest, indicating nominal levels of off-target spread or transduction of MOR− neurons. Within shrew tissue, similar patterns of transduction were also found, with mMORp-eYFP restricted exclusively to non-Iba1+ cells, and overall spread of the virus noted to be relatively restricted to the borders of the AP/NTS structure where the majority of MOR+ cells reside (N=2 female shrews, FIG. 2E). Taken together, and without wishing to be bound by theory, these cross-species studies demonstrate that the constructs of the present disclosure are highly viable in both rat and shrew models systems and show a similar pattern of restricted expression within brain structures known to harbor patches of MOR+ cells and respond to MOR manipulation.

### Example 4: MMORp-hM4Di Produces Robust Anti-Nociception in Spinal Nociceptive Circuits

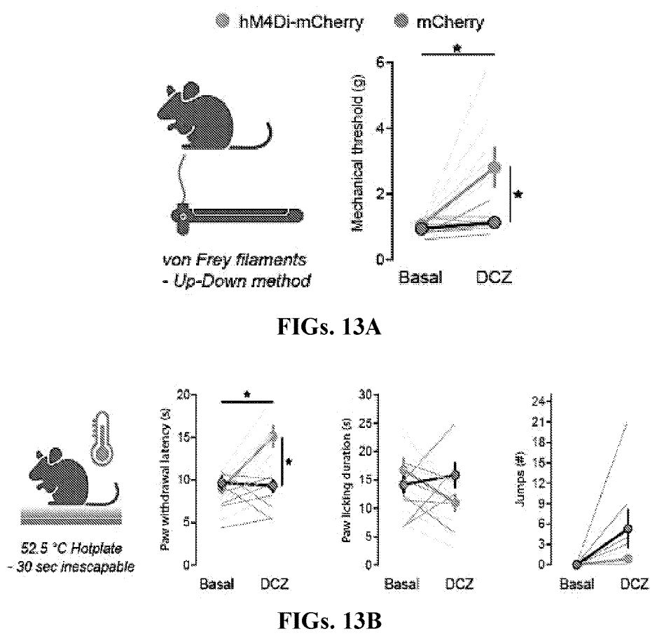

To demonstrate the application of the constructs of the present disclosure for encoding transgene products useful for interrogating opioidergic circuits in pain/nociceptive pathways, a series of functional and behavioral studies were conducted using the mMORp-h4MDi-mCherry and mMORp-GCaMP6f constructs. Intrathecal morphine and other opioids produce strong anti-nociception when binding to spinal MORs that are expressed on the presynaptic terminals of primary afferent nociceptors and on descending inputs from the brain stem, as well as MORs expressed on intrinsic dorsal horn neurons. These compounds engage inhibitory Gi signaling cascades that silence these different neural populations to disrupt the transmission of peripheral nociceptive information to supraspinal brain regions. However, behavioral pharmacology does not allow for testing the role of different MOR+ neural populations within this critical nexus of the nociceptive pathways regarding analgesia. Since the DREADD GPCR, hM4Di, couples to similar Gi cascades as MOR, we hypothesized that mMORp-hM4Di could mimic intrathecal morphine antinociception, but in a selective manner that tests the necessity of spinal cord MOR+ neurons to induce analgesia, independent of afferent and brainstem terminal opioid-mediated inhibition. Thus, for chemogenetic manipulation of spinal MOR+ neurons in behaving mice experiencing nociceptive stimuli, we injected C57BL/6J mice at the L4-L5 lumbar spinal cord with AAV1-mMORp-h4MDi-mCherry (intraparenchymal, 400 nL, ˜3×10¹² gc/mL, per injection site; FIG. 3A). Robust mMORp-hM4Di-mCherry expression was detected throughout the dorsal and ventral horns, primarily in lamina II inner, consistent with expression patterns observed in MOR-mCherry mice, without detection in dorsal root ganglia axonal inputs (N=3 male mice; FIGS. 3B, 3C). A series of well-validated, common nociceptive behavioral assays were then conducted on these mice with and without different DREADD agonists to measure anti-nociceptive effects (FIG. 3D). Results demonstrated that mMORp-hM4Di injected animals displayed an increase in mechanical threshold sensitivity in the von Frey filament Up-Down test following either intrathecal deschloroclozapine (DCZ, 10 pg; two-way ANOVA, F=8.911, P=0.0105; FIG. 13A) or systemic clozapine N-oxide (CNO, i.p., 3 mg/kg; two-way ANOVA, F=8.521, P=0.0112) compared to the within-subjects baseline thresholds and mCherry control mice (FIG. 3E). Similarly, the mMORp-hM4Di group showed an increase in reflexive latency to withdrawal, and decreases in the duration of paw licking behavior and jumping or escape-related events on an inescapable hot plate (52.5° C.) compared to control animals following either DCZ (hot plate latency: two-way ANOVA, F=19.81, P=0.0007, duration: F=2.058, P=0.1751, jump/escape: F=4.921, P=0.0450; FIG. 13B), or CNO treatment (duration: two-way ANOVA, F=9.514, P=0.0081; jump/escape: 2-way ANOVA, F=13.65, P=0.0024; FIG. 3F). Lastly, mMORp-hM4Di and mCherry groups received systemic CNO 30 min prior to an intraplantar injection of a 4% formalin solution to induce TRPA1-nociceptor hypersensitivity and subsequent central sensitization. In the mMORp-hM4Di mice we observed significant decreases in nocifensive behaviors (paw licking, biting, escape behaviors, etc.) during both the first stage of behavioral observations (direct activation of afferent nociceptors) and the later second stage (reflective of the inflammatory and central sensitization component) of the test (FIG. 3G; time: two-way ANOVA, F=22.27, P<0.0001; phase: two-way ANOVA, F=401.5, P<0.0001). In total, these tests demonstrate the functional viability of our chemogenetics-based viral constructs in behaviorally relevant tasks, as exemplified by the ability of mMORp-hM4Di to produce similar antinociceptive effects to those of spinal opioid agonist administration.

### Example 5: MMORp-GCaMP6f Permits Readout of Neuronal Population Activities in Response to Morphine, Opioid Withdrawal, and Noxious Stimuli

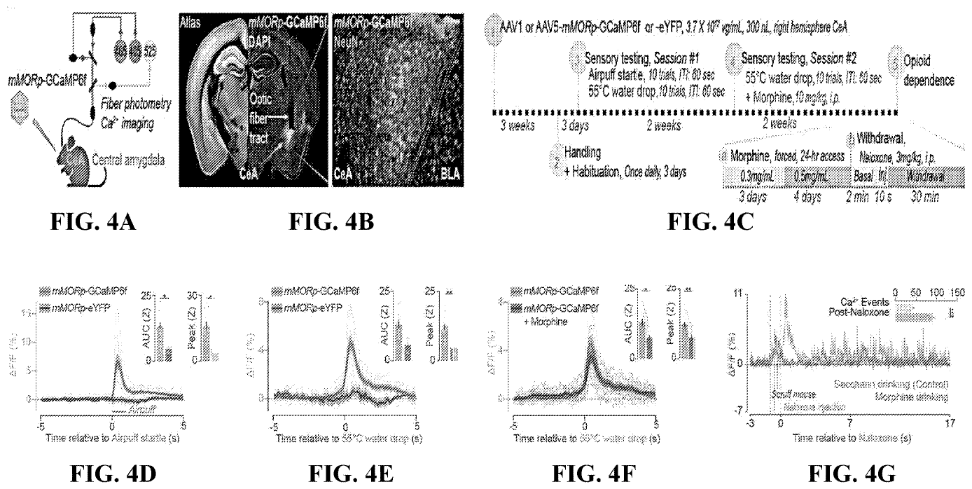

Studies next assessed the ability of the mMORp-GCaMP6f construct for use in fiber photometry calcium imaging in awake, behaving animals to assess population activity changes of transduced MOR+ CeA neurons, a sub-region of the amygdala shown previously to express MORs and to be implicated in aspects of nociceptive processing. C57BL/6J mice were injected with AAV1-mMORp-GCaMP6f (400 nL, ˜2×10¹² gc/mL; N=12 male mice) or an eGFP encoding control virus (N=3 male mice) in the right CeA (FIG. 4A) prior to implantation of a fiber optic head post just dorsal to the injection site. As with the other mMORp constructs, mMORp-GCaMP6f expressed strongly, with relatively restricted expression in the CeA and minimal spread into the neighboring BLA or striatum (FIG. 4B). Following three weeks incubation period, mice were run through a battery of behavioral experiments to gauge relative calcium activity changes in response to noxious stimuli (FIG. 4C). Initially, mice were exposed to brief air puffs delivered to the abdomen (compressed air canister, 1 sec puff) and hot water drops applied to the left hind paw (˜55° C., ˜25 μl drop), and the recorded calcium mediated events were time-locked to the stimulus application. In response to air puffs, calcium activity in the CeA of mMORp-GCaMP6f injected mice increased significantly relative to eGFP control animals when examining both the area under the curve (AUC) and peak z-score of recorded events (AUC: GCaMP=13.2, eGFP=4.7, two tailed unpaired t-test, P=0.0351; peak z-score: GCaMP=15.9, eGFP=3.7, two tailed unpaired t-test, P=0.0112; FIG. 4D), thereby indicating proper functionality of our GCaMP6f construct in CeA neurons, a region known for its elevated activity profile in response to unexpected external stimuli. Similarly, hot water applications reliably produced robust, time-locked calcium mediated events when compared with controls (AUC: GCaMP=13.0, eGFP=5.7, two tailed unpaired t-test, P=0.051; peak Z-score: GCaMP=12.3, eGFP=4.5, two tailed unpaired t-test, P=0.009; FIG. 4E). To assess if mMORp-GCaMP6f-transduced CeA neurons are opioid-sensitive, morphine (i.p., 10 mg/kg) was then administered 30 minutes prior to another round of hot water hind paw stimulations. Morphine treated mMORp-GCaMP6f animals displayed a marked reduction in calcium mediated event AUC and peak amplitude (AUC: no morphine=13.0, morphine=7.7, two tailed unpaired t-test, P=0.016; peak z-score: no morphine=12.3, morphine=7.6, two tailed unpaired t-test, P=0.005; FIG. 4F), indicative of a potential effect of morphine on the activity of transduced, putative MOR+ CeA neurons.

Subsequent studies next sought to extend the utility of our mMORp-GCaMP6f construct to examining applications aimed at modeling chronic opioid use and withdrawal. Using the same cohort of mMORp-GCaMP6f mice, we placed animals on a forced morphine drinking paradigm using home cage water bottles containing either an increasing concentration of morphine, or the artificial sweetener saccharin (N=5 male mice, saccharin; N=6 male mice, morphine). Drug treated mMORp-GCaMP6f groups were supplied with water containing 0.3 mg/ml morphine, 0.2% saccharin for 3 days, and 0.5 mg/ml morphine, 0.2% saccharin for 4 days, while control mMORp-GCaMP6f groups were only provided 0.2% saccharin laced water (FIG. 4C). After the final day of drinking, mice underwent a precipitated withdrawal challenge (naloxone, i.p., 3 mg/kg) prior to the start of behavioral testing and fiber photometry recording. Morphine drinking, naloxone treated mMORp-GCaMP6f animals displayed characteristic opioid withdrawal behavioral phenotypes (wet dog shakes, biting, jumping/escape behavior) immediately following naloxone injection, with an associated, significant increase in the total number of calcium-mediated events scored within a 20-minute observation window. By comparison, saccharin drinking nMORp-GCaMP6f animals showed little to no changes in overt behavior and calcium-mediated activity profiles for the duration of testing (average post naloxone calcium events: saccharin=41, morphine=91, two tailed unpaired t-test, P=0.0007; FIG. 4G). Taken together, and without wishing to be bound by theory, these use case experiments demonstrate that our mMORp-GCaMP6f construct can be paired with widely adopted techniques for recording in vivo neural activities in pain and OUD-related studies.

### Example 6: MMORp Driven Recombinases and PHP.eB/PHP.s Capsid Packaging Allows for Intersectional Genetic Access to PNS and CNS Opioidergic Cell Populations and Circuits

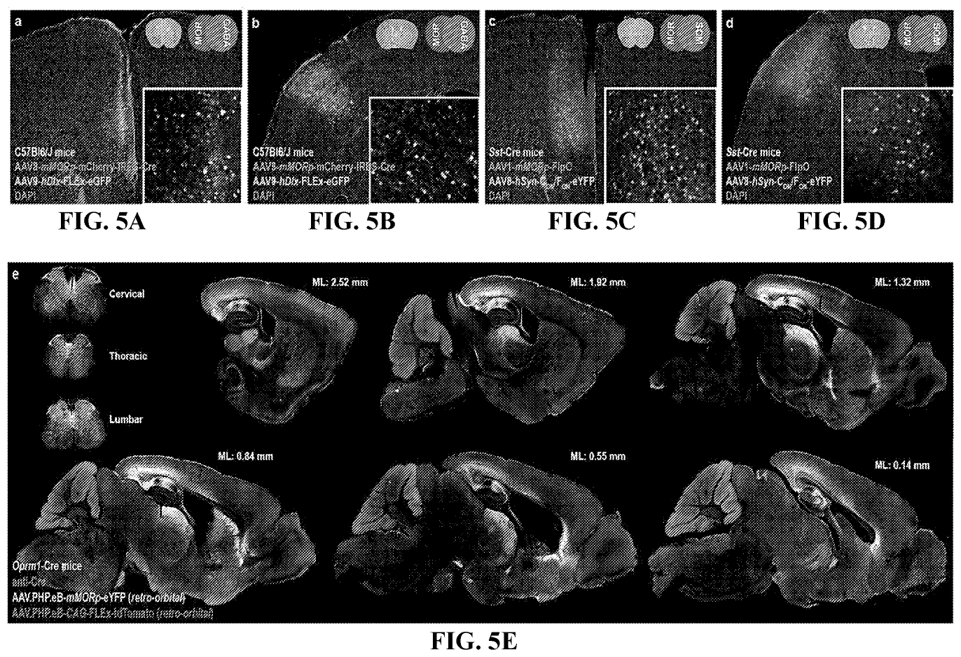

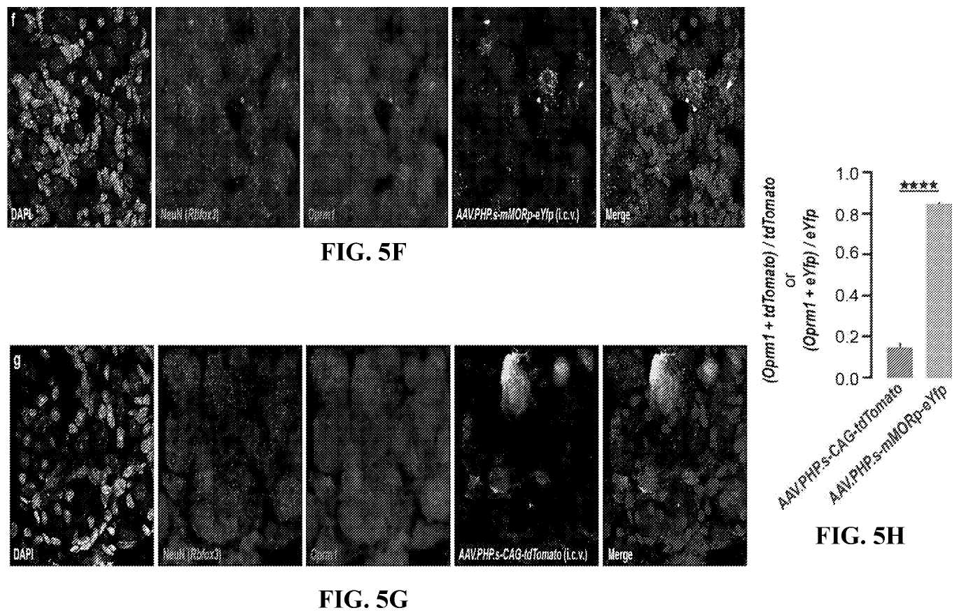

Increasingly, many contemporary neuroscience investigations aim to both manipulate specific neuronal subpopulations and circuits based on multiple dimensions, including molecular expression, connectivity, function, and location within the nervous system, with viral tools becoming an increasingly invaluable means to achieve these levels of specificity. Determining which cell-types are accessed can be partly controlled by the capsid proteins of a specific virus, such as the recently engineered CNS transducing PHP.eB and the PNS transducing PHP.S capsids, as well as the inclusion of specific promoter and/or enhancer element into constructs packaged within AAVs. Furthermore, hundreds of existing Cre recombinase transgenic mouse lines are also in use in labs around the world which can be used in combination with other virally encoded recombinases such as Flp to achieve cell and circuit specific genetic access. To capitalize on these advances in targeting strategies and tool development, four example mMORp constructs were created that are useful for intersectional neuronal labeling and tracing studies: a mMORp-mCherry-IRES-Cre construct encoding Cre recombinase, a mMORp-FlpO construct encoding Flipase, and two mMORp-eYFP constructs packaged in AAV-PHP.eB and AAV-PHP.s capsids. Single-cell RNA sequencing studies show that Oprm1 is expressed in both glutamatergic pyramidal cell-types as well as GABAergic interneurons. Selective access to these different classes of cortical neurons however has not been possible, leaving gaps in our basic understanding of cortical opioidergic processes. Thus, to test our mMORp-driven recombinases for their effectiveness in targeting GABAergic subtypes of MOR+ cortical neurons, we first co-injected a mixture of AAV8-mMORp-mCherry-IRES-Cre (titer: 7×10¹² gc/mL) combined with AAV9-hDlx-FLEx-eGFP (titer: 2.2×10¹³ vg/mL) viruses into the mPFC and somatosensory cortex (S1) of C57BL/6J mice (N=3 male mice). The latter construct uses the human D1x enhancer element to promote viral transduction in forebrain GABAergic cells and contains a FLEx switch making eGFP transgene expression Cre-dependent. The same expression pattern of mCherry in mPFC was observed across the cortical layers as shown in FIG. 1D for our first round of mMORp-eYFP injections, which was mirrored in S1 brain sections, while eGFP expression was restricted to a subset of small cells with non-pyramidal morphology in both mPFC and S1 (FIGS. 5A-5B). Next, using a somatostatin (SOM) Cre transgenic mouse line (Sst-Cre, N=3 male mice), AAV1-mMORp-FlpO (titer: 1.4×10¹² gc/mL) was coinjected with virus comprising a pan-neuronal, human Synapsin promoter-driven Cre- and Flp-dependent eYFP reporter (AAV8-hSyn-CON/FON-eYFP, titer: 2.4×10¹³ vg/mL) to implement an INTRSECT (INTronic Recombinase Sites Enabling Combinatorial Targeting)-based targeting strategy to label putative SOM+/MOR+ neurons. Results found that mMORp-FlpO successfully integrated with the INTRSECT system and observed numerous eYFP labeled cells in both mPFC and S1 (FIGS. 5C-5D). Taken together, these two intersectional strategies show that the mMORp constructs of the present disclosure can also be combined with the ever-expanding catalog of recombinase transgenic animals and INTRSECT viral toolsets to access a multitude of cell-types in the brain.

Next, studies evaluated the use of the PHP.eB and PHP.s capids to deliver mMORp-eYFP into broad, CNS and PNS distributed MOR+ cells. In adult Oprm1^{Cre} transgenic mice, a mixture of AAV-PHP.eB-mMORp-eYFP (titer: 8.6×10¹² gc/mL) and AAV-PHP.eB-CAG-FLEx-tdTomato (titer: 2.1×10¹³ vg/mL) was delivered via retro-orbital injection (50 μl, 50:50 mix), and found co-expression of eYFP and tdTomato in anti-Cre+ cells throughout the spinal cord and brain (FIG. 5E). In C57BL/6J P2 pups, we performed an intracerebroventricular injection of AAV-PHP.S-mMORp-eYFP (1.4×10¹³ gc/mL, FIG. 5F) and after 6 weeks incubation, performed FISH on dorsal root ganglia sections for eYfp and Oprm1 mRNA transcripts. When compared to pups that had been similarly injected with a generic AAV-PHP.S-CAG-tdTomato encoding virus (titer: 2.1×10¹³ vg/mL, FIG. 5G), cells counted in DRG sections from the mMORp injected animals (n=4 ROIs) showed robust co-localization of eYfp and Oprn1 transcripts on/around the same DAPI delineated nuclei, while co-localization of tdTomato and Oprm1 transcripts (n=6 ROIs) was significantly sparser (Oprm1+tdTomoto/tdTomato=0.15, Oprm1+eYfp/eYfp=0.85, two tailed unpaired t-test, P=0.0011, FIG. 5H). Taken together, and without wishing to be bound by theory, these representative findings highlight the use of our mMORp constructs in combination with viral capsid engineering to selectively target MOR+ primary afferents and large numbers of spinal and brain neurons.

### Example 7: Human and Mouse MORps Provide Unique Access to Opioidergic Cells in Non-Human Primates In Vivo, and Human-Derived Neuronal Cell Cultures In Vitro

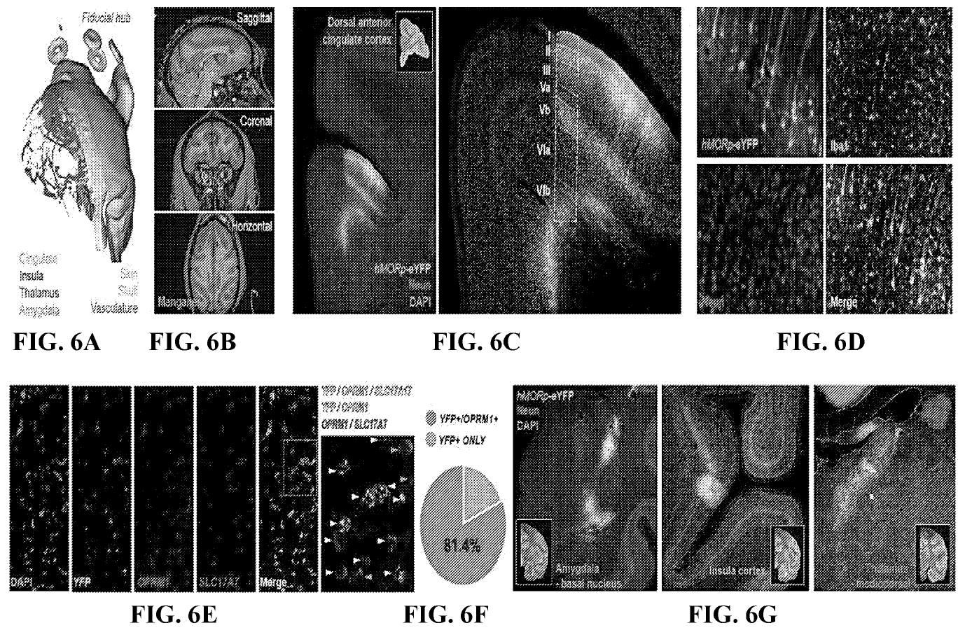

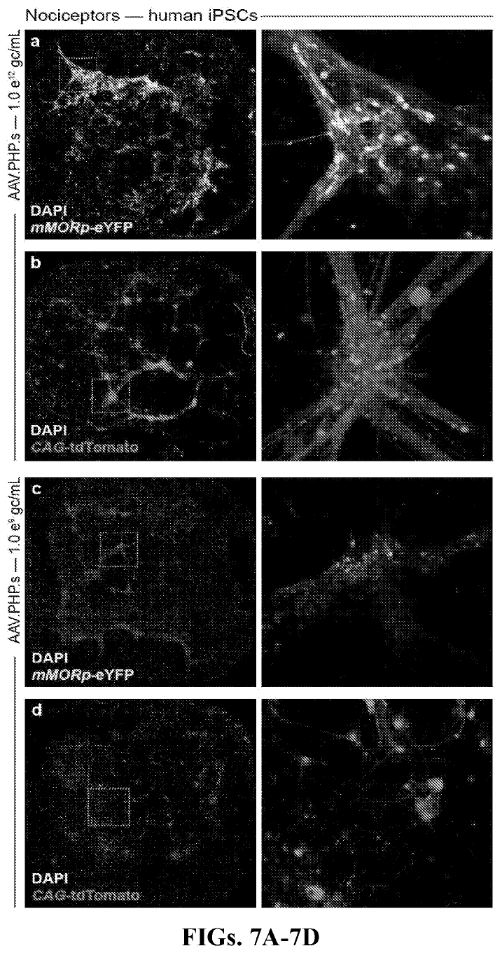

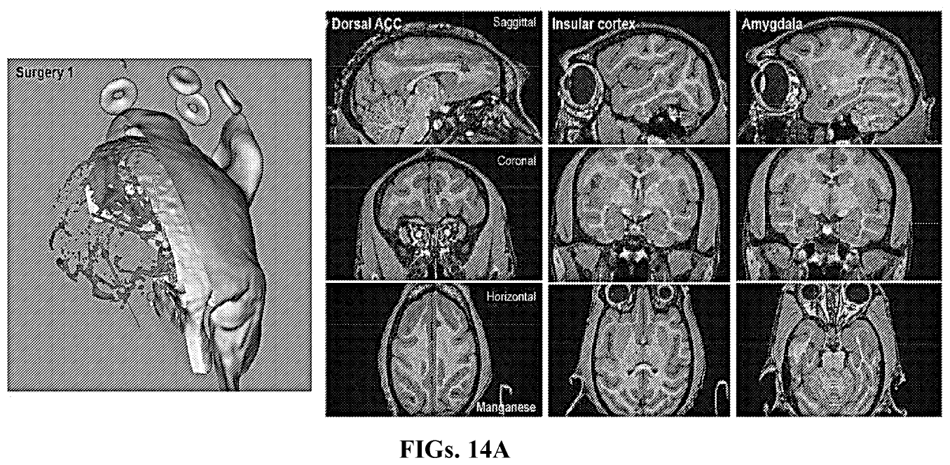

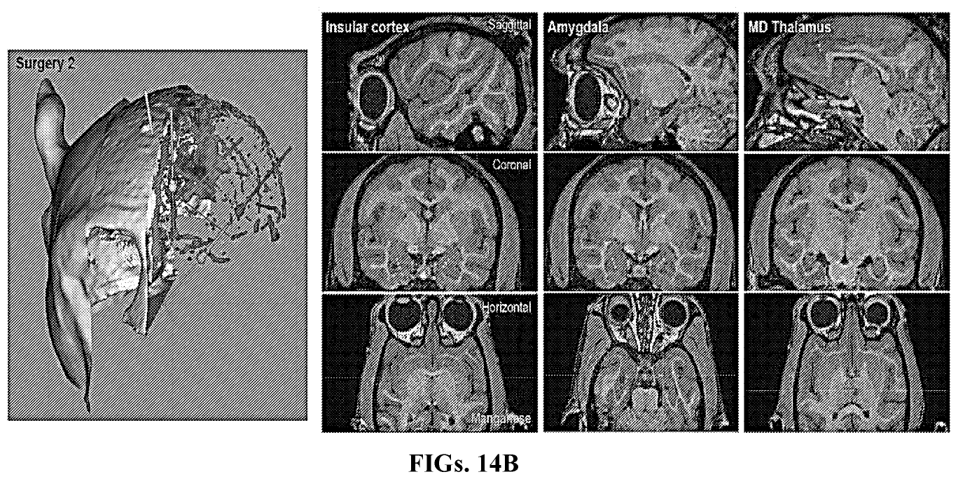

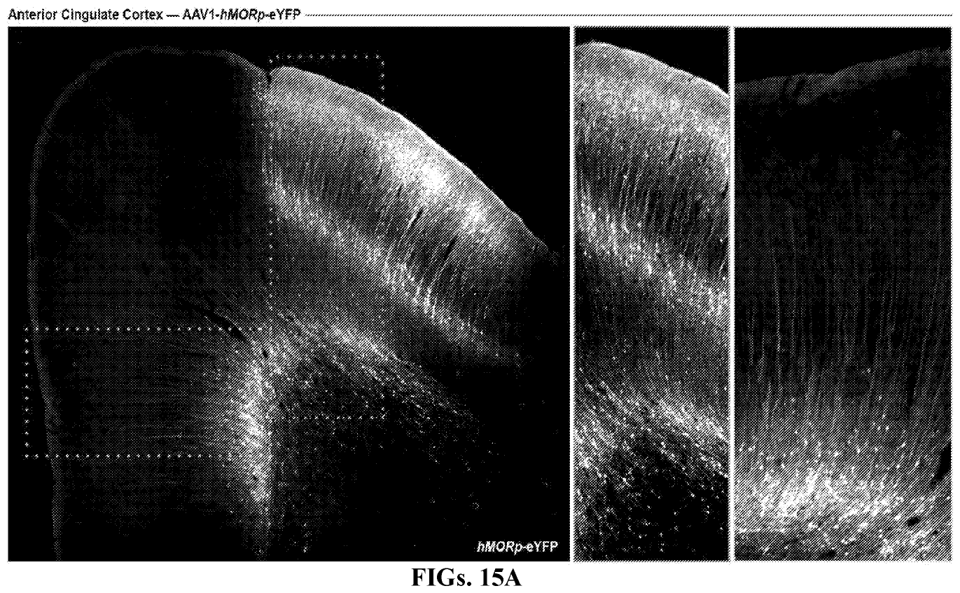

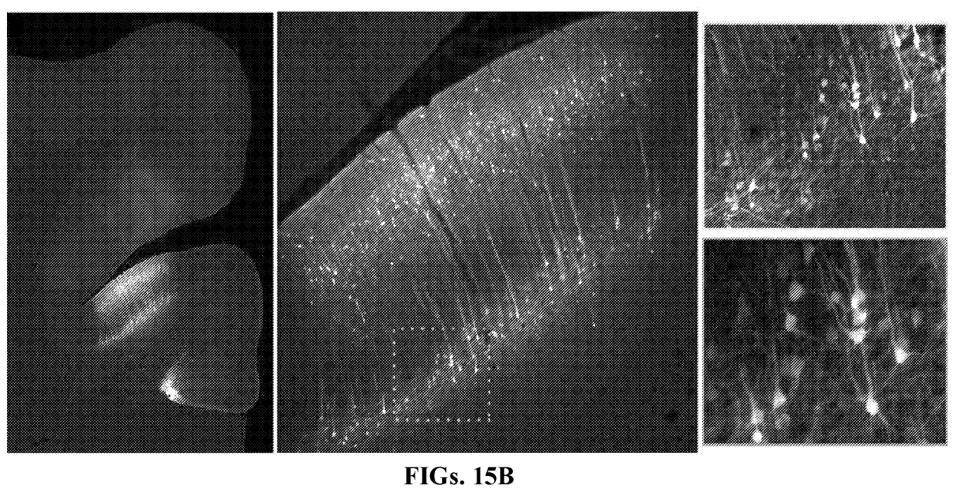

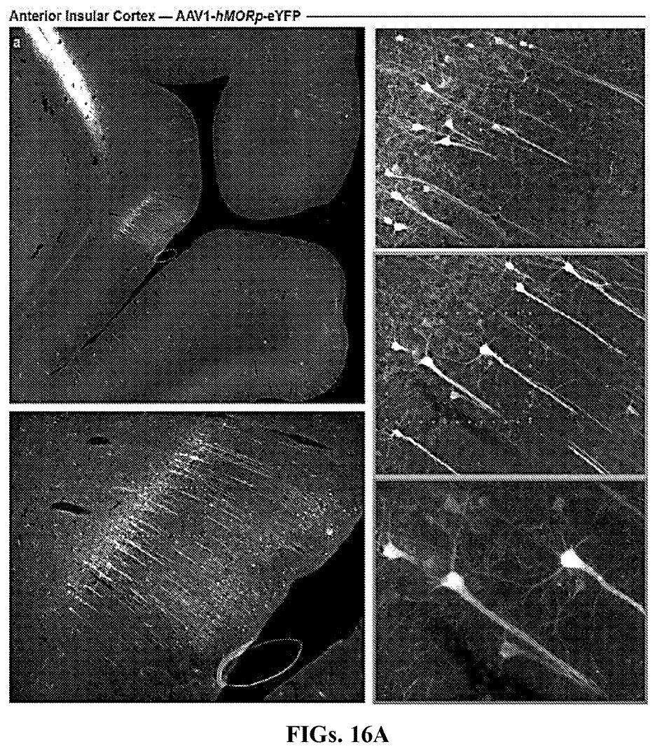

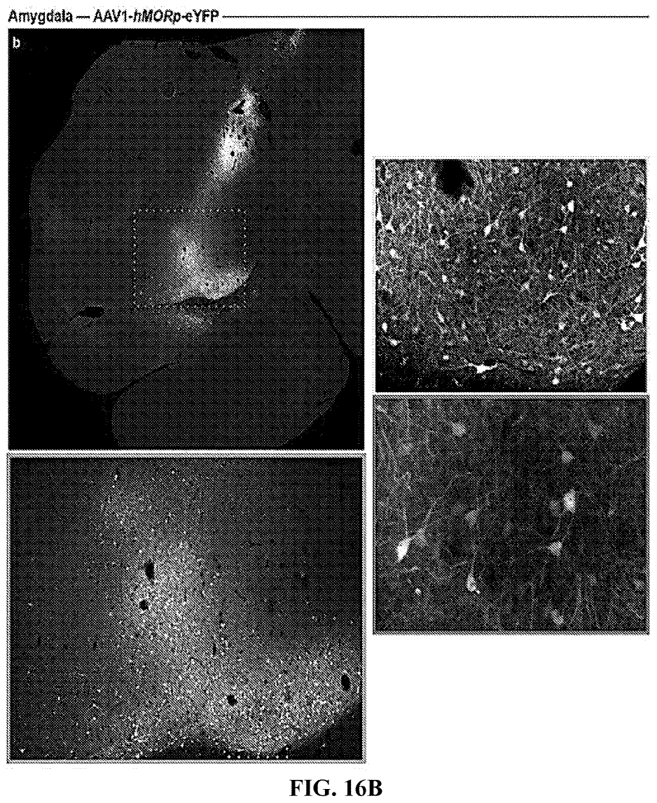

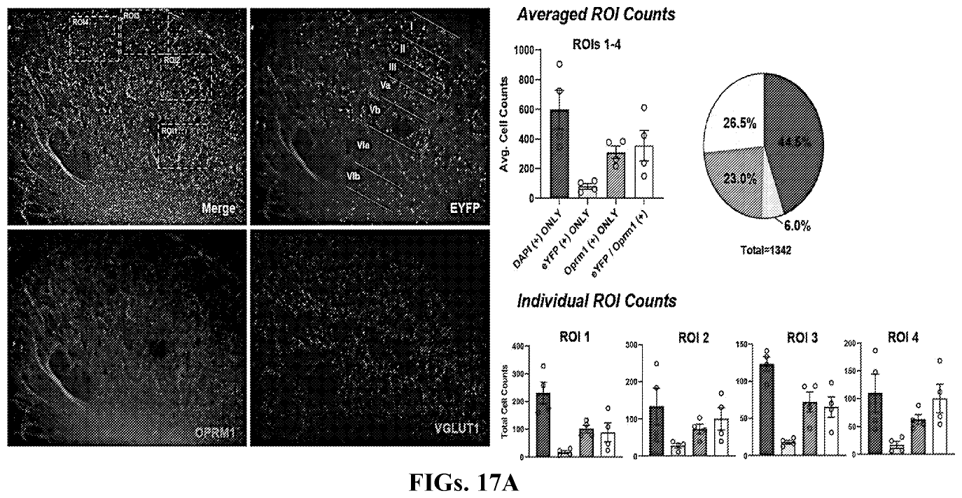

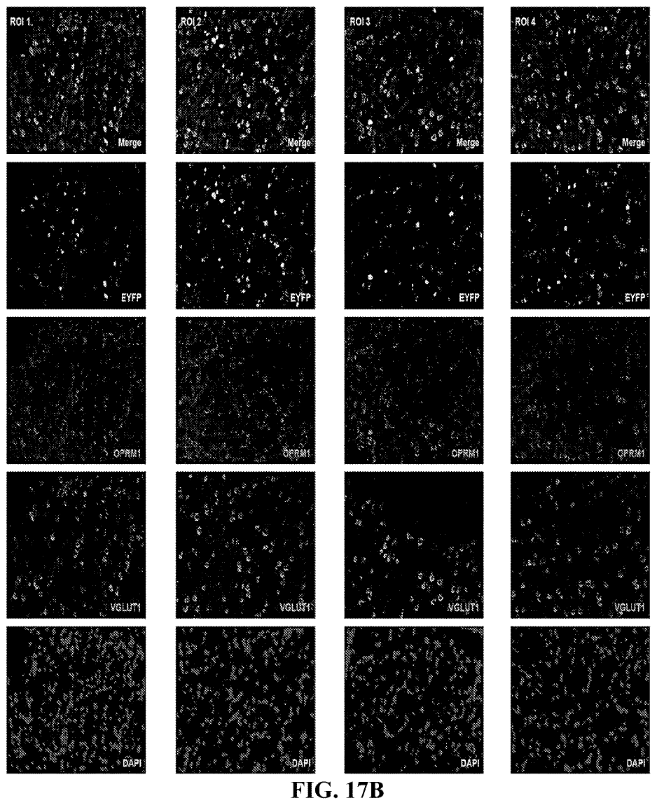

Having characterized our mMORp constructs in rodents and other small mammalian model systems, we next sought to determine the viability of our human promoter derived MORp construct (hMORp). We first performed a series of intracranial injections using the AAV1-hMORp-eYFP virus (titer: 1.7×10¹² gc/mL) in a single male rhesus macaque, targeting two regions anatomically complementary to those we had examined for transduction efficiency of the mMORp constructs in our rodent studies, the dorsal anterior cingulate cortex (dACC) and the amygdala, as well as the insular cortex and medial thalamus (FIGS. 6A-6B, FIG. 14). Tissue sections taken from dACC revealed robust transduction of NeuN+ neurons throughout multiple layers of the cortex, with the greatest densities of eYFP+ neuronal cell bodies localized to cortical layers II, V and VI, and the processes of these neurons noted to extend into/throughout layers 1, III, V and VI (FIG. 6C, FIG. 15). Immunostaining in the dACC for microglia with anti-Iba1 showed no overlap with eYFP+ cells, suggesting that like the nMORp constructs, the hMORp construct preferentially targets putative MOR+ neurons and not glial cells (FIG. 6D). To confirm hMORp selectivity in MOR+ cell-types, FISH was performed on dACC sections and quantified the co-localization of transgene EYFP mRNA transcripts with macaque OPRM1 mRNA transcripts across four regions of interest (ROIs). We noted that the majority of OPRM1 labeled cells throughout the transduced region of the dACC were co-labeled for EYFP transcript compared to those cells labeled for OPRM1 or EYFP alone (OPRM1/EYFP+=˜81.4%, EYFP+/OPRM1−=˜18.6%; FIGS. 6E-6F; expanded quantification and individual ROI counts presented in FIG. 17). Successful transduction of neurons expressing the OPRM1 promoter was also observed in the amygdaloid structure, insula and mediodorsal thalamus (FIG. 6G), with tissue samples containing the insular cortex and/or the amygdala also showing robust expression of the eYFP tag within cell bodies and processes in both regions (FIG. 16), suggesting a broad application for hMORp to provide genetic access to MOR+ cells in multiple regions of the brain in non-human primate, genetically intractable subjects.

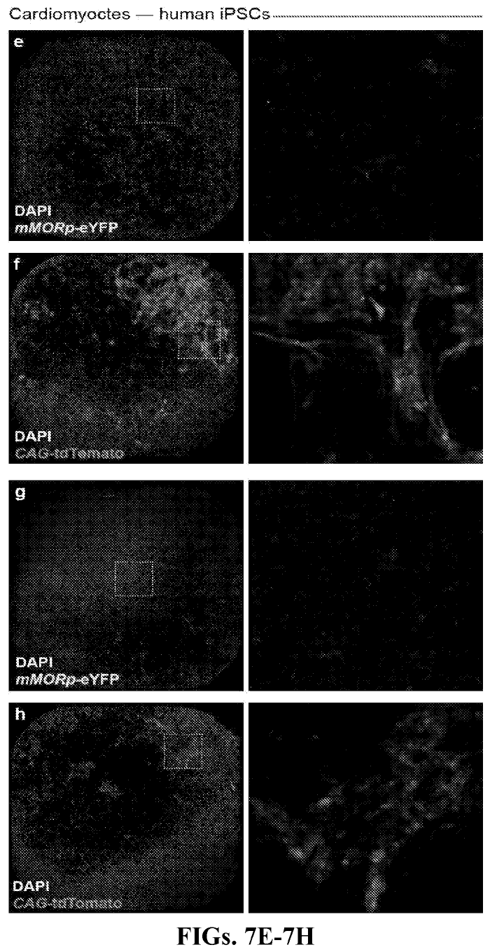

Lastly, several studies were conducted to determine whether the MORp viruses disclosed herein would be able to provide genetic access to these same putative MOR+ cells within human derived model systems. To test this, human induced pluripotent stem cells (iPSCs) were cultured and differentiated to produce either nociceptor-like neuronal cells or non-neuronal cardiomyocytes, both of which were then treated with direct administrations of either the AAV-PHP.S-mMORp-eYFP of the present disclosure or a control CAG-tdToniato virus at four different titers (1×10⁹-10¹² gc/mL). Broad transduction of cells was observed with both viruses in nociceptor cultures that increased with titer concentration, with robust expression of both reporters noted at the 1×10¹² gc/mL titer most prominently (FIGS. 7A-7D). By contrast, while expression of tdTomato signal remained prominent at both low and high viral titer concentrations in cultured cardiomyocytes, no eYFP signal was noted within these cultures following AAV-PHP.S-mMORp-eYFP treatment (FIGS. 7E-7H), indicating that our current iteration of MORp constructs appear to show selectivity for human cell types known to express MOR when compared to cells shown to possess low expression of this receptor on them from previous studies.

### Example 8: DORp and KORp Viral Construct Shows Selective Expression in Oprd1+ and Oprk1+ Neurons

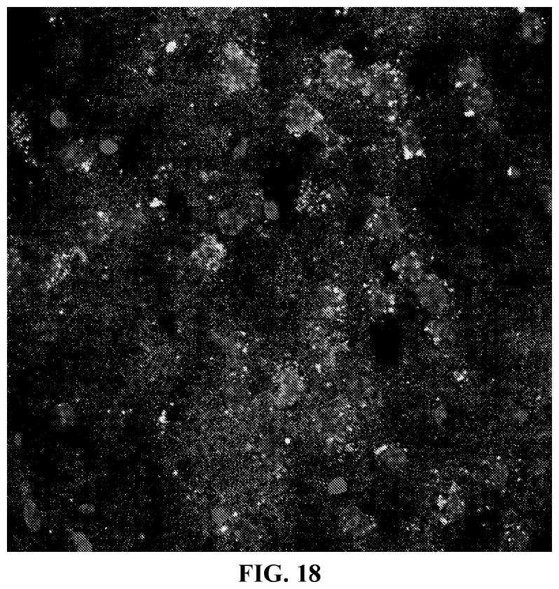

A series of constructs were then assembled which comprised promoter sequences derived from the delta opioid (DORp) and kappa opioid (KORp) receptor genes (Oprd1 and Oprk1, respectively). A kappa opioid receptor promoter/YFP construct (KORp-eYFP) was then packaged into an AAV5-based viral vector (AAV5-KORp-eYFP), which was then used to transduce Oprk1+ neuronal cells. Following transduction, the cells were then assessed for YFP expression. FIG. 18 illustrates the robust selectivity of the KORp promoter to drive transgene expression in endogenous KOR-expressing brain neurons.

### Example 8: Selected Discussion

The use of up and downstream genetic elements to target virally deliverable transgenes to select neural structures, subtypes of neuronal populations and circuits within them represents a burgeoning area of research within the gene delivery field. The results disclosed herein demonstrate that these principles can be extended to targeting mu opioidergic neuronal populations throughout the CNS and PNS with a high level of specificity and selectivity, and that the study of these transduced cells via effector transgenes yields behavioral and physiological readouts consistent with MOR manipulation. These findings present broad implications for the use of these viral tools in the study of the neurobiology of opioid-related fields and open the door to the translational use of such tools for potential therapeutic development and screening platforms.

The versatility of both the mMORp and hMORp constructs to transduce putative MOR+ cells in multiple conserved brain structures long shown to harbor mu opioidergic cells across multiple species represents an important step towards expanding the use of different model systems in opioid circuit neuroscience investigations. While five Oprm1-Cre transgenic mouse lines have been created, at present only one is commercially available (Jackson Labs, Strain:03557). These mice are haplo-insufficient or total knockouts for Oprm1 when hetero- or homozygous for the Cre allele, respectively, which needs to be accounted for when designing experiments to investigate MOR function itself. The use of AAVs and our MORp constructs can provide a more cost-effective and rapid method for labs to dissect the mu-opioid receptor system in vivo than expensive and slow rodent breeding schemes. Importantly, there are no other transgenic species models available that provide genetic access to MOR cell-types. The fact that non-human primate and other small mammalian systems lack the tools or transgenics to achieve the same level of granularity in the study of opioid neurobiology has placed an overreliance on mouse systems that may not be the most appropriate model of studies for areas such as pain perception, opioid addiction-like behaviors, or cognitive processes involving the endogenous opioid system. The success of our constructs to transduce MOR+ cells in vivo in mouse, rat, shrew and macaque model systems demonstrate them to be potentially useful for researchers working across species that wish to target these populations for anatomical and functional studies.

Regarding the functional identify of the cells transduced with these viruses, and the utility of the effector transgenes encoded by them, the mouse validation studies disclosed herein suggest these cells to respond to both behavioral and pharmacological manipulation in a manner consistent with MOR agonism and/or antagonism. Within the spinal cord, the activation of mu receptors on cells in the DRGs has previously been demonstrated to produce a robust decrease in overall cellular activity and excitability, consistent with MOR's function as an inhibitory G protein coupled receptor, as well as a reduction in nocifensive behaviors following intrathecal administration of MOR agonists. As disclosed herein, chemogenetic studies using an hM4Di encoding mMORp construct produced similar effects, consistent with a more selective modulation of MOR and putative MOR+ cells within the region. Noted increases in the number of calcium mediated events in the CeA of mice injected with the mMORp-GCaMP construct were also consistent with previous studies demonstrating pain-responsive CeA neurons to increase their excitatory activity in response to noxious stimuli, and the CeA in general to show an overall increase in molecular markers of neuronal activity in paradigms examining the effects of naloxone precipitated opioid withdrawal. It is thus likely that our viral constructs are indeed transducing the desired target cell populations within these regions, as our IHC and ISH results additionally support. The results of these experimental examples highlight the power and versatility of the MORp tools of the present disclosure in the study of the opioidergic system not just regarding the modulation of physiological pain responses, but also in the study of chronic opioid addiction, withdrawal and other emerging complex behavioral features of pain and OUD.

While most of the work presented herein delves into the applications of the mMORp constructs and their basic research utility in small animal models, the success of the hMORp and mMORp constructs to transduce MOR/OPRM1+ cells in rhesus macaque in vivo, as well as differentiated human nociceptor cultures in vitro, respectively, suggests greater applications for these tools in translationally minded research. Apart from opening a door into the study of pain responsive neuronal populations in higher order animal models, the ability to directly target and manipulate opioidergic cells in human culture samples could greatly improve the specificity of drug screening studies for emerging therapeutics targeted to MOR in pharmaceutical research, as well as the prolonged effects of such compounds on specifically the cell populations of interest they are targeted to with chronic treatment paradigms. Indeed, the broadening of opioid research models is further extended to in vitro culture systems, as evidenced in our use case for human-differentiated iPSCs. Induced pluripotent stem cells from pain patients are currently under development as powerful in vitro disease models that provide unique exploration of nociceptor mechanisms of pain, including use in high-throughput screens for novel analgesics and as diagnostics to identify individuals at risk for transitioning from acute to chronic pain. The great majority of peripheral nociceptors express MOR, and as demonstrated by the ability of the MORp constructs of the present disclosure to transduce human nociceptor iPSCs, additional embodiments can comprise the driving of expression of various gene editing tools or voltage indicators to be used in iPSCs for more complex phenotyping and screening studies.

The promise of gene therapy for addressing highly intractable neuropathologies at the level of the CNS and PNS may warrant further thought on the potential for viral constructs such as our own and others utilizing design strategies aimed at providing selective genetic access to specific cell populations, circuits or brain regions.

### Enumerated Embodiments

The following enumerated embodiments are provided, the numbering of which is not to be construed as designating levels of importance.

Embodiment 1 provides an isolated nucleic acid comprising a first region that encodes a Opoid Receptor promoter (ORP) region operably linked to a second region encoding an exogenous protein, wherein the opioid receptor promoter is selected from the group consisting of a mu opioid receptor promoter (MORP), a delta opioid receptor promoter (DORP), and a kappa opioid receptor promoter (KORP).

Embodiment 2 provides the isolated nucleic acid of embodiment 1, wherein the exogenous protein is a reporter protein.

Embodiment 3 provides the isolated nucleic acid of embodiment 2, wherein the reporter protein is selected from the group consisting of GFP, eYFP, mCherry, and GCaMP6f.

Embodiment 4 provides the isolated nucleic acid of embodiment 1, wherein the first and second regions are separated by a linker sequence.

Embodiment 5 provides the isolated nucleic acid of embodiment 4, wherein the linker sequence comprises a nucleic acid sequence selected set forth in SEQ ID NOs: 8 and 9.

Embodiment 6 provides the isolated nucleic acid of embodiment 1, wherein the MORP region is derived from a mouse MORP.

Embodiment 7 provides the isolated nucleic acid of embodiment 1, wherein the MORP regions is derived from a human MORP.

Embodiment 8 provides the isolated nucleic acid of embodiment 1, wherein the MORP region comprises a nucleic acid sequence selected from the group consisting of SEQ ID NOs: 6 and 7.

Embodiment 9 provides the isolated nucleic acid of embodiment 1, wherein the DORP region comprises a nucleic acid sequence selected from the group consisting of SEQ ID NOs: 13 and 14.

Embodiment 10 provides the isolated nucleic acid of embodiment 1, wherein the KORP region comprises a nucleic acid sequence set forth in SEQ ID NO: 15.

Embodiment 11 provides the isolated nucleic acid of embodiment 1, wherein the exogenous protein is a designer receptor exclusively activated by a designer drug (DREADD).

Embodiment 12 provides the isolated nucleic acid of embodiment 11, wherein the DREADD is encoded by a nucleic acid sequence comprising the nucleic acid sequence set forth in SEQ ID NO: 11.

Embodiment 13 provides the isolated nucleic acid of embodiment 11, wherein the designer drug agonist is selected from the group consisting of clozapine N-oxide (CNO) and deschloroclozapine (DCZ).

Embodiment 14 provides the isolated nucleic acid of embodiment 2, wherein the reporter protein is encoded by a nucleic acid sequence selected from the group consisting of SEQ ID NOs: 10 and 12.

Embodiment 15 provides the isolated nucleic acid of embodiment 1, further comprising a third region that encodes an additional exogenous protein.

Embodiment 16 provides the isolated nucleic acid of embodiment 15, wherein the third region is operably linked to the first region.

Embodiment 17 provides the isolated nucleic acid of embodiment 15, wherein the second region encodes a DREADD, and the third region encodes a reporter protein.

Embodiment 18 provides the isolated nucleic acid of embodiment 15, wherein the second region encodes a reporter protein, and the third region encodes a DREADD.

Embodiment 19 provides the isolated nucleic acid of embodiment 15, wherein the second region and third region are separated by a linker sequence.

Embodiment 20 provides the isolated nucleic acid of embodiment 19, wherein the linker sequence is a nucleic acid sequence selected from the group consisting of SEQ ID NOs: 8 and 9.

Embodiment 21 provides the isolated nucleic acid of embodiment 1, wherein the isolated nucleic acid comprises a nucleic acid sequence selected from the group consisting of SEQ ID NOs: 3, 4, and 5.

Embodiment 22 provides a recombinant Adeno-Associated Virus (AAV) vector comprising the isolated nucleic acid of any one of embodiments 1-21.

Embodiment 23 provides the recombinant AAV vector of embodiment 20, wherein the vector has a tropism for central nervous system (CNS) tissue.

Embodiment 24 provides the recombinant AAV vector of embodiment 20, wherein the vector comprises an AAV capsid protein of a serotype selected from the group consisting of AAV1, AAV2, AAV4, AAV5, AAV6, AAV7, AAV8, AAV9, AAV9P1, PHP.A, PHP.B, Rh8, Rh10, Rh43, Anc80L65, ShH10, ShH10Y, ShH19, Olig001, DJ, and TM6 Embodiment 25 provides the recombinant AAV vector of embodiment 22, wherein the vector comprises an AAV capsid protein of serotype AAV1 or a variant thereof.

Embodiment 26 provides the recombinant AAV vector of embodiment 22, wherein the vector comprises an AAV capsid protein of serotype AAV5 or a variant thereof.

Embodiment 27 provides a pharmaceutical composition comprising the recombinant AAV vector of any one of embodiments 22-26 and an acceptable carrier or excipient.

Embodiment 28 provides a method of assessing Opioid Receptor promoter ORP) activity in a cell, comprising contacting the cell with a vector comprising the isolated nucleic acid of any one of embodiments 1-21, wherein activity of the endogenous opioid receptor promoter drives activity of the ORP region of the isolated nucleic acid and expression of the exogenous protein encoded by the second or third regions, and wherein the opioid receptor promoter is selected from the group consisting of a mu opioid receptor promoter, a delta opioid receptor promoter, and a kappa opioid receptor promoter.

Embodiment 29 provides the method of embodiment 28, wherein the cell is a cell of the central nervous system (CNS).

Embodiment 30 provides the method of embodiment 28, wherein the cell is a neuron.

Embodiment 31 provides a method of providing analgesia to a subject in need thereof, comprising:
a. administering to the subject an effective amount of a composition comprising a recombinant AAV vector comprising an isolated nucleic acid comprising a first region encoding a ORP region operably linked to a second region encoding a designer receptor exclusively activated by a designer drug (DREADD),
wherein the DREADD is expressed by cells of the central nervous system; and
b. administering to the subject an effective amount of an agonist capable of engaging and activating the DREADD of part a., thereby providing analgesia,
wherein the ORP region is selected from the group consisting of a MORP region, a DORP region, and a KORP region.

Embodiment 32 provides the method of embodiment 31, wherein the composition further comprises a pharmaceutically acceptable carrier or excipient.

Embodiment 33 provides the method of embodiment 31, wherein the AAV vector comprises a capsid protein of a serotype selected from the group consisting of AAV1, AAV2, AAV4, AAV5, AAV6, AAV7, AAV8, AAV9, AAV9P1, PHP.A, PHP.B, Rh8, Rh10, Rh43, Anc80L65, ShH10, ShH10Y, ShH19, Olig001, DJ, and TM6.

Embodiment 34 provides the method of embodiment 31, wherein the AAV vector comprises a capsid protein of serotype AAV1 or a variant thereof.

Embodiment 35 provides the method of embodiment 31, wherein the AAV vector comprises a capsid protein of serotype AAV5 or a variant thereof.

Embodiment 36 provides the method of embodiment 31, wherein the DREADD is DREADD-Gi, also known as hM4Di.

Embodiment 37 provides the method of embodiment 36, wherein the DREADD-Gi is encoded by a nucleic acid sequence comprising the nucleic acid sequence set forth in SEQ ID NO: 11.

Embodiment 38 provides the method of embodiment 31, wherein the agonist is selected from the group consisting of clozapine-N-oxide (CNO) and deschloroclozapine (DCZ).

Embodiment 39 provides the method of embodiment 31, wherein the composition is administered before the administration of the ligand.

Embodiment 40 provides the method of embodiment 31, wherein the subject is a mammal.

Embodiment 41 provides the method of embodiment 31, wherein the subject is human.

### OTHER EMBODIMENTS

The recitation of a listing of elements in any definition of a variable herein includes definitions of that variable as any single element or combination (or subcombination) of listed elements. The recitation of an embodiment herein includes that embodiment as any single embodiment or in combination with any other embodiment or portions thereof.

The disclosures of each and every patent, patent application, and publication cited herein are hereby incorporated herein by reference in their entirety. While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

1. An isolated nucleic acid comprising a first region that encodes a Opioid Receptor promoter (ORP) region operably linked to a second region encoding an exogenous protein;
wherein the opioid receptor promoter is selected from the group consisting of a mu opioid receptor promoter (MORP), a delta opioid receptor promoter (DORP), and a kappa opioid receptor promoter (KORP).

2. The isolated nucleic acid of claim 1, wherein the exogenous protein is a reporter protein.

3. The isolated nucleic acid of claim 2, wherein the reporter protein is selected from the group consisting of GFP, eYFP, mCherry, and GCaMP6f.

4. The isolated nucleic acid of claim 1, wherein the first and second regions are separated by a linker sequence.

5. The isolated nucleic acid of claim 4, wherein the linker sequence comprises a nucleic acid sequence selected set forth in SEQ ID NOs: 8 and 9.

6. The isolated nucleic acid of claim 1, wherein the MORP region is derived from a mouse MORP.

7. The isolated nucleic acid of claim 1, wherein the MORP regions is derived from a human MORP.

8. The isolated nucleic acid of claim 1, wherein the MORP region comprises a nucleic acid sequence selected from the group consisting of SEQ ID NOs: 6 and 7.

9. The isolated nucleic acid of claim 1, wherein the DORP region comprises a nucleic acid sequence selected from the group consisting of SEQ ID NOs: 13 and 14.

10. The isolated nucleic acid of claim 1, wherein the KORP region comprises a nucleic acid sequence set forth in SEQ ID NO: 15.

11. The isolated nucleic acid of claim 1, wherein the exogenous protein is a designer receptor exclusively activated by a designer drug (DREADD).

12. The isolated nucleic acid of claim 11, wherein the DREADD is encoded by a nucleic acid sequence comprising the nucleic acid sequence set forth in SEQ ID NO: 11.

13. The isolated nucleic acid of claim 11, wherein the designer drug agonist is selected from the group consisting of clozapine N-oxide (CNO) and deschloroclozapine (DCZ).

14. The isolated nucleic acid of claim 2, wherein the reporter protein is encoded by a nucleic acid sequence selected from the group consisting of SEQ ID NOs: 10 and 12.

15. The isolated nucleic acid of claim 1, further comprising a third region that encodes an additional exogenous protein.

16. The isolated nucleic acid of claim 15, wherein the third region is operably linked to the first region.

17. The isolated nucleic acid of claim 15, wherein the second region encodes a DREADD, and the third region encodes a reporter protein.

18. The isolated nucleic acid of claim 15, wherein the second region encodes a reporter protein, and the third region encodes a DREADD.

19. The isolated nucleic acid of claim 15, wherein the second region and third region are separated by a linker sequence.

20. The isolated nucleic acid of claim 19, wherein the linker sequence is a nucleic acid sequence selected from the group consisting of SEQ ID NOs: 8 and 9.

21. The isolated nucleic acid of claim 1, wherein the isolated nucleic acid comprises a nucleic acid sequence selected from the group consisting of SEQ ID NOs: 3, 4, and 5.

22. A recombinant Adeno-Associated Virus (AAV) vector comprising the isolated nucleic acid of claim 1.

23. The recombinant AAV vector of claim 22, wherein the vector has a tropism for central nervous system (CNS) tissue.

24. The recombinant AAV vector of claim 22, wherein the vector comprises an AAV capsid protein of a serotype selected from the group consisting of AAV1, AAV2, AAV4, AAV5, AAV6, AAV7, AAV8, AAV9, AAV9P1, PUPA, PHP.B, Rh8, Rh10, Rh43, Anc80L65, ShH10, ShH10Y, ShH19, Olig001, DJ, and TM6.

25. The recombinant AAV vector of claim 22, wherein the vector comprises an AAV capsid protein of serotype AAV1 or a variant thereof.

26. The recombinant AAV vector of claim 22, wherein the vector comprises an AAV capsid protein of serotype AAV5 or a variant thereof.

27. A pharmaceutical composition comprising the recombinant AAV vector of claim 22 and an acceptable carrier or excipient.

28. A method of assessing Opioid Receptor promoter (ORP) activity in a cell, comprising contacting the cell with a vector comprising the isolated nucleic acid of claim 1,
wherein activity of the endogenous opioid receptor promoter drives activity of the ORP region of the isolated nucleic acid and expression of the exogenous protein encoded by the second or third regions, and
wherein the opioid receptor promoter is selected from the group consisting of a mu opioid receptor promoter, a delta opioid receptor promoter, and a kappa opioid receptor promoter.

29. The method of claim 28, wherein the cell is a cell of the central nervous system (CNS).

30. The method of claim 28, wherein the cell is a neuron.

31. A method of providing analgesia to a subject in need thereof, comprising:
a. administering to the subject an effective amount of a composition comprising a recombinant AAV vector comprising an isolated nucleic acid comprising a first region encoding a ORP region operably linked to a second region encoding a designer receptor exclusively activated by a designer drug (DREADD),
wherein the DREADD is expressed by cells of the central nervous system; and
b. administering to the subject an effective amount of an agonist capable of engaging and activating the DREADD of part a., thereby providing analgesia,
wherein the ORP region is selected from the group consisting of a MORP region, a DORP region, and a KORP region.

32. The method of claim 31, wherein the composition further comprises a pharmaceutically acceptable carrier or excipient.

33. The method of claim 31, wherein the AAV vector comprises a capsid protein of a serotype selected from the group consisting of AAV1, AAV2, AAV4, AAV5, AAV6, AAV7, AAV8, AAV9, AAV9P1, PHP.A, PHP.B, Rh8, Rh10, Rh43, Anc80L65, ShH10, ShH10Y, ShH19, Olig001, DJ, and TM6.

34. The method of claim 31, wherein the AAV vector comprises a capsid protein of serotype AAV1 or a variant thereof.

35. The method of claim 31, wherein the AAV vector comprises a capsid protein of serotype AAV5 or a variant thereof.

36. The method of claim 31, wherein the DREADD is DREADD-Gi, also known as hM4Di.

37. The method of claim 36, wherein the DREADD-Gi is encoded by a nucleic acid sequence comprising the nucleic acid sequence set forth in SEQ ID NO: 11.

38. The method of claim 31, wherein the agonist is selected from the group consisting of clozapine-N-oxide (CNO) and deschloroclozapine (DCZ).

39. The method of claim 31, wherein the composition is administered before the administration of the ligand.

40. The method of claim 31, wherein the subject is a mammal.

41. The method of claim 31, wherein the subject is human.


SEQUENCE LISTING (15 sequences)

SEQ ID NO: 1 - DNA, 34 bp, synthetic construct
  source 1..34: mol_type = other DNA
cgcacgcgtgagaacatatggttggacaaaattc

SEQ ID NO: 2 - DNA, 35 bp, synthetic construct
  source 1..35: mol_type = other DNA
ggcaccggtggaagggagggagcatgggctgtgag

SEQ ID NO: 3 - DNA, 2264 bp, synthetic construct
  source 1..2264: mol_type = other DNA
gagaacatatggttggacaaaattcaggtttgtactctgagaagaagactgaggtaaagtagcatcacactgcgagagattacagggctagatcacaaaatgatctgatagcaaaggcagcagtaagcaccacaagtcaacctgtcattcacaagaagggggtaaagatggttctgtgtgtcctggggagaccgatgaatgtgagcacacagatttcagctcagccatatgaactgacacacttattccgaagagaatatatttaaaggtattttagcagcctctagaaaaccttatctacattactgctgaaatgacaccctttaacgcaaaaggggacaaataatactgaacgttgtccaaaagtctacaatagttgagctgtgtgttatctttttttcagaatgattgcttaaaagtaaaaatatatgttccttctaagcttgcttccccacagtctgtgagaattcagttaaacttctacaaccaaatgggagcggggacatttatttccagtatttatagttaaaactatgggtatgcgcatggcatgcatatgcatacacagtgttgagacgaaaccacactactgctgaatttacgattgtattccttcagaagcacttgtgaacttagacatgagatctaacattttttaaatgcattcttgctcctcaaggatcttgcttaaggccatttcacactcaaaagagctgtggagtttcttttggattctctatagtgggaagaaaaaacatttctgcttgattcagtaacctgctccccagaagctcgggagagtggaggcagtgcgaaagcttaggataaatcttcctaagcaggtccaggagagtagacctggcatgggtagtgggttccttatttacaggttatcacccctcctccctcacatctctaatgtgttcttatgcttcttcttcactaggaaaaaaatcagatgcaaatagaccttctaacctaagagaaattgaaggagtgggggcacatgaaacaggcttctttcacaaaaattttctcttttagaaaaagacaattgtttctttgaacaggtttgtgggggttgattttttgaggaactaaaggggccttttgttttgttttgttttggtttgtttgtttgttttccctcagatatggggatgtgggtaaaggacaacattaataaccctccagtcctaattaattgcatatggttagttgagtcggaagagtgttgaggtataagggtcattgagattttaacagaccaacgactttcctggctgatgcaaacaacctcaaacaacctaatgcatgttgttacaaagaaacttagagtctctcttcccaccctctctgcagcatcccgcttctgctccccccccccctaccccaatttacactcctttacacggaagtgcatagagactgaggaggctgattctgagttgctttgcgcacaatccactccttctctctcctccctcccctctagcctctggatccctcacagcccatgctccctcccttccaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaa

SEQ ID NO: 4 - DNA, 3701 bp, synthetic construct
  source 1..3701: mol_type = other DNA
gagaacatatggttggacaaaattcaggtttgtactctgagaagaagactgaggtaaagtagcatcacactgcgagagattacagggctagatcacaaaatgatctgatagcaaaggcagcagtaagcaccacaagtcaacctgtcattcacaagaagggggtaaagatggttctgtgtgtcctggggagaccgatgaatgtgagcacacagatttcagctcagccatatgaactgacacacttattccgaagagaatatatttaaaggtattttagcagcctctagaaaaccttatctacattactgctgaaatgacaccctttaacgcaaaaggggacaaataatactgaacgttgtccaaaagtctacaatagttgagctgtgtgttatctttttttcagaatgattgcttaaaagtaaaaatatatgttccttctaagcttgcttccccacagtctgtgagaattcagttaaacttctacaaccaaatgggagcggggacatttatttccagtatttatagttaaaactatgggtatgcgcatggcatgcatatgcatacacagtgttgagacgaaaccacactactgctgaatttacgattgtattccttcagaagcacttgtgaacttagacatgagatctaacattttttaaatgcattcttgctcctcaaggatcttgcttaaggccatttcacactcaaaagagctgtggagtttcttttggattctctatagtgggaagaaaaaacatttctgcttgattcagtaacctgctccccagaagctcgggagagtggaggcagtgcgaaagcttaggataaatcttcctaagcaggtccaggagagtagacctggcatgggtagtgggttccttatttacaggttatcacccctcctccctcacatctctaatgtgttcttatgcttcttcttcactaggaaaaaaatcagatgcaaatagaccttctaacctaagagaaattgaaggagtgggggcacatgaaacaggcttctttcacaaaaattttctcttttagaaaaagacaattgtttctttgaacaggtttgtgggggttgattttttgaggaactaaaggggccttttgttttgttttgttttggtttgtttgtttgttttccctcagatatggggatgtgggtaaaggacaacattaataaccctccagtcctaattaattgcatatggttagttgagtcggaagagtgttgaggtataagggtcattgagattttaacagaccaacgactttcctggctgatgcaaacaacctcaaacaacctaatgcatgttgttacaaagaaacttagagtctctcttcccaccctctctgcagcatcccgcttctgctccccccccccctaccccaatttacactcctttacacggaagtgcatagagactgaggaggctgattctgagttgctttgcgcacaatccactccttctctctcctccctcccctctagcctctggatccctcacagcccatgctccctcccttccaccggtgccaccatggccaacttcacacctgtcaatggcagctcgggcaatcagtccgtgcgcctggtcacgtcatcatcccacaatcgctatgagacggtggaaatggtcttcattgccacagtgacaggctccctgagcctggtgactgtcgtgggcaacatcctggtgatgctgtccatcaaggtcaacaggcagctgcagacagtcaacaactacttcctcttcagcctggcgtgtgctgatctcatcataggcgccttctccatgaacctctacaccgtgtacatcatcaagggctactggcccctgggcgccgtggtctgcgacctgtggctggccctggactgcgtggtgagcaacgcctccgtcatgaaccttctcatcatcagctttgaccgctacttctgcgtcaccaagcctctcacctaccctgcccggcgcaccaccaagatggcaggcctcatgattgctgctgcctgggtactgtccttcgtgctctgggcgcctgccatcttgttctggcagtttgtggtgggtaagcggacggtgcccgacaaccagtgcttcatccagttcctgtccaacccagcagtgacctttggcacagccattgctggcttctacctgcctgtggtcatcatgacggtgctgtacatccacatctccctggccagtcgcagccgagtccacaagcaccggcccgagggcccgaaggagaagaaagccaagacgctggccttcctcaagagcccactaatgaagcagagcgtcaagaagcccccgcccggggaggccgcccgggaggagctgcgcaatggcaagctggaggaggcccccccgccagcgctgccaccgccaccgcgccccgtggctgataaggacacttccaatgagtccagctcaggcagtgccacccagaacaccaaggaacgcccagccacagagctgtccaccacagaggccaccacgcccgccatgcccgcccctcccctgcagccgcgggccctcaacccagcctccagatggtccaagatccagattgtgacgaagcagacaggcaatgagtgtgtgacagccattgagattgtgcctgccacgccggctggcatgcgccctgcggccaacgtggcccgcaagttcgccagcatcgctcgcaaccaggtgcgcaagaagcggcagatggcggcccgggagcgcaaagtgacacgaacgatctttgccattctgctggccttcatcctcacctggacgccctacaacgtcatggtcctggtgaacaccttctgccagagctgcatccctgacacggtgtggtccattggctactggctctgctacgtcaacagcaccatcaaccctgcctgctatgctctgtgcaacgccacctttaaaaagaccttccggcacctgctgctgtgccagtatcggaacatcggcactgccaggcgggcggccgccgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 5 - DNA, 2316 bp, synthetic construct
  source 1..2316: mol_type = other DNA
cctgctaacagacatatgaggtgaatattattagcctaccctcgccttttttaagtaatgagaagactgtcatcctgtagggtaaagtaacatgtccaaactcatacagctacaaagttacaaagctgatttataaaatgatttgactccaaggtcaggaattattatactgtgtcttgtcttccacatgaactaagcacaaaggaactgaatgcaggcagacagatttcagctcaatataagagaattgttacattagttcatggaagaatatgttttaaggtatttttgttagtctctaggaaatctctgtaacattttattgtgtaaattatatgctttaatgtaagaggataaaaataatagtgaacattggcaaaatagcctatgattaatagagtttacctatgagttatctgtttctaagataaatgccaaaaaataatattggaattaaatgttcctttcaagatcttccctccctgctccctgaaattgcagtgaatttttcaagaccaactgaggacatgtattttcaatgtttatggttaaaagatatgtacatgcacagatatatacatgtacagaaatgagaattacttcagaattggtgttaactttagaaaaaaaaagaccaagaacttactcttggtatttacaaatttatttctaaaatagaagcactcatggacttagaagtaaggtataaaattcaaaaacgtatccatgtttctcaaggatcttgttgtaggccactctaattccatatattatgtggcttttcctagaatttttacactagaaaacagactgaatgcaaattttgttttgttttaacaaccttcttctcagaagcatatgtctatcgaggaagtcttcagataaaaaagataaacaattccaaacaggtctatgagatttaagatgtgaaagatcaacattatctttagttgactttactggatgccacaaccttctgatttctgtaaccacttcttatgcctcctacccactgaaacaaaatcagaggcaaacagagcttcaccctagaaattggggaaaatgaggaacaggttttctgcacaaaagtttatttgtttctcatttctttttcagaaaataaaggatcgctgttgttcccaacaggtttgtagggaagaaaattggagaaacattattaccttttcttagatgttggcaacggaggcaacaaggactgcaaaagaaaattgtgtgtcccccattcctaaataatcaaaattggcagtagggatggaagagcattggggttttagggctgttagggtttcatcaagccaatgtattccctgccagattttaaggagaaaaaggcgctggaaaattgagtgatgttagccccctttcttatttttcactgctaccaaagactaactctatctctctccccaacccttctctccatctccctcctttagatgtgtttgcacagaagagtgcccagtgaagagacctactccttggatcgctttgcgcaaaatccaccccttttccctcctccctcccttccagcctccgaatcccgcatggcccacgctcccctccaccggtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaa

SEQ ID NO: 6 - DNA, 1532 bp, synthetic construct
  source 1..1532: mol_type = other DNA
gagaacatatggttggacaaaattcaggtttgtactctgagaagaagactgaggtaaagtagcatcacactgcgagagattacagggctagatcacaaaatgatctgatagcaaaggcagcagtaagcaccacaagtcaacctgtcattcacaagaagggggtaaagatggttctgtgtgtcctggggagaccgatgaatgtgagcacacagatttcagctcagccatatgaactgacacacttattccgaagagaatatatttaaaggtattttagcagcctctagaaaaccttatctacattactgctgaaatgacaccctttaacgcaaaaggggacaaataatactgaacgttgtccaaaagtctacaatagttgagctgtgtgttatctttttttcagaatgattgcttaaaagtaaaaatatatgttccttctaagcttgcttccccacagtctgtgagaattcagttaaacttctacaaccaaatgggagcggggacatttatttccagtatttatagttaaaactatgggtatgcgcatggcatgcatatgcatacacagtgttgagacgaaaccacactactgctgaatttacgattgtattccttcagaagcacttgtgaacttagacatgagatctaacattttttaaatgcattcttgctcctcaaggatcttgcttaaggccatttcacactcaaaagagctgtggagtttcttttggattctctatagtgggaagaaaaaacatttctgcttgattcagtaacctgctccccagaagctcgggagagtggaggcagtgcgaaagcttaggataaatcttcctaagcaggtccaggagagtagacctggcatgggtagtgggttccttatttacaggttatcacccctcctccctcacatctctaatgtgttcttatgcttcttcttcactaggaaaaaaatcagatgcaaatagaccttctaacctaagagaaattgaaggagtgggggcacatgaaacaggcttctttcacaaaaattttctcttttagaaaaagacaattgtttctttgaacaggtttgtgggggttgattttttgaggaactaaaggggccttttgttttgttttgttttggtttgtttgtttgttttccctcagatatggggatgtgggtaaaggacaacattaataaccctccagtcctaattaattgcatatggttagttgagtcggaagagtgttgaggtataagggtcattgagattttaacagaccaacgactttcctggctgatgcaaacaacctcaaacaacctaatgcatgttgttacaaagaaacttagagtctctcttcccaccctctctgcagcatcccgcttctgctccccccccccctaccccaatttacactcctttacacggaagtgcatagagactgaggaggctgattctgagttgctttgcgcacaatccactccttctctctcctccctcccctctagcctctggatccctcacagcccatgctccctcccttcc

SEQ ID NO: 7 - DNA, 1584 bp, synthetic construct
  source 1..1584: mol_type = other DNA
cctgctaacagacatatgaggtgaatattattagcctaccctcgccttttttaagtaatgagaagactgtcatcctgtagggtaaagtaacatgtccaaactcatacagctacaaagttacaaagctgatttataaaatgatttgactccaaggtcaggaattattatactgtgtcttgtcttccacatgaactaagcacaaaggaactgaatgcaggcagacagatttcagctcaatataagagaattgttacattagttcatggaagaatatgttttaaggtatttttgttagtctctaggaaatctctgtaacattttattgtgtaaattatatgctttaatgtaagaggataaaaataatagtgaacattggcaaaatagcctatgattaatagagtttacctatgagttatctgtttctaagataaatgccaaaaaataatattggaattaaatgttcctttcaagatcttccctccctgctccctgaaattgcagtgaatttttcaagaccaactgaggacatgtattttcaatgtttatggttaaaagatatgtacatgcacagatatatacatgtacagaaatgagaattacttcagaattggtgttaactttagaaaaaaaaagaccaagaacttactcttggtatttacaaatttatttctaaaatagaagcactcatggacttagaagtaaggtataaaattcaaaaacgtatccatgtttctcaaggatcttgttgtaggccactctaattccatatattatgtggcttttcctagaatttttacactagaaaacagactgaatgcaaattttgttttgttttaacaaccttcttctcagaagcatatgtctatcgaggaagtcttcagataaaaaagataaacaattccaaacaggtctatgagatttaagatgtgaaagatcaacattatctttagttgactttactggatgccacaaccttctgatttctgtaaccacttcttatgcctcctacccactgaaacaaaatcagaggcaaacagagcttcaccctagaaattggggaaaatgaggaacaggttttctgcacaaaagtttatttgtttctcatttctttttcagaaaataaaggatcgctgttgttcccaacaggtttgtagggaagaaaattggagaaacattattaccttttcttagatgttggcaacggaggcaacaaggactgcaaaagaaaattgtgtgtcccccattcctaaataatcaaaattggcagtagggatggaagagcattggggttttagggctgttagggtttcatcaagccaatgtattccctgccagattttaaggagaaaaaggcgctggaaaattgagtgatgttagccccctttcttatttttcactgctaccaaagactaactctatctctctccccaacccttctctccatctccctcctttagatgtgtttgcacagaagagtgcccagtgaagagacctactccttggatcgctttgcgcaaaatccaccccttttccctcctccctcccttccagcctccgaatcccgcatggcccacgctcccctcc

SEQ ID NO: 8 - DNA, 12 bp, synthetic construct
  source 1..12: mol_type = other DNA
accggtgccacc

SEQ ID NO: 9 - skipped

SEQ ID NO: 10 - DNA, 720 bp, synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaa

SEQ ID NO: 11 - DNA, 1440 bp, synthetic construct
  source 1..1440: mol_type = other DNA
atggccaacttcacacctgtcaatggcagctcgggcaatcagtccgtgcgcctggtcacgtcatcatcccacaatcgctatgagacggtggaaatggtcttcattgccacagtgacaggctccctgagcctggtgactgtcgtgggcaacatcctggtgatgctgtccatcaaggtcaacaggcagctgcagacagtcaacaactacttcctcttcagcctggcgtgtgctgatctcatcataggcgccttctccatgaacctctacaccgtgtacatcatcaagggctactggcccctgggcgccgtggtctgcgacctgtggctggccctggactgcgtggtgagcaacgcctccgtcatgaaccttctcatcatcagctttgaccgctacttctgcgtcaccaagcctctcacctaccctgcccggcgcaccaccaagatggcaggcctcatgattgctgctgcctgggtactgtccttcgtgctctgggcgcctgccatcttgttctggcagtttgtggtgggtaagcggacggtgcccgacaaccagtgcttcatccagttcctgtccaacccagcagtgacctttggcacagccattgctggcttctacctgcctgtggtcatcatgacggtgctgtacatccacatctccctggccagtcgcagccgagtccacaagcaccggcccgagggcccgaaggagaagaaagccaagacgctggccttcctcaagagcccactaatgaagcagagcgtcaagaagcccccgcccggggaggccgcccgggaggagctgcgcaatggcaagctggaggaggcccccccgccagcgctgccaccgccaccgcgccccgtggctgataaggacacttccaatgagtccagctcaggcagtgccacccagaacaccaaggaacgcccagccacagagctgtccaccacagaggccaccacgcccgccatgcccgcccctcccctgcagccgcgggccctcaacccagcctccagatggtccaagatccagattgtgacgaagcagacaggcaatgagtgtgtgacagccattgagattgtgcctgccacgccggctggcatgcgccctgcggccaacgtggcccgcaagttcgccagcatcgctcgcaaccaggtgcgcaagaagcggcagatggcggcccgggagcgcaaagtgacacgaacgatctttgccattctgctggccttcatcctcacctggacgccctacaacgtcatggtcctggtgaacaccttctgccagagctgcatccctgacacggtgtggtccattggctactggctctgctacgtcaacagcaccatcaaccctgcctgctatgctctgtgcaacgccacctttaaaaagaccttccggcacctgctgctgtgccagtatcggaacatcggcactgccaggcgg

SEQ ID NO: 12 - DNA, 708 bp, synthetic construct
  source 1..708: mol_type = other DNA
gtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 13 - DNA, 1673 bp, synthetic construct
  source 1..1673: mol_type = other DNA
gggtcttgctgtcatccaggctggagtgcaggggcaagatctcgactcactgcagcctctattttcccagctcaaatgatcctcccacctcagcctcccaagtagctgcaactagagacacacattaccattcctggctaattttttttttttttttttttttttttttgtagagacagggttttgccatgttgcccacgctggtttcgaactcctaggctcaagtgatctgtctgcctcagcctctcaaaatgttcggattacaagtgtgtgccaccgtgcccagcctttttcaatttttcttgtagatccaagggatccatgtgcagatttgccacctgggtatattttgtgatgctgaggtttggggtatgaattatcctgtcaactaagttactgagcatagtattcaatggttagttttacacaatacttttttgtgtgaaggtgatgcacaaaaaatccaatgattacacaataaatacatgggaaactattaatatgtaaagaagaagtacgtgatgctactgcataataaggagagctgggctagttggaggtggggtggggatcagggaaggtttcacaaaggaagtgatatttaagctgaaatttgaggggtaaataacgcttaggctggggtattggcccagccttccgagtggaaggaatagcatgtacaaatgtcctgaggtaggaaaaaacttggcttggagggttagggaggggaccagaattccaataaagtagccctccttcttcctatcccaatttctctctgctggcactagaaaagtttctgagtaccaggccctttgggaaagggagagagttttggcagcaggtgaccacagcaaaaatctttctgataagtccttaggtggttgagtgggaaagccctggggtggggggcttgctgtagttggtagaagtggctggacagcagctaagtggggtggagaaaatggagaagagggtgagcttgggaattctggctttgcttttttctgggaaagggtaggagccttgcacatatatggatgattcaagctagagggtgagatgagggagagctccttgtccagccacacacagatccaagagcattgccccagagcctagagctcagaacccctaatattgttccactccagttccctttgggatctttattatccaaaaagtatttcttgagcacctgctgtgaccccagccctctcctggatgcaggggtcaatcagacctggtccctttaaggagggagaccagttcggatagaaataattactatatgggataatttagtgctgagcactaaatctgtaacatggggacaataataatagaagctacaccaaagtgttggtgcatggataaagtgcttatcacagtgcctggagcaaagtaatgcttggtagaatgttctggaaaggctaggcaccccaagaagaaggatggctctaatctggaatggttaggtcttggaaggggtgtggcaaggatgacttagccaagtgccccccttctatctgctcgcactgcagccagagtgctccttaaggtggtcttcaagagcctgcccaccacctcatcgccccctcacttcctgccttctcctcttcctggcttcctttcagtccctctgcttcctttatccggaatcctcttccctt

SEQ ID NO: 14 - DNA, 1501 bp, synthetic construct
  source 1..1501: mol_type = other DNA
gagacagggttttgccatgttgcccacgctggtttcgaactcctaggctcaagtgatctgtctgcctcagcctctcaaaatgttcggattacaagtgtgtgccaccgtgcccagcctttttcaatttttcttgtagatccaagggatccatgtgcagatttgccacctgggtatattttgtgatgctgaggtttggggtatgaattatcctgtcaactaagttactgagcatagtattcaatggttagttttacacaatacttttttgtgtgaaggtgatgcacaaaaaatccaatgattacacaataaatacatgggaaactattaatatgtaaagaagaagtacgtgatgctactgcataataaggagagctgggctagttggaggtggggtggggatcagggaaggtttcacaaaggaagtgatatttaagctgaaatttgaggggtaaataacgcttaggctggggtattggcccagccttccgagtggaaggaatagcatgtacaaatgtcctgaggtaggaaaaaacttggcttggagggttagggaggggaccagaattccaataaagtagccctccttcttcctatcccaatttctctctgctggcactagaaaagtttctgagtaccaggccctttgggaaagggagagagttttggcagcaggtgaccacagcaaaaatctttctgataagtccttaggtggttgagtgggaaagccctggggtggggggcttgctgtagttggtagaagtggctggacagcagctaagtggggtggagaaaatggagaagagggtgagcttgggaattctggctttgcttttttctgggaaagggtaggagccttgcacatatatggatgattcaagctagagggtgagatgagggagagctccttgtccagccacacacagatccaagagcattgccccagagcctagagctcagaacccctaatattgttccactccagttccctttgggatctttattatccaaaaagtatttcttgagcacctgctgtgaccccagccctctcctggatgcaggggtcaatcagacctggtccctttaaggagggagaccagttcggatagaaataattactatatgggataatttagtgctgagcactaaatctgtaacatggggacaataataatagaagctacaccaaagtgttggtgcatggataaagtgcttatcacagtgcctggagcaaagtaatgcttggtagaatgttctggaaaggctaggcaccccaagaagaaggatggctctaatctggaatggttaggtcttggaaggggtgtggcaaggatgacttagccaagtgccccccttctatctgctcgcactgcagccagagtgctccttaaggtggtcttcaagagcctgcccaccacctcatcgccccctcacttcctgccttctcctcttcctggcttcctttcagtccctctgcttcctttatccggaatcctcttccctt

SEQ ID NO: 15 - DNA, 2120 bp, synthetic construct
  source 1..2120: mol_type = other DNA
gaggattgcttgaagatctatatgttgaatatgtgtcctcaaacctttaatagttgcaaagtagttggaagtctttggataattttaagtgtggccttaaatgtattcttgggacttcatgcccctcttcctattcttcattactggtgtcaaaacgaatggctttgctctgctgtgctcttctgtcttgagatgctgctttgtcataggcctgaaacaacaaagccagcaaggcatgctctagaacctccaagaaagaaagctcaagtatgtgttttcatgaggcaaaactgactagtacaccctaaaaaagtttatctgttcaatggatggataaaaataaaagaccaggacccaggtatcactaattttataaagcaaagaatgccagtataaggtcatgcagacacaggaaccttagtaactcaaaacacaagagtagatgccaaagctattacttgctggaatctattagagattctgttagaggaagttgaagcagtggatattatatctataacctaaactttttaaacatgaaattgtattttaaaacaggcagtattcatgtatattgcattctgtattttttcattagaaatattacacgattgtggttgcttagttatctgtgtgtttagaacgatattatgagtcttttgttatagagttttaatcactttctaacacatataaccacttaacaatatttccataatatgaccaaggccaggaagtatttccccactccccagagtaaatcactctttctgaaatggatcataaaggtgcaaatcagaacatgctgtctttagtactaatccttgcagtgcccacctacctagtcaataacatctctcctttctcacagaacaaggatagatcgggaagacaacgctgctgagggtggaatgcattcctgtctgacagatgtatcaagctagagcctagactggcctctcttctaagctcccagcttagtatccttacacatcaggaacggactcaggaaatttagactcagaactgtgacttcacattagacaacaccatttcatcactctgatgtcactgacactgacagctaacatcttaaagagaaaatgtatatatatatttatacatgtatatatatatatatagacagaaattaatttattgtgatgaggttcataattctgaggtacagagaaaggaattaacacacagctttttcctcttggcttctgagaggtaagacactacgggtatttactggtcaatgacttctggaagaagcatttccttcagagaatgattctgctgattcagatttgaatgaaaatgctgtcactctaaacttactgctgtagtatagagaatgatttcagtttccaaacaatattgctgacctatcgtgaacattttgttcccttgtctgaatagtaagacaatggtgcactataaaaaaactttccagatgtgggatggagaaaaccagttttatgtactgtggttttgttgtatcaaatataattcatcttgatctgttttacttatataatgtactatgatgataagttagtagcatactgccatgcagaaaccatagagtctaattgtcttttggccactgcagatgggtatattttaaaattgaacaccattttgtactttataaaggttaaataattacaattattgatttcttaaaaataaacattaaggtatttttttatacagaaaagaaagatacttcaagaatacttaagtcaagaccactgtgaaggattaaaatgaatcatcacctctgacatgaaggactccatagtattccagcctgtttgcatattcatctagctattaatctatgctgtattccttctcatttttatttaacttatttaatcaaagcactgagatagaaccttaatgatgcctgaagtctttccttatgtttctcaataccctggtatctaggaagtccttttaaaatctacacacacaatgagtgcataagaaataggtacctgcaaagcaaaattaattattaagtggctgtcactctcattcacactctgggtttaagttgcagacatggaaggaaatgttactgtttcttattttttagatacacgaag